(19) United States
(12) Patent Application Publication
(10) Pub. No.: US 2005/0164929 A1
(43) Pub. Date: Jul. 28, 2005

(54) Methods of preventing and treating inflammatory bowel disease

(75) Inventors: Enrique Alvarez, Clinton, CT (US); Jeffrey Peterson, Guilford, CT (US); William J. LaRochelle, Madison, CT (US); Henri Lichenstein, Guilford, CT (US); Michael E. Jeffers, Branford, CT (US)

Correspondence Address: Jenell Lawson;Intellectual Property, CuraGen Corporation, 555 Long Wharf Drive, New Haven, CT, 06551, US

(73) Assignee: LUPINE LOGIC, INC., Missoula, MT (US)

(21) Appl. No.: 10/980,764

(22) Filed: Nov. 3, 2004

Related U.S. Application Data
(63) Continuation-in-part of application No. 10/321,962, filed on Dec. 16, 2002.
(63) Continuation-in-part of application No. 10/011,364, filed on Nov. 16, 2001.
Application No. 10/011,364 is a continuation-in-part of application No. 09/992,840, filed on Nov. 6, 2001.
(60) Provisional application No. 60/386,545, filed on Jun. 6, 2002.
(60) Provisional application No. 60/246,206, filed on Nov. 6, 2000.

(51) Int. Cl. A61K 48/00; A61K 38/17

(52) U.S. Cl. 514/12; 514/44

(57) ABSTRACT

The present invention relates to compositions and methods for preventing and treating an inflammatory disorder of a mucosa, including but is not limited to, inflammatory bowel disease and irritable bowel syndrome. More particularly, the present invention relates to compositions comprising FGF-20, a fragment, a derivative, a variant, a homolog, or an analog thereof, and their uses in preventing and treating an inflammatory disorder of a mucosa, such as inflammatory bowel disease or irritable bowel syndrome.

This application is a continuation-in-part of the U.S. patent application Ser. No. 10/321,962, filed Dec. 16, 2002, which claims the priority benefit to U.S. Provisional Application No. 60/386,545, filed Jun. 6, 2002, and is a continuation-in-part of the U.S. patent application Ser. No. 10/011,364, filed Nov. 16, 2001, which in turn is a continuation-in-part of the U.S. patent application Ser. No. 09/992,840, filed Nov. 6, 2001, which claims the priority benefit to the U.S. Provisional Application No. 60/246,206, filed Nov. 6, 2000. The content of each application is incorporated herein by reference in its entirety.

### 1. FIELD OF THE INVENTION

The present invention relates to compositions and methods for preventing and treating an inflammatory disorder of a mucosa, including but is not limited to, inflammatory bowel disease and irritable bowel syndrome. More particularly, the present invention relates to compositions comprising FGF-20, a fragment, a derivative, a variant, a homolog, or an analog thereof, and their uses in preventing and treating an inflammatory disorder of a mucosa, such as inflammatory bowel disease or irritable bowel syndrome.

### 2. BACKGROUND OF THE INVENTION

2.1 Inflammatory Bowel Disease

Inflammatory bowel disease (“IBD”) refers to a group of chronic inflammatory disorders involving the gastrointestinal tract. Although IBD is diagnosed largely by exclusion, there are characteristic features associated with it that allows accurate diagnosis.

Chronic IBD is sub-divided into two major groups, namely, ulcerative colitis (“UC”) and Crohn's disease (“CD”). Clinically, IBD is characterized by recurrent inflammatory involvement of intestinal segments with diverse clinical manifestations. Typically UC affects the rectum and extends proximally to involve part or all of the colon. Lesions are restricted to the mucosal or submucosal layers of the colon with deeper layers unaffected except in fulminant disease (see, e.g., Friedman S, Blumberg RS. Inflammatory bowel disease. In: Braunwald E, Fauci A S, Kasper D L, Hauser S L, Longo D L, Jameson J L, editors. Harrison's Principles of Internal Medicine. 15th ed. New York: McGraw-Hill; 2001. pp 1679-692). Symptoms include, e.g., rectal bleeding, mucus containing diarrhea, abdominal pain and weight loss. Clinical manifestation alternates with quiescent remission (see, e.g., Miner P B. Clinical features, course, laboratory findings, and complications in ulcerative colitis. In: Kirsner J B, editor. Inflammatory Bowel Disease. 5th ed. Philadelphia (PA): W.B. Saunders Company; 1999. pp 299-304).

In comparison, CD generally affects the full thickness of the gut wall in both the small and large intestines. The clinical symptoms of UC vary according to the region affected. In general, fever, malaise, weight loss, abdominal pain and cramps are the common symptoms of CD. Full thickness bowel lesions can progress to bowel perforations and local abscesses, fistulas in the adjoining abdominal and pelvic organs, and fibrosis of the bowel wall with obstruction. In small bowel disease, significant malabsorption of many nutrients including Vitamins B12 and D, bile salts, protein, calcium, and magnesium may result (see, e.g., Lashner B A. Clinical features, laboratory findings, and course of Crohn's disease. In: Kirsner J B, editor. Inflammatory Bowel Disease. 5th ed. Philadelphia (PA): W.B. Saunders Company; 1999. pp 305-314).

The etiology of UC or CD remains unknown. However, a combination of factors, including but are not limited to, abnormalities in the immune system, genetic predisposition, environmental and psychological factors, may be of importance in determining the outcome of the disease.

In Europe and the United States, incidence and prevalence of CD is approximately 1-6 and 10-100 cases per 100,000 population respectively. For UC, the incidence and prevalence rates are respectively 2-10 and 35-100 per 100,000. There is a slight preponderance in females over males for contracting the disease. UC and CD affect primarily individuals between the ages of 15 and 35 years.

Currently, choice of therapy for IBD depends on pharmacodynamic considerations that govern drug and patient characteristics. Clinical remission (relief of inflammatory symptoms) and mucosal healing are two vital aspects that need to be achieved. Many of the current drugs of choice have a poor correlation between symptomatic relief and mucosal healing. Thus agents that can maintain remission as well as accomplish healing are of particular interest in the management of IBD. In the past decade, several drugs, e.g., conventional salicylates, antibiotics, corticosteroids, immunomodulators, and biological response modifiers, have been used in the management of IBD.

5-Aminosalicylates (5-ASAs): the 5-ASAs (sulfasalazine and the sulfa-free agents) are known to alter the immune response by down-regulating antibody secretion and lymphocyte function, inhibit neutrophil and macrophage chemotaxis and protect intestinal epithelium by enhancing expression of heat shock proteins. In addition, they also inhibit the cyclooxygenase and 5-lipoxygenase pathways of arachidonic acid metabolism that may inhibit the release of chemotactic substances (Grisham, M. B. Lancet, 1994, 344:859-861). 5-ASAs are effective therapeutic agents for mild to moderate conditions of UC. However, 5-ASAs are not the drugs of choice for IBD due to their side effects that may include nausea, allergic reactions and reversible oligospermia.

Antibiotics: historically, antibiotics like metronidazole and quinolones have been used to treat CD, although their effectiveness in ameliorating the condition has not been well documented. The presumed effect of these agents may be in the alteration of the bacterial flora associated with IBD. Antibiotics are not only less effective for IBD but also have associated side effects (anorexia, nausea, rash) and thus may not be the treatment of choice for IBD.

Corticosteroids: corticosteroids have been the oldest of the nonspecific but effective therapeutic regimen used for IBD. Corticosteroids modulate both immunologic and inflammatory responses and inhibit an array of leukocyte functions such as adherence, chemotaxis, phagocytosis arachidocic acid metabolism and eicosanoids production. Although their uses in short-term treatment of CD and UC have been shown, their efficacy in maintenance therapy is far from satisfactory (Munkholm et al., Gut 35: 360-362 (1994)). The failure of corticosteroids in maintenance therapy coupled with the known detrimental side effects of this agent limit their use in the treatment for IBD.

Immunomodulators: the thiopurine agents 6-mercaptoputine (“6-MP”) and azathioprine (“AZA”) have been used in the treatment of CD and UC as steroid sparing agents (Pearson et al., Annals of Internal Medicine 123: 1320142 (1995)). Side effects such as leukopenia, thrombocytopenia associated with these drugs are further complicated by the genetic predisposition of the patient. (Yates et al., Ann. Intern. Med. 126: 608-614 (1997)). Additional side effects such as pancreatitis, hepatitis, nausea and rash are also reported. Methotrexate has been shown to be effective in steroid-dependent CD but not in UC. The side effects of methotrexate include bone marrow suppression, interstitial pneumonitis and neuropathy. Cyclosporine has been effective in the treatment of both CD and UC. Cyclosporine has been particularly shown to be effective in patients with active CD or UC that are resistant or intolerant to corticosteroids (Lichtiger et al., New England Journal of Medicine 330: 1841-1845 (1994)). The side effects of cyclosporin include reversible or irreversible decrease in renal function, hypertension, tremor, and seizure.

Biological Response Modifiers: Infliximab, a chimeric monoclonal IgG1 antibody directed against TNF-a, has been effectively used in the treatment of CD. Although it is effective in maintenance therapy and healing fistulas (Present et al., New England Journal of Medicine 340: 1398-1405 (1999)), side effects include delayed hypersensitivity reactions and lymphoproliferative disorders.

Patients with UC or CD are at risk for several co-morbid conditions. Colon cancer is a well-recognized long-term complication of UC. The risk of developing colon cancer is correlated with duration of disease and extent of involvement, increases progressively after 10 years of disease, and is greatest for pancolitis and distal disease (Hanauer S B. Medical therapy for ulcerative colitis. In: Kirsner J B, editor. Inflammatory Bowel Disease. 5th ed. Philadelphia (PA): W.B. Saunders Company; 1999. pp 529-556). In contrast to sporadic adenocarcinoma of the colon, where the earliest gross lesion is an adenomatous polyp, cancer in patients with UC arises where the flat mucosa develops dysplasia. Cancer risk in the presence of low-grade dysplasia is 10-19% and with high-grade dysplasia is 30-40% (Michelassi F. Indications for surgery in inflammatory bowel disease: the surgeon's perspective. In: Kirsner J B, editor. Inflammatory Bowel Disease. 5th ed. Philadelphia (PA): W.B. Saunders Company; 1999. pp 616-625).

In 1999, approximately 1.7 million people were diagnosed with inflammatory bowel disease. Satisfactory treatment of IBD is an unmet medical need, as existing therapeutics have not been successful in curtailing the disease and preventing surgeries. Up to forty percent of all ulcerative colitis patients undergo surgery, which typically includes the removal of part of the large intestine or a full colostomy. Such surgery is not curative for Crohn's disease, as 75% of all patients undergo at least one surgery in their lifetime, and up to 90% of these patients require additional surgeries. Consequently a therapeutic that can successfully treat inflammatory bowel disease will have the beneficial effects of improving a patient's quality of life, while potentially saving the healthcare system millions of dollars in costs associated with invasive surgical procedures.

2.2 Irritable Bowel Syndrome

Irritable bowel syndrome (“IBS”) is a functional bowel disorder of the gastrointestinal (GI) tract characterized by recurrent abdominal pain and discomfort accompanied by alterations in bowel function, diarrhea, constipation or a combination of both, typically over months or years. A diagnosis of IBS has been reported by 10 to 20% of adults in the United States, and symptoms of IBS are responsible for over 3 million yearly visits to physicians. Research suggests that IBS is one of the most common functional GI disorders. IBS exhibits a predominance in women, with females representing over 70% of IBS sufferers.

Irritable bowel syndrome is generally classified as a “functional” disorder. A functional disorder refers to a disorder or disease where the primary abnormality is an altered physiological function, rather than an identifiable structural or biochemical cause. It characterizes a disorder that generally can not be diagnosed in a traditional way, i.e., as an inflammatory, infectious, or structural abnormality that can be detected by commonly used examination, x-ray, or blood test.

Irritable Bowel Syndrome can be diagnosed based on at least 12 weeks, which need not be consecutive, in the preceding 12 months of abdominal discomfort or pain that has two out of three features: (1) Relieved with defecation; (2) Onset associated with a change in frequency of stool; and/or (3) Onset associated with a change in form (appearance) of stool. Symptoms that cumulatively support the diagnosis of IBS include, but are not limited to, abnormal stool frequency (may be defined as greater than 3 bowel movements per day and less than 3 bowel movements per week); abnormal stool form (lumpy/hard or loose/watery stool); abnormal stool passage (straining, urgency, or feeling of incomplete evacuation); passage of mucus; and bloating or feeling of abdominal distension.

Irritable bowel syndrome is understood as a multi-faceted disorder. In people with IBS, symptoms result from what appears to be a disturbance in the interaction between the gut or intestines, the brain, and the autonomic nervous system that alters regulation of bowel motility or sensory function.

Current pharmaceutical agents that are used in managing IBS include, but are not limited to, laxatives; antidiarrheals (e.g., diphenoxylate (e.g., Lomotil, Lomocot); loperamide (e.g., Imodium, Pepto Diarrhea), cholestyramine (e.g., Questran, Cholybar)); antispasmodics (e.g., dicyclomine, hyoscyamine, and clidinium (in combination with chlordiazepoxide hydrochloride)); peppermint oil; direct smooth muscle relaxants; and antidepressants. Specific serotonin receptors (known as 5-HT3 and 5-HT4) appear to be important in gastrointestinal motility and visceral (internal) sensation. 5HT-3 receptor antagonists (which work by inhibiting action) and 5-HT4 receptor agonists (which work by stimulating action) are also being studied as potential IBS treatments. For example, Alosetron (Lotronex) is a highly selective 5-HT3 antagonist, which has been found to improve overall symptoms and quality of life measurements in women with diarrhea-predominant IBS. Other 5-HT3 antagonists (e.g., cilansetron) are currently under investigation. The 5-HT4 agonists that seem most effective in treating constipation-predominant IBS are tegaserod (Zelnorm/Zelmac) and prucalopride. The M3 receptor antagonists, zamifenacin and darifenacin, are also under investigation as potential treatments for diarrhea-predominant IBS.

2.3 Fibroblast Growth Factors

The fibroblast growth factor (“FGF”) family has more than 20 members, each containing a conserved amino acid core (see, e.g., Powers et al., Endocr. Relat. Cancer, 7 (3): 65-197 (2000)). FGFs regulate diverse cellular functions such as growth, survival, apoptosis, motility, and differentiation (see, e.g., Szebenyi et al., Int. Rev. Cytol., 185: 45-106 (1999)). Members of the FGF family are involved in various physiological and pathological processes during embryogenesis and adult life, including morphogenesis, limb development, tissue repair, inflammation, angiogenesis, and tumor growth and invasion (see, e.g., Powers et al., Endocr. Relat. Cancer, 7 (3): 165-197 (2000); and Szebenyi et al., Int. Rev. Cytol. 185: 45-106 (1999)).

FGFs transduce signals via high affinity interactions with cell surface tyrosine kinase FGF receptors (FGFRs). These FGF receptors are expressed on most types of cells in tissue culture. For example, FGF receptor-1 (FGFR-1), which shows the broadest expression pattern of the four known FGF receptors, contains at least seven tyrosine phosphorylation sites. A number of signal transduction molecules are affected by binding with different affinities to these phosphorylation sites.

FGFs also bind, albeit with low affinity, to heparin sulfate proteoglycans (HSPGs) present on most cell surfaces and extracellular matrices (ECM). Interactions between FGFs and HSPGs may serve to stabilize FGF/FGFR interactions, and to sequester FGFs and protect them from degradation (Szebenyi and Fallon, Int. Rev. Cytol. 185: 45-106. (1999)).

Glia-activating factor (“GAF”), another FGF family member, is a heparin-binding growth factor that was purified from the culture supernatant of a human glioma cell line. See, Miyamoto et al., Mol. Cell Biol. 13 (7): 4251-4259 (1993). GAF shows a spectrum of activity slightly different from those of other known growth factors, and is designated as FGF-9. The human FGF-9 cDNA encodes a polypeptide of 208 amino acids. Sequence similarity to other members of the FGF family was estimated to be around 30%. Two cysteine residues and other consensus sequences found in other family members were also well conserved in the FGF-9 sequence. FGF-9 was found to have no typical signal sequence in its N-terminus like those in acidic FGF and basic FGF.

Acidic FGF and basic FGF are known not to be secreted from cells in a conventional manner. However, FGF-9 was found to be secreted efficiently from cDNA-transfected COS cells despite its lack of a typical signal sequence. It could be detected exclusively in the culture medium of cells. The secreted protein lacked no amino acid residues at the N-terminus with respect to those predicted by the cDNA sequence, except the initiation methionine. The rat FGF-9 cDNA was also cloned, and the structural analysis indicated that the FGF-9 gene is highly conserved.

Through a homology-based genomic mining process, a novel human FGF, FGF-20, was discovered. See U.S. patent application Ser. No. 09/494,585, filed Jan. 13, 2000, and Ser. No. 09/609,543, filed Jul. 3, 2000, the disclosure of each references is incorporated herein by reference. The amino acid sequence of FGF-20 shows close homology with human FGF-9 (70% identity) and FGF-16 (64% identity).

Citation or discussion of a reference herein shall not be construed as an admission that such is prior art to the present invention.

### 3. SUMMARY OF THE INVENTION

In one embodiment, the present invention provides methods of preventing and/or treating a pathology of epithelial cells and/or mesenchymal cells comprising administering to a subject in need thereof a composition comprising one or more CG53135 proteins. In another embodiment, the present invention provides methods of stimulating proliferation, differentiation or migration of epithelial cells and/or mesenchymal cells comprising administering to a subject in need thereof an effective amount of a composition comprising one or more CG53135 proteins.

In a preferred embodiment, the FGF protein in the compositions used in accordance to the present invention is a FGF-20 protein, a fragment, a derivative, a variant, a homolog, or an analog of FGF-20, or a combination thereof. In some embodiments, the FGF protein in the compositions is CG53135-01 (SEQ ID NO:2), CG53135-02 (SEQ ID NO: 4), CG53135-03 (SEQ ID NO:2), CG53135-04 (SEQ ID NO:7), CG53135-05 (SEQ ID NO: 2), CG53135-06 (SEQ ID NO: 10), CG53135-07 (SEQ ID NO:12), CG53135-08 (SEQ ID NO:14), CG53135-09 (SEQ ID NO:16), CG53135-10 (SEQ ID NO:18), CG53135-11 (SEQ ID NO:20), CG53135-12 (SEQ ID NO:22), CG53135-13 (SEQ ID NO:24), CG53135-14 (SEQ ID NO:26), CG53135-15 (SEQ ID NO:28), CG53135-16 (SEQ ID NO:30), CG53135-17 (SEQ ID NO:32), IFC 250059629 (SEQ ID NO:34), IFC 20059669 (SEQ ID NO:36), IFC 317459553 (SEQ ID NO:38), IFC 317459571 (SEQ ID NO:40), IFC 250059596 (SEQ ID NO:10), or IFC316351224 (SEQ ID NO:10), or any two or more combinations of CG53135 proteins. In one embodiment, the FGF proteins in the compositions comprise (1) a protein comprising an amino acid sequence of SEQ ID NO:2, and (2) a protein comprising an amino acid sequence of SEQ ID NO:24. In another embodiment, the FGF proteins in the compositions comprise (1) a protein comprising an amino acid sequence of SEQ ID NO:2, (2) a protein comprising an amino acid sequence of SEQ ID NO:24, (3) a protein comprising an amino acid sequence of SEQ ID NO:26, (4) a protein comprising an amino acid sequence of SEQ ID NO:28, (5) a protein comprising an amino acid sequence of SEQ ID NO:30, and (6) a protein comprising an amino acid sequence of SEQ ID NO:32. In another embodiment, the FGF proteins in the compositions comprise (1) a protein comprising an amino acid sequence of SEQ ID NO:2, (2) a protein comprising an amino acid sequence of SEQ ID NO:24, (3) a protein comprising an amino acid sequence of SEQ ID NO:28, (4) a protein comprising an amino acid sequence of SEQ ID NO:30, and (5) a protein comprising an amino acid sequence of SEQ ID NO:32. In another embodiment, a composition used in accordance to the methods of the invention comprises (1) a protein comprising an amino acid sequence of SEQ ID NO:32; (2) a protein comprising an amino acid sequence of SEQ ID NO:30, (3) a protein comprising an amino acid sequence of SEQ ID NO:28; and (4) a protein comprising an amino acid sequence of SEQ ID. NO:24. In yet another embodiment, the FGF proteins in the compositions comprise (1) a protein comprising an amino acid sequence of SEQ ID NO:2, (2) a protein comprising an amino acid sequence of SEQ ID NO:24, (3) a protein comprising an amino acid sequence of SEQ ID NO:28, (4) a protein comprising an amino acid sequence of SEQ ID NO:30, (5) a protein comprising an amino acid sequence of SEQ ID NO:32, (6) a carbamylated protein comprising an amino acid sequence of SEQ ID NO:24, and (7) a carbamylated protein comprising an amino acid sequence of SEQ ID NO:2.

The present invention provides methods of preventing and/or treating inflammatory bowel disease (“IBD”) (e.g., Crohn's disease, or ulcerative colitis) or irritable bowel syndrome (“IBS”) comprising administering to a subject in need thereof a composition comprising one or more CG53135 proteins.

In one embodiment, the present invention provides a method of preventing or treating inflammatory bowel disease or irritable bowel syndrome comprising administering to a subject in need thereof an effective amount of a composition comprising an isolated protein selected from the group consisting of: (a) a protein comprising an amino acid sequence of SEQ ID NOs:2, 4, 7, 10, 22, 24, 26, 28, 30, 32, 34, 36, 38, or 40; (b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and (c) a fragment of the protein of (a) or (b), which fragment retains cell proliferation stimulatory activity.

In another embodiment, the present invention provides a method of preventing or treating inflammatory bowel disease or irritable bowel syndrome comprising administering to a subject in need thereof an effective amount of a composition comprising a protein isolated from a cultured host cell containing an isolated nucleic acid molecule selected from the group consisting of: (a) a nucleic acid molecule comprising a nucleotide sequence selected from the group consisting of SEQ ID NOs: 3, 5, 6, 8, 9, 21, 23, 25, 27, 29, 31, 33, 35, 37, 39 and 41; (b) a nucleic acid molecule encoding a protein comprising an amino acid sequence selected from the group consisting of SEQ ID NOs: 2, 4, 7, 10, 22, 24, 26, 28, 30, 32, 34, 36, 38, or 40; and (c) a nucleic acid molecule hybridizes under stringent conditions to a nucleotide sequence of SEQ ID NO: 3, 5, 6, 8, 9, 21, 23, 25, 27, 29, 31, 33, 35, 37, 39 or 41, or a complement of said nucleic acid molecule. In some embodiments, the host cell is a eukaryotic cell. In some embodiments, the host cell is a prokaryotic cell. In a preferred embodiment, the host cell is E. coli. In one embodiment, the protein isolated from a cultured host cell has a purity of at least 80%, at least 85%, at least 90%, at least 95%, or at least 98%.

The present invention also provides methods of preventing and/or treating IBD or IBS comprising administering a composition comprising a pharmaceutically acceptable carrier and one or more CG53135 proteins.

In one embodiment, the present invention provides a method of preventing and/or treating IBD or IBS comprising administering to a subject in need thereof a composition comprising 0.02-0.2 M acetate, 0.5-5% glycerol, 0.2-0.5 M arginine-HCl, and a CG53135 protein. In a specific embodiment, the present invention provides a method of preventing and/or treating IBD comprising administering to a subject in need thereof a composition comprising 0.04M acetate, 3% Glycerol (volume/volume), 0.2M Arginine-HCl at pH 5.3, and 0.005-5 mg/ml (preferably 0.8 mg/ml) of a CG53135 protein.

In another embodiment, the present invention provides a method of preventing and/or treating IBD comprising administering to a subject in need thereof a composition comprising 0.01-1 M arginine in a salt form, sulfobutyl ether Beta-cyclodextrin sodium, or sucrose, about 0.01-0.1 M sodium phosphate monobasic (NaH₂PO₄.H₂O), about 0.01%-0.1% weight/volume (“w/v”) polysorbate 80 or polysorbate 20, and a CG53135 protein (preferably about 0.005 mg/ml to about 50 mg/ml). The arginine in a salt form can be selected from the group consisting of arginine, arginine sulfate, arginine phosphate, and arginine hydrochloride. Preferably, the arginine in a salt form, sulfobutyl ether Beta-cyclodextrin sodium or sucrose in the compositions of the invention is of 0.01-0.7 M. More preferably, a composition of the invention comprises an arginine in a salt form at a concentration of 0.5 M. In one embodiment, the sodium phosphate monobasic in the compositions of the invention is 0.05 M. In another embodiment, the polysorbate 80 or polysorbate 20 in the compositions of the invention is 0.01% (w/v). In yet another embodiment, one or more CG53135 proteins in the compositions of the invention are at a concentration of 5-30 mg/ml. In a specific embodiment, one or more CG53135 proteins in the compositions of the invention are 10 mg/ml. In some embodiment, the compositions comprising one or more CG53135 proteins that are used in accordance to the present invention are lyophilized or spray dried.

The present invention further provides methods of stimulating proliferation, differentiation, or migration of epithelial cells or mesenchymal cells comprising administering to a subject in need thereof an effective amount of a composition comprising one or more CG53135 proteins. In one embodiment, the present invention provides a method of stimulating proliferation, differentiation or migration of epithelial cells or mesenchymal cells comprising administering to a subject in need thereof an effective amount of a composition comprising an isolated protein selected from the group consisting of: (a) a protein comprising an amino acid sequence of SEQ ID NOs:2, 4, 7, 10, 22, 24, 26, 28, 30, 32, 34, 36, 38, or 40; (b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and (c) a fragment of the protein of (a) or (b), which fragment retains cell proliferation stimulatory activity. In one embodiment, the epithelial cells or mesenchymal cells are located at the alimentary tract of said subject. In another embodiment, the epithelial cells or mesenchymal cells are located at the pulmonary tract of said subject. In a specific embodiment, the epithelial cells or mesenchymal cells are located at trachea.

Pharmaceutical compositions and kits are also provided by the present invention.

3.1 Terminology

As used herein, the term “CG53135”, refers to a class of proteins (including peptides and polypeptides) or nucleic acids encoding such proteins or their complementary strands, where the proteins comprise an amino acid sequence of SEQ ID NO:2 (211 amino acids), or its fragments, derivatives, variants, homologs, or analogs. In a preferred embodiment, a CG53135 protein retains at least some biological activity of FGF-20. As used herein, the term “biological activity” means that a CG53135 protein possesses some but not necessarily all the same properties of (and not necessarily to the same degree as) FGF-20.

A member (e.g., a protein and/or a nucleic acid encoding the protein) of the CG53135 family may further be given an identification name. For example, CG53135-01 (SEQ ID NOs:1 and 2) represents the first identified FGF-20 (see U.S. patent application Ser. No. 09/494,585); CG53135-05 (SEQ ID NOs:8 and 2) represents a codon-optimized, full length FGF-20 (i.e., the nucleic acid sequence encoding FGF-20 has been codon optimized, but the amino acid sequence has not been changed from the originally identified FGF-20); CG53135-12 (SEQ ID NOs:21 and 22) represent a single nucleotide polymorphism (“SNP”) of FGF-20 where one amino acid in CG53135-12 is different from SEQ ID NO:2 (the aspartic acid at position 206 is changed to asparagine, “²⁰⁶D? N”). Some members of the CG53135 family may differ in their nucleic acid sequences but encode the same CG53135 protein, e.g., CG53135-01, CG53135-03, and CG53135-05 all encode the same CG53135 protein. An identification name may also be an in-frame clone (“IFC”) number, for example, IFC 250059629 (SEQ ID NOs:33 and 34) represents amino acids 63-196 of the full length FGF-20 (cloned in frame in a vector). Table 1 shows a summary of some of the CG53135 family members. In one embodiment, the invention includes a variant of FGF-20 protein, in which some amino acids residues, e.g., no more than 1%, 2%, 3%, 5%, 10% or 15% of the amino acid sequence of FGF-20 (SEQ ID NO:2), are changed. In another embodiment, the invention includes nucleic acid molecules that can hybridize to FGF-20 under stringent hybridization conditions.

TABLE 1
Summary of some of the CG53135 family members

| | SEQ ID NO | |
|---|---|---|
| Name | (DNA/Protein) | Brief Description |
| CG53135-01 | 1 and 2 | FGF-20 wild type, stop codon removed |
| CG53135-02 | 3 and 4 | Codon optimized, amino acids 2-54 |
| | | (as numbered in SEQ ID NO: 2) were |
| | | removed |
| CG53135-03 | 5 and 2 | FGF-20 wild type |
| CG53135-04 | 6 and 7 | Amino acids 20-51 (as numbered in |
| | | SEQ ID NO: 2) were removed, also |
| | | valine at position 85 is changed to |
| | | alanine (“⁸⁵V? A”) |
| CG53135-05 | 8 and 2 | Codon optimized, full length FGF-20 |
| CG53135-06 | 9 and 10 | Amino acids 20-51 (as numbered in |
| | | SEQ ID NO: 2) were removed |
| CG53135-07 | 11 and 12 | Protein consisting of amino acids |
| | | 1-18 (as numbered in SEQ ID |
| | | NO: 2) |
| CG53135-08 | 13 and 14 | Protein consisting of amino acids |
| | | 32-52 (as numbered in SEQ ID |
| | | NO: 2) |
| CG53135-09 | 15 and 16 | Protein consisting of amino acids |
| | | 173-183 (as numbered in SEQ |
| | | ID NO: 2) |
| CG53135-10 | 17 and 18 | Protein consisting of amino acids |
| | | 192-211 (as numbered in SEQ |
| | | ID NO: 2) |
| CG53135-11 | 19 and 20 | Protein consisting of amino acids |
| | | 121-137 (as numbered in SEQ |
| | | ID NO: 2) |
| CG53135-12 | 21 and 22 | FGF-20 SNP, aspartic acid at position |
| | | 206 is changed to asparagines |
| | | (“²⁰⁶D? N”) as compared |
| | | to CG53135-01 |
| CG53135-13 | 23 and 24 | CG53135-05 minus first 2 amino acids |
| | | at the N-terminus |
| CG53135-14 | 25 and 26 | CG53135-05 minus first 8 amino acids |
| | | at the N-terminus |
| CG53135-15 | 27 and 28 | CG53135-05 minus first 11 amino acids |
| | | at the N-terminus |
| CG53135-16 | 29 and 30 | CG53135-05 minus first 14 amino acids |
| | | at the N-terminus |
| CG53135-17 | 31 and 32 | CG53135-05 minus first 23 amino acids |
| | | at the N-terminus |
| IFC 250059629 | 33 and 34 | In frame clone, open reading frame |
| | | comprising a nucleotide sequence |
| | | encoding amino acids 63-196 of |
| | | FGF-20 (SEQ ID NO: 2) |
| IFC 250059669 | 35 and 36 | In frame clone, open reading frame |
| | | comprising a nucleotide sequence |
| | | encoding amino acids 63-211 of |
| | | FGF-20 (SEQ ID NO: 2) |
| IFC 317459553 | 37 and 38 | In frame clone, open reading frame |
| | | comprising a nucleotide sequence |
| | | encoding amino acids 63-194 of |
| | | FGF-20 (SEQ ID NO: 2) with ¹⁵⁹G? E |
| IFC 317459571 | 39 and 40 | In frame clone, open reading frame |
| | | comprising a nucleotide sequence |
| | | encoding amino acids 63-194 of |
| | | FGF-20 (SEQ ID NO: 2) |
| IFC 250059596 | 41 and 10 | In frame clone, open reading frame |
| | | comprising a nucleotide sequence |
| | | encoding amino acids 1-19 and |
| | | 52-211 of FGF-20 (SEQ ID NO: 2) |
| IFC 316351224 | 41 and 10 | In frame clone, open reading frame |
| | | comprising a nucleotide sequence |
| | | encoding amino acids 1-19 and |
| | | 52-211 of FGF-20 (SEQ ID NO: 2). |

As used herein, the term “effective amount” refers to the amount of a therapy (e.g., a composition comprising a CG53135 protein) which is sufficient to reduce and/or ameliorate the severity and/or duration of inflammatory bowel disease or one or more symptoms thereof, prevent the advancement of inflammatory bowel disease, cause regression of inflammatory bowel disease, prevent the recurrence, development, or onset of one or more symptoms associated with inflammatory bowel disease, or enhance or improve the prophylactic or therapeutic effect(s) of another therapy.

As used herein, the term “FGF-20” refers to a protein comprising an amino acid sequence of SEQ ID NO:2, or a nucleic acid sequence encoding such a protein and/or a complementary strand thereof.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non limiting example, stringent hybridization conditions comprise a salt concentration from about 0.1 M to about 1.0 M sodium ion, a pH from about 7.0 to about 8.3, a temperature is at least about 60° C., and at least one wash in 0.2×SSC, 0.01% BSA. In another non-limiting example, stringent hybridization conditions are hybridization at 6× sodium chloride/sodium citrate (SSC) at about 45° C., followed by one or more washes in 0.1×SSC, 0.2% SDS at about 68° C. In yet another non-limiting example, stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “isolated” in the context of a protein agent refers to a protein agent that is substantially free of cellular material or contaminating proteins from the cell or tissue source from which it is derived, or substantially free of chemical precursors or other chemicals when chemically synthesized. The language “substantially free of cellular material” includes preparations of a protein agent in which the protein agent is separated from cellular components of the cells from which it is isolated or recombinantly produced. Thus, a protein agent that is substantially free of cellular material includes preparations of a protein agent having less than about 30%, 20%, 10%, or 5% (by dry weight) of host cell proteins (also referred to as a “contaminating proteins”). When the protein agent is recombinantly produced, it is also preferably substantially free of culture medium, i.e., culture medium represents less than about 20%, 10%, or 5% of the volume of the protein agent preparation. When the protein agent is produced by chemical synthesis, it is preferably substantially free of chemical precursors or other chemicals, i.e., it is separated from chemical precursors or other chemicals that are involved in the synthesis of the protein agent. Accordingly, such preparations of a protein agent have less than about 30%, 20%, 10%, 5% (by dry weight) of chemical precursors or compounds other than the protein agent of interest. In a specific embodiment, protein agents disclosed herein are isolated.

As used herein, the term “isolated” in the context of nucleic acid molecules refers to a nucleic acid molecule that is separated from other nucleic acid molecules that are present in the natural source of the nucleic acid molecule. Moreover, an “isolated” nucleic acid molecule, such as a cDNA molecule, can be substantially free of other cellular material or culture medium when produced by recombinant techniques, or substantially free of chemical precursors or other chemicals when chemically synthesized. In a specific embodiment, nucleic acid molecules are isolated.

As used herein, the term “in combination” refers to the use of more than one therapy. The use of the term “in combination” does not restrict the order in which therapies are administered to a subject in need thereof. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject in need thereof.

As used herein, the terms “prevent,” “preventing,” and “prevention” refer to the prevention of the recurrence, onset, or development of inflammatory bowel disease or one or more symptoms thereof in a subject resulting from the administration of a therapy (e.g., a composition comprising a CG53135 protein), or the administration of a combination of therapies.

As used herein, the term “prophylactically effective amount” refers to the amount of a therapy (e.g., a composition comprising a CG53135 protein) which is sufficient to result in the prevention of the development, recurrence, or onset of inflammatory bowel disease or one or more symptoms thereof, or to enhance or improve the prophylactic effect(s) of another therapy.

As used herein, the terms “subject” and “subjects” refer to an animal, preferably a mammal, including a non-primate (e.g., a cow, pig, horse, cat, or dog), a primate (e.g., a monkey, chimpanzee, or human), and more preferably a human. In a certain embodiment, the subject is a mammal, preferably a human, who has been exposed to or is going to be exposed to an insult affecting rapidly proliferating tissues (such as radiation, chemotherapy, or chemical warfare agents). In another embodiment, the subject is a farm animal (e.g., a horse, pig, or cow) or a pet (e.g., a dog or cat) that has been exposed to or is going to be exposed to a similar insult. The term “subject” is used interchangeably with “patient” in the present invention.

As used herein, the terms “treat,” “treatment,” and “treating” refer to the reduction of the progression, severity, and/or duration of inflammatory bowel disease or amelioration of one or more symptoms thereof, wherein such reduction and/or amelioration result from the administration of one or more therapies (e.g., a composition comprising a CG53135 protein).

As used herein, the term “therapeutically effective amount” refers to the amount of a therapy (e.g., a composition comprising a CG53135 protein), which is sufficient to reduce the severity of inflammatory bowel disease, reduce the duration of inflammatory bowel disease, prevent the advancement of inflammatory bowel disease, cause regression of inflammatory bowel disease, ameliorate one or more symptoms associated with inflammatory bowel disease, or enhance or improve the therapeutic effect(s) of another therapy.

### 4. BRIEF DESCRIPTION OF THE DRAWINGS

FIG. 1. RP-HPLC analysis of CG53135-05 E. coli purified product (by Process 1 and 2, respectively, see Section 6.16.1 and 6.16.2 for description of the processes).

FIG. 2. Tryptic map of CG53135-05 E. coli purified product (by Process 1 and 2, respectively).

FIG. 3 provides the results of a dose titration growth experiment carried out using 786-0 human renal carcinoma cells. In this experiment incorporation of bromodeoxyuridine induced by varying amounts of CG53135-01 E. coli purified product (designated in FIG. 3 as 20858) was determined.

FIG. 4 displays the biological activity of a truncated form of recombinant CG53135-02 (denoted by (d1-23) FGF20 in the FIG.) as represented by its effects on DNA synthesis, compared to that of full length CG53135-01 (denoted FGF20 in the FIG.). NIH 3T3 mouse fibroblasts were serum-starved, incubated with the indicated factor for 18 hours, and analyzed by a BrdU incorporation assay.

FIG. 5 shows the results of experiments assessing the receptor binding specificity of CG53135. NIH 3T3 cells were serum-starved, incubated with the indicated growth factor (square=PDGF-BB; triangle=aFGF; circle=FGF-CX) either alone or together with the indicated soluble FGFR, and analyzed by a BrdU incorporation assay. Experiments were performed in triplicate and are represented as the percent BrdU increase in incorporation of BrdU relative to cells receiving the growth factor alone.

FIG. 6. Effect of CG53135 in in vitro wound repair in CaCo2, HT-29, IEC-6 human cell lines.

FIG. 7. Effect of CG53135 on COX-2 gene expression in HT-29 cells. RT-PCR analysis was carried out to detect the expression of COX-2 gene in HT-29 cell line, in the presence of various concentration of CG53135 (0.1, 1.0, 10, 100 ng/ml). COX-2 expression was also analyzed at various time points (1, 3, 6, 24 hrs) after the addition of 10 ng/ml of CG53135.

FIG. 8. Effect of CG53135 on COX-2 gene expression in Caco2 cells. RT-PCR analysis was carried out to detect the expression of COX-2 gene in Caco2 cell line, in the presence of various concentration of CG53135 (0.1, 1.0, 10, 100 ng/ml). COX-2 expression was also analyzed at various time points (1, 3, 6, 24 hrs) after the addition of 100 ng/ml of CG53135.

FIG. 9. Effect of CG53135 on COX-2 gene expression in IEC-6 cells. RT-PCR analysis was carried out to detect the expression of COX-2 gene in IEC-6 cell line, in the presence of various concentration of CG53135 (0.1, 1.0, 10, 100 ng/ml). COX-2 expression was also analyzed at various time points (1, 3, 6, 24 hrs) after the addition of 100 ng/ml of CG53135.

FIG. 10. Effect of CG53135 on ITF gene expression in HT-29 and Caco2 cells. ITF gene was detected by mRNA expression in HT-29 and Caco2 cells, in the presence of various concentration of CG53135 (0.1, 1.0, 10, 100 ng/ml). ITF gene expression was also analyzed at various time points (1, 3, 6, 24 hrs) after the addition of 100 ng/ml of CG53135.

FIG. 11. Effect of CG53135 in inducing COX-2, TGF-β, ITF, PPAR-? in HT-29 mRNA expression analysis revealed that, upon induction with 100 ng/ml of CG53135 for 48 hours, mammalian cells expressed COX-2, TGF-β, ITF, PPAR-? genes.

FIG. 12. Mechanism of Epithelial Restitution by CG53135. To assess whether TGF-β mediates epithelial restitution by FGF-20, wound repair test was performed. Caco2 cells were incubated with CG53135-01 E. coli purified product (100 ng/ml) and anti TGF-β (20 μg/ml) and percent closure was measured.

FIG. 13. Effect of CG53135 in stimulation of kinases in Caco2 cells. Expression of signal transducing kinases was analyzed after incubation of Caco2 cells with CG53135 E. coli purified product (100 ng/ml) for different time points (10, 30, 60 mins).

FIG. 14. Effect of kinase inhibitors in the expression of COX-2 gene in Caco2 cells. Caco2 cells were incubated with CG53135-01 E. coli purified product (100 ng/ml) in the presence of 40 μM of PD098059 and 20 μM of SB203580 and COX-2 expression was analyzed.

FIG. 15. Effect of CG53135 in stimulation of kinases in THP-1 cells. Macrophage cell line, THP-1 was cultured with CG53135-01 E. coli purified product (100 ng/ml) for various time periods. Expression of signal transducing kinases was analyzed at different time points (10, 30, 60 mins).

FIG. 16. Effect of CG53135 on expression of kinases in intestinal epithelial cells. Caco2 cells were incubated with FGF-20 (100 ng/ml) for 10, 30, 60 minutes and expression of p-Elk-1, p-ATF-2 and p-PKC was analyzed. Similarly, HT-29 cells were incubated with FGF-20 (100 ng/ml) for 10, 30, 60 minutes and expression of C-Fos and C-Jun was analyzed.

FIG. 17. Effect of CG53135 in activating ITF Transcription in HT-29 cells. ITF promoter activity in HT-29 cells was measured by reporter assay in the presence of FGF-20 at a concentration of 100 ng/ml. FIG. 17 also shows ITF expression in HT-29 cells in the presence of CG53135-01 E. coli purified product.

FIG. 18. Effect of NFkB on activation of CG53135 and stimulation of COX-2 in intestinal epithelial cells. NFkB promoter activity was assayed in HT-29 and Caco2 cell lines in the presence of CG53135-01 E. coli purified product. FIG. 18 also shows expression of COX-2 Caco2 cells, in the presence of MG132, which is a proteosome inhibitor.

FIG. 19. Dose Response of CG53135-induced DNA synthesis in NIH 3T3 Fibroblasts. Serum starved NIH 3T3 cells were treated with purified CG53135-01 (CG53135 in figure), 10% serum or vehicle only (control). DNA synthesis was measured in triplicate for each sample, using a BrdU incorporation assay. Data points represent average BrdU incorporation and bars represent standard error (SE).

FIG. 20. CG53135 stimulates Growth of NIH 3T3 Fibroblasts. Duplicate wells of serum starved NIH 3T3 cells were treated for 1 day with purified CG53135-01 (1 ug) or vehicle control. Cell counts for each well were determined in duplicate. Y— axis identifies cell number, which is the average of 4 cell counts (treatment duplicates×duplicate counts) and standard error (SE).

FIG. 21. CG53135 induces DNA synthesis in 786-0 Kidney Epithelial cells. Serum starved 786-0 cells were left untreated or treated with partially purified CG53135-01 (from 5 ng/uL stock), or with vehicle control (mock). DNA synthesis was measured in triplicate for each sample, using a BrdU incorporation assay. Data points represent average BrdU incorporation and bars represent standard error (SE).

FIG. 22 presents bar graphs representing mean body weights of mice on day 0, and on day 6 after various treatments.

FIG. 23 presents bar graphs representing changes in mean body weights of mice between day 0 and day 6 after various treatments.

FIG. 24 presents bar graphs representing percent changes in mean body weights of mice between day 0 and day 6 after various treatments.

FIG. 25 presents bar graphs representing mean colon blood content scores in mice after various treatments.

FIG. 26 presents bar graphs representing changes in mean weights of the spleens of mice between day 0 and day 6 after various treatments.

FIG. 27 presents bar graphs representing changes in mean spleen weights of mice between day 0 and day 6 after various treatments.

FIG. 28 presents bar graphs representing changes in mean colon weights of mice between day 0 and day 6 after various treatments.

FIG. 29 presents bar graphs representing changes in mean colon weights of mice between day 0 and day 6 after various treatments.

FIG. 30 presents bar graphs representing percent changes in mean colon weights of mice between day 0 and day 6 after various treatments.

FIG. 31 presents bar graphs representing changes in mean colon lengths of mice between day 0 and day 6 after various treatments.

FIG. 32 presents bar graphs representing percent changes in mean colon lengths of mice between day 0 and day 6 after various treatments.

FIG. 33 presents bar graphs representing mean colon edema scores in mice after various treatments.

FIG. 34 presents bar graphs representing mean colon inflammation scores in mice after various treatments.

FIG. 35 presents bar graphs representing mean colon epithelial loss scores in mice after various treatments.

FIG. 36 presents bar graphs representing mean colon erosion content scores in mice after various treatments.

FIG. 37 presents bar graphs representing sum of histopathology scores in mice after various treatments.

FIG. 38 presents bar graphs representing histopathology score differences in mice after various treatments.

FIG. 39 presents bar graphs representing mean splenic lymphoid atrophy scores in mice after various treatments.

FIG. 40 presents photomicrographs at 400× in the original image of mouse colon cross sections. Panel A, DSS plus Vehicle; Panel B, DSS+AB020858; Panel C, Normal mouse.

FIG. 41 presents photomicrographs at 50× in the original image of mouse colon crossections. Panel A, DSS plus Vehicle; Panel B, DSS+AB020858; Panel C, Normal mouse.

FIG. 42 presents the change in mean body weight from day 0 upon treating mice with varying doses of AB020258 (CG53135).

FIG. 43 presents the percent change in mean body weight from day 0 upon treating mice with varying doses of AB020258.

FIG. 44 presents mean colon blood content score upon treating mice with varying doses of AB020258.

FIG. 45 presents mean colon lengths upon treating mice with varying doses of AB020258.

FIG. 46 presents mean colon lengths as a percent of normal, upon treating mice with varying doses of AB020258.

FIG. 47 presents mean colon weights upon treating mice with varying doses of AB020258.

FIG. 48 presents mean colon weights as a percent of normal, upon treating mice with varying doses of AB020258.

FIG. 49 presents mean spleen weights upon treating mice with varying doses of AB020258.

FIG. 50 presents mean distal colon inflammation score upon treating mice with varying doses of AB020258.

FIG. 51 presents mean distal colon gland loss score upon treating mice with varying doses of AB020258.

FIG. 52 presents mean distal colon erosion score upon treating mice with varying doses of AB020258.

FIG. 53 presents mean sums of histopathology scores upon treating mice with varying doses of AB020258.

FIG. 54 presents mean splenic lymphoid atrophy score upon treating mice with varying doses of AB020258.

FIG. 55 presents mean splenic extramedullary hematopoiesis score upon treating mice with varying doses of AB020258.

FIG. 56 presents the effect of CG53135 Treatment on Weight Loss in Indomethacin-treated rats. Body weight change from Day 0 to Day 5 is shown in grams.

FIG. 57 presents the effect of CG53135 Treatment on Small Intestine Weight in Indomethacin-treated rats.

FIG. 58 presents effect of CG53135 Treatment on absolute neutrophil and lymphocyte counts in indomethacin-treated rats. Blood was collected on Day 5 at necropsy and the cell counts were determined.

FIG. 59 presents effect of CG53135 Treatment on Histopathology Scores in Indomethacin-treated rats. Five sections of affected intestine were evaluated and scored for necrosis and inflammation as described in the methods.

FIG. 60 presents images showing the protective effect of CG53135 on intestinal architecture. Panel A: Small intestine from normal control animal treated iv with vehicle (BSA). Panel. B: Small intestine from indomethacin-treated rat, further treated with vehicle (BSA) iv. Panel C: Small intestine from indomethacin-treated rat further treated with CG53135, 0.2 mg/kg iv. Sections were stained with H&E and visualized at a magnification of 25). FIG. 60 shows the protective Effect of CG53135 on Intestinal Architecture in indomethacin treated rats. Panel A, normal control; Panel B, disease control (indomethacin treated); Panel C, disease model animal treated with 0.2 mg/kg iv CG53135. Photomicrographs were obtained on sections stained with hemotoxylin and eosin, at 25× magnification.

FIG. 61 shows the effect of CG53135 treatment on BrdU Labeling in the Intestine. BrdU incorporation was detected by Immunoperoxidase staining. Panel A: Small intestine from normal control animal (100×). Panel B: Small intestine from indomethacin+vehicle (BSA) treated animal (50×). Panel C: Small intestine from indomethacin+CG53135 0.2 mg/kg iv treated rat (50×).

FIG. 62. Effect of therapeutically-administered CG53135 on survival in the DSS model of colitis. Female Balb/c mice were exposed to 4% DSS in drinking water for 7 days (day 0 to day 6) and then switched to normal drinking water for 4 additional days (day 7 to day 10). CG53135 is identified as FGF-20 in FIG. 64. Disease control animals (n=9) received daily SC injections of vehicle solution on day 4 to day 9. CG53135 groups (n=9) received daily SC injections of the indicated concentrations of CG53135 on day 4 to day 9. Normal control animals (n=3) were not exposed to DSS, but did receive daily SC injections of vehicle solution on day 4 to day 9. Animal survival was recorded on a daily basis and the experiment was concluded on day 10. Note that the disease control and the 0.2 mg/kg CG53135 groups overlap.

FIG. 63(A) Weight Change and histopathology in prophylactic group (IL-10KO mice). IL-10 KO mice were treated with various concentrations of CG53135 E. coli purified product (0.2, 1, 5 mg/kg) and weight change and histopathology was assessed. (B) Total Cecal Histologic Score in prophylactic group (IL-10 KO mice). IL-10 KO mice were treated with various concentrations of FGF-20 (0.2, 1, 5 mg/kg) and total cecal histology was scored as described as described in Example 38.

FIG. 64(A) IL-12 production in prophylactic group. IL-12 production was assayed by ELISA as described in Example 40, in MLN and colonic strip culture established. (B) IFN-? production in prophylactic group. IFN-? production was assayed by ELISA, in MLN and colonic strip culture established. (C) PGE2 production in prophylactic group. PGE2 production was assayed by ELISA in MLN prepared.

FIG. 65. FACS analysis (prophylactic group). FACS analysis was performed to get the total MLN number as well as number of CD4+, CD8+ and CD4+ CD69+cells.

FIG. 66. Weight change in treatment group. Weight change in the treatment group was assessed.

FIG. 67. Histology of Cecum (treatment) was analyzed in vehicle control as well as CG53135 treated animals.

FIG. 68. Histology of Rectum (treatment) was analyzed in vehicle control as well as CG53135 treated animals.

FIG. 69. Total Cecal Histologic Score in treatment group (IL-10 KO mice). IL-10 KO mice were treated with CG53135 E. coli purified product (5 mg/kg) and total cecal histology was scored.

FIG. 70. IL-12 production in treatment group. IL-12 production was assayed by ELISA in gut culture and unseparated splenocytes of CG53135 treated IL-10 KO mice.

FIG. 71. IFN-? production in treatment group. IFN-? production was assayed by ELISA in gut culture and unseparated splenocytes of CG53135 treated IL-10KO mice.

FIG. 72. PGE2 and TNF-a production in treatment group. PGE2 and TNF-a production was assayed by ELISA, gut culture and unseparated splenocytes of CG53135 treated IL-10 KO mice.

FIG. 73 shows FACS analysis of MLN number, CD4+ and CD8+ and CD69+cells, all of which were decreased in CG53135 treated group as compared to the vehicle treatment.

FIG. 74. Effect of CG53135 in inducing COX-2, IL-10, ITF, TGF-Lgene expression in Colonic Tissue and unseparated Mesenteric Lymph Node (MLN) cells in C57BL6 wild type mice. RT-PCR analysis was carried out in colonic tissue and unseparated MLN extracted from WT mice treated with CG53135 (5 mg/kg) for 7 days. Expression of various protective genes like COX-2, IL-10, ITF, TGF-β was analyzed.

### 5. DETAILED DESCRIPTION OF THE INVENTION

The present invention provides methods for the prevention and/or treatment of inflammatory bowel disease. In particular, the present invention provides Fibroblast Growth Factor (FGF) 20, its variants, derivatives, homologs, and analogs (collectively referred to as “CG53135”) that can be used in the treatment and/or prevention of inflammatory bowel disease.

For clarity of disclosure, and not by way of limitation, the detailed description of the invention is divided into the following subsections:
(i) CG53135
(ii) Methods of Preparing CG53135
(iii) Characterization and Demonstration of CG53135 Activities and Monitoring Effects During Treatment
(iv) Prophylactic and Therapeutic Uses
(v) Pharmaceutical Compositions

5.1 CG53135

The present invention provides methods of prevention and/or treatment inflammatory bowel disease or irritable bowel syndrome comprising administering to a subject in need thereof a composition comprising one or more CG53135 proteins. As used herein, the term “CG53135” refers to a class of proteins (including peptides and polypeptides) or nucleic acids encoding such proteins or their complementary strands, where the proteins comprise an amino acid sequence of SEQ ID NO:2 (211 amino acids), or its fragments, derivatives, variants, homologs, or analogs.

In one embodiment, a CG53135 protein is a variant of FGF-20. It will be appreciated by those skilled in the art that DNA sequence polymorphisms that lead to changes in the amino acid sequences of the FGF-20 protein may exist within a population (e.g., the human population). Such genetic polymorphism in the FGF-20 gene may exist among individuals within a population due to natural allelic variation. Such natural allelic variations can typically result in 1-5% variance in the nucleotide sequence of the FGF-20 gene. Any and all such nucleotide variations and resulting amino acid polymorphisms in the FGF-20 protein, which are the result of natural allelic variation of the FGF-20 protein, are intended to be within the scope of the invention. In one embodiment, a CG53135 is CG53135-12 (SEQ ID NOs:21 and 22), which is a single nucleotide polymorphism (“SNP”) of FGF-20 (i.e., ²⁰⁶D? N). (For more detailed description of CG53135-12, see e.g., U.S. patent application Ser. No. 10/702,126, filed Nov. 4, 2003, the disclosure of which is incorporated herein by reference in its entirety.) Additional examples of FGF-20 SNPs can be found in Section 6.1, infra.

In another embodiment, CG53135 refers to a nucleic acid molecule encoding a FGF-20 protein from other species or the protein encoded thereby, and thus has a nucleotide or amino acid sequence that differs from the human sequence of FGF-20. Nucleic acid molecules corresponding to natural allelic variants and homologues of the FGF-20 cDNAs of the invention can be isolated based on their homology to the human FGF-20 nucleic acids disclosed herein using the human cDNAs, or a portion thereof, as a hybridization probe according to standard hybridization techniques under stringent hybridization conditions.

In another embodiment, CG53135 refers to a fragment of an FGF-20 protein, including fragments of variant FGF-20 proteins, mature FGF-20 proteins, and variants of mature FGF-20 proteins, as well as FGF-20 proteins encoded by allelic variants and single nucleotide polymorphisms of FGF-20 nucleic acids. An example of an FGF-20 protein fragment includes, but is not limited to, residues 2-211, 3-211, 9-211, 12-211, 15-211, 24-211, 54-211, or 55-211 of FGF-20 (SEQ ID NO:2). In one embodiment, CG53135 refers to a nucleic acid encodes a protein fragment that includes residues 2-211, 3-211, 9-211, 12-211, 15-211, 24-211, 54-211, or 55-211 of SEQ ID NO:2.

The invention also encompasses derivatives and analogs of FGF-20. The production and use of derivatives and analogs related to FGF-20 are within the scope of the present invention.

In a specific embodiment, the derivative or analog is functionally active, i.e., capable of exhibiting one or more functional activities associated with a full-length, wild-type FGF-20. Derivatives or analogs of FGF-20 can be tested for the desired activity by procedures known in the art, including but not limited to, using appropriate cell lines, animal models, and clinical trials.

In particular, FGF-20 derivatives can be made via altering FGF-20 sequences by substitutions, insertions or deletions that provide for functionally equivalent molecules. In one embodiment, such alteration of an FGF-20 sequence is done in a region that is not conserved in the FGF protein family. Due to the degeneracy of nucleotide coding sequences, other DNA sequences which encode substantially the same amino acid sequence as FGF-20 may be used in the practice of the present invention. These include, but are not limited to, nucleic acid sequences comprising all or portions of FGF-20 that are altered by the substitution of different codons that encode a functionally equivalent amino acid residue within the sequence, thus producing a silent change. In a preferred embodiment, a wild-type FGF-20 nucleic acid sequence is codon-optimized to the nucleic acid sequence of SEQ ID NO:8 (CG53135-05). Likewise, the FGF-20 derivatives of the invention include, but are not limited to, those containing, as a primary amino acid sequence, all or part of the amino acid sequence of FGF-20 including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. FGF-20 derivatives of the invention also include, but are not limited to, those containing, as a primary amino acid sequence, all or part of the amino acid sequence of FGF-20 including altered sequences in which amino acid residues are substituted for residues with similar chemical properties. In a specific embodiment, 1, 2, 3, 4, or 5 amino acids are substituted.

Derivatives or analogs of FGF-20 include, but are not limited to, those proteins which are substantially homologous to FGF-20 or fragments thereof, or whose encoding nucleic acid is capable of hybridizing to the FGF-20 nucleic acid sequence.

The FGF-20 derivatives and analogs of the invention can be produced by various methods known in the art. The manipulations that result in their production can occur at the gene or protein level. For example, the cloned FGF-20 gene sequence can be modified by any of numerous strategies known in the art (e.g., Maniatis, T., 1989, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the gene encoding a derivative or analog of FGF-20, care should be taken to ensure that the modified gene remains within the same translational reading frame as FGF-20, uninterrupted by translational stop signals, in the gene region where the desired FGF-20 activity is encoded.

Additionally, the FGF-20-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C. et al., 1978, J. Biol. Chem 253: 6551), use of TAB.RTM. linkers (Pharmacia), etc.

Manipulations of the FGF-20 sequence may also be made at the protein level. Included within the scope of the invention are FGF-20 fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, reagents useful for protection or modification of free NH2— groups, free COOH— groups, OH— groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH4; acetylation, formylation, oxidation, reduction; metabolic synthesis in the presence of tunicamycin; etc.

In addition, analogs and derivatives of FGF-20 can be chemically synthesized. For example, a protein corresponding to a portion of FGF-20 which comprises the desired domain, or which mediates the desired aggregation activity in vitro, or binding to a receptor, can be synthesized by use of a peptide synthesizer. Furthermore, if desired, nonclassical amino acids or chemical amino acid analogs can be introduced as a substitution or addition into the FGF-20 sequence. Non-classical amino acids include, but are not limited to, the D-isomers of the common amino acids, a-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Ca-methyl amino acids, and Na-methyl amino acids.

In a specific embodiment, the FGF-20 derivative is a chimeric or fusion protein comprising FGF-20 or a fragment thereof fused via a peptide bond at its amino- and/or carboxy-terminus to a non-FGF-20 amino acid sequence. In one embodiment, the non-FGF-20 amino acid sequence is fused at the amino-terminus of an FGF-20 or a fragment thereof. In another embodiment, such a chimeric protein is produced by recombinant expression of a nucleic acid encoding the protein (comprising an FGF-20-coding sequence joined in-frame to a non-FGF-20 coding sequence). Such a chimeric product can be custom made by a variety of companies (e.g., Retrogen, Operon, etc.) or made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, a chimeric nucleic acid encoding FGF-20 with a heterologous signal sequence is expressed such that the chimeric protein is expressed and processed by the cell to the mature FGF-20 protein. The primary sequence of FGF-20 and non-FGF-20 gene may also be used to predict tertiary structure of the molecules using computer simulation (Hopp and Woods, 1981, Proc. Natl. Acad. Sci. U.S.A. 78: 3824-3828); the chimeric recombinant genes could be designed in light of correlations between tertiary structure and biological function. Likewise, chimeric genes comprising an essential portion of FGF-20 molecule fused to a heterologous (non-FGF-20) protein-encoding sequence may be constructed. In a specific embodiment, such chimeric construction can be used to enhance one or more desired properties of an FGF-20, including but not limited to, FGF-20 stability, solubility, or resistance to proteases. In another embodiment, chimeric construction can be used to target FGF-20 to a specific site. In yet another embodiment, chimeric construction can be used to identify or purify an FGF-20 of the invention, such as a His-tag, a FLAG tag, a green fluorescence protein (GFP), β-galactosidase, a maltose binding protein (MalE), a cellulose binding protein (CenA) or a mannose protein, etc. In one embodiment, a CG53135 protein is carbamylated.

In some embodiment, a CG53135 protein can be modified so that it has an extended half-life in vivo using any methods known in the art. For example, inert polymer molecules such as high molecular weight polyethyleneglycol (PEG) can be attached to a CG53135 protein with or without a multifunctional linker either through site-specific conjugation of the PEG to the N- or C-terminus of the protein or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the CG53135 protein. Unreacted PEG can be separated from CG53135-PEG conjugates by size-exclusion or by ion-exchange chromatography. PEG-derivatized conjugates can be tested for in vivo efficacy using methods known to those of skill in the art.

A CG53135 protein can also be conjugated to albumin in order to make the protein more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

In some embodiments, CG53135 refers to CG53135-01 (SEQ ID NOs:1 and 2), CG53135-02 (SEQ ID NOs:3 and 4), CG53135-03 (SEQ ID NOs:5 and 2), CG53135-04 (SEQ ID NOs:6 and 7), CG53135-05 (SEQ ID NOs:8 and 2), CG53135-06 (SEQ ID NOs:9 and 10), CG53135-07 (SEQ ID NOs:11 and 12), CG53135-08 (SEQ ID NOs:13 and 14), CG53135-09 (SEQ ID NOs:15 and 16), CG53135-10 (SEQ ID NOs:17 and 18), CG53135-11 (SEQ ID NOs:19 and 20), CG53135-12 (SEQ ID NOs:21 and 22), CG53135-13 (SEQ ID NOs:23 and 24), CG53135-14 (SEQ ID NOs:25 and 26), CG53135-15 (SEQ ID NOs:27 and 28), CG53135-16 (SEQ ID NOs:29 and 30), CG53135-17 (SEQ ID NOs:31 and 32), IFC 250059629 (SEQ ID NOs:33 and 34), IFC 20059669 (SEQ ID NOs:35 and 36), IFC 317459553 (SEQ ID NOs:37 and 38), IFC 317459571 (SEQ ID NOs:39 and 40), IFC 250059596 (SEQ ID NOs:41 and 10), IFC316351224 (SEQ ID NOs:41 and 10), or a combination thereof. In a specific embodiment, a CG53135 is carbamylated, for example, a carbamylated CG53135-13 protein or a carbamylated CG53135-05 protein.

5.2 Methods of Preparing CG53135

Any techniques known in the art can be used in purifying a CG53135 protein, including but are not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See e.g., Scopes, PROTEIN PURIFICATION, PRINCIPLES AND PRACTICE, 3rd ed., Springer (1994). During the purification, the biological activity of CG53135 may be monitored by one or more in vitro or in vivo assays as described in Section 5.3, infra. The purity of CG53135 can be assayed by any methods known in the art, such as but not limited to, gel electrophoresis. See Scopes, supra. In some embodiment, the CG53135 proteins employed in a composition of the invention can be in the range of 80 to 100 percent of the total mg protein, or at least 80%, at least 85%, at least 90%, at least 95%, or at least 98% of the total mg protein. In one embodiment, one or more CG53135 proteins employed in a composition of the invention is at least 99% of the total protein. In another embodiment, CG53135 is purified to apparent homogeneity, as assayed, e.g., by sodium dodecyl sulfate polyacrylamide gel electrophoresis.

Methods known in the art can be utilized to recombinantly produce CG53135 proteins. A nucleic acid sequence encoding a CG53135 protein can be inserted into an expression vector for propagation and expression in host cells.

An expression construct, as used herein, refers to a nucleic acid sequence encoding a CG53135 protein operably associated with one or more regulatory regions that enable expression of a CG53135 protein in an appropriate host cell. “Operably-associated” refers to an association in which the regulatory regions and the CG53135 sequence to be expressed are joined and positioned in such a way as to permit transcription, and ultimately, translation.

The regulatory regions that are necessary for transcription of CG53135 can be provided by the expression vector. A translation initiation codon (ATG) may also be provided if a CG53135 gene sequence lacking its cognate initiation codon is to be expressed. In a compatible host-construct system, cellular transcriptional factors, such as RNA polymerase, will bind to the regulatory regions on the expression construct to effect transcription of the modified CG53135 sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to a CG53135 gene sequence or to insert a CG53135 gene sequence into the cloning site of a vector, linkers or adapters providing the appropriate compatible restriction sites may be ligated to the ends of the cDNAs by techniques well known in the art (see e.g., Wu et al., 1987, Methods in Enzymol, 152: 343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA using PCR with primers containing the desired restriction enzyme site.

An expression construct comprising a CG53135 sequence operably associated with regulatory regions can be directly introduced into appropriate host cells for expression and production of a CG53135 protein without further cloning. See, e.g., U.S. Pat. No. 5,580,859. The expression constructs can also contain DNA sequences that facilitate integration of a CG53135 sequence into the genome of the host cell, e.g., via homologous recombination. In this instance, it is not necessary to employ an expression vector comprising a replication origin suitable for appropriate host cells in order to propagate and express CG53135 in the host cells.

A variety of expression vectors may be used, including but are not limited to, plasmids, cosmids, phage, phagemids or modified viruses. Such host-expression systems represent vehicles by which the coding sequences of a CG53135 gene may be produced and subsequently purified, but also represent cells which may, when transformed or transfected with the appropriate nucleotide coding sequences, express CG53135 in situ. These include, but are not limited to, microorganisms such as bacteria (e.g., E. coli and B. subtilis) transformed with recombinant bacteriophage DNA, plasmid DNA or cosmid DNA expression vectors containing CG53135 coding sequences; yeast (e.g., Saccharomyces, Pichia) transformed with recombinant yeast expression vectors containing CG53135 coding sequences; insect cell systems infected with recombinant virus expression vectors (e.g., baculovirus) containing CG53135 coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing CG53135 coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli and eukaryotic cells are used for the expression of a recombinant CG53135 molecule. For example, mammalian cells such as Chinese hamster ovary cells (CHO) can be used with a vector bearing promoter element from major intermediate early gene of cytomegalovirus for effective expression of a CG53135 sequence (Foecking et al., 1986, Gene 45: 101; and Cockett et al., 1990, Bio/Technology 8: 2).

In bacterial systems, a number of expression vectors may be advantageously selected depending upon the use intended for the CG53135 molecule being expressed. For example, when a large quantity of a CG53135 is to be produced, for the generation of pharmaceutical compositions of a CG53135 molecule, vectors that direct the expression of high levels of fusion protein products that are readily purified may be desirable. Such vectors include, but are not limited to, the E. coli expression vector pCR2.1 TOPO (Invitrogen); pIN vectors (Inouye & Inouye, 1985, Nucleic Acids Res. 13: 3101-3109; Van Heeke & Schuster, 1989, J. Biol. Chem. 24: 5503-5509) and the like. Series of vectors like pFLAG (Sigma), pMAL (NEB), and pET (Novagen) may also be used to express the foreign proteins as fusion proteins with FLAG peptide, malE-, or CBD-protein. These recombinant proteins may be directed into periplasmic space for correct folding and maturation. The fused part can be used for affinity purification of the expressed protein. Presence of cleavage sites for specific protease like enterokinase allows to cleave off the CG53135 protein. The pGEX vectors may also be used to express foreign proteins as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. A CG53135 coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, a CG53135 coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing CG53135 in infected hosts (see, e.g., Logan & Shenk, 1984, Proc. Natl. Acad. Sci. USA 8 1: 355-359). Specific initiation signals may also be required for efficient translation of inserted CG53135 coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153: 51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, HeLa, COS, MDCK, 293, 3T3, W138, BT483, Hs578T, HTB2, BT2O and T47D, NSO (a murine myeloma cell line that does not endogenously produce any immunoglobulin chains), CRL7O3O and HsS78Bst cells. Expression in a bacterial or yeast system can be used if post-translational modifications are found to be non-essential for a desired activity of CG53135. In a preferred embodiment, E. coli is used to express a CG53135 sequence.

For long-term, high-yield production of properly processed CG53135, stable expression in cells is preferred. Cell lines that stably express CG53135 may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while CG53135 is expressed continuously.

A number of selection systems may be used, including but not limited to, antibiotic resistance (markers like Neo, which confers resistance to geneticine, or G-418 (Wu and Wu, 1991, Biotherapy 3: 87-95; Tolstoshev, 1993, Ann. Rev. Pharmacol. Toxicol. 32: 573-596; Mulligan, 1993, Science 260: 926-932; and Morgan and Anderson, 1993, Ann. Rev. Biochem. 62: 191-217; May, 1993, TIB TECH 11 (5): I55-2 15); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77:357; O'Hare et al., 1981, Proc. Natl. Acad. Sci. USA 78: 1527); gpt, which confers resistance to mycophenolic acid (Mulligan & Berg, 1981, Proc. Natl. Acad. Sci. USA 78: 2072); and hygro, which confers resistance to hygromycin (Santerre et al., 1984, Gene 30:147). In addition, mutant cell lines including, but not limited to, tk−, hgprt− or aprt− cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of CG53135. Modified culture conditions and media may also be used to enhance production of CG53135. Any techniques known in the art may be applied to establish the optimal conditions for producing CG53135.

An alternative to producing CG53135 or a fragment thereof by recombinant techniques is peptide synthesis. For example, an entire CG53135, or a protein corresponding to a portion of CG53135, can be synthesized by use of a peptide synthesizer. Conventional peptide synthesis or other synthetic protocols well known in the art may be used.

Proteins having the amino acid sequence of CG53135 or a portion thereof may be synthesized by solid-phase peptide synthesis using procedures similar to those described by Merrifield, 1963, J. Am. Chem. Soc., 85:2149. During synthesis, N-a-protected amino acids having protected side chains are added stepwise to a growing polypeptide chain linked by its C-terminal and to an insoluble polymeric support, i.e., polystyrene beads. The proteins are synthesized by linking an amino group of an N-a-deprotected amino acid to an a-carboxyl group of an N-a-protected amino acid that has been activated by reacting it with a reagent such as dicyclohexylcarbodiimide. The attachment of a free amino group to the activated carboxyl leads to peptide bond formation. The most commonly used N-a-protecting groups include Boc, which is acid labile and Fmoc which is base labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CG53135 protein is accomplished using conventional procedures, such as preparative HPLC using gel permeation, partition and/or ion exchange chromatography. The choice of appropriate matrices and buffers are well known in the art and so are not described in detail herein.

Non-limiting examples of methods for preparing CG53135 can be found in Section 6, infra.

5.3 Characterization and Demonstration of CG53135 Activities and Monitoring Effects During Treatment

Any methods known in the art can be used to determine the identity of a purified CG53135 protein in a composition used in accordance to the methods of the instant invention. Such methods include, but are not limited to, Western Blot, sequencing (e.g., Edman sequencing), liquid chromatography (e.g., HPLC, RP-HPLC with both UV and electrospray mass spectrometric detection), mass spectrometry, total amino acid analysis, peptide mapping, and SDS-PAGE. The secondary, tertiary and/or quaternary structure of a CG53135 protein can analyzed by any methods known in the art, e.g., far UV circular dichroism spectrum can be used to analyze the secondary structure, near UV circular dichroism spectroscopy and second derivative UV absorbance spectroscopy can be used to analyze the tertiary structure, and light scattering SEC-HPLC can be used to analyze quaternary structure.

The purity of a CG53135 protein in a composition used in accordance to the instant invention can be analyzed by any methods known in the art, such as but not limited to, sodium dodecyl sulphate polyacrylamide gel electrophoresis (“SDS-PAGE”), reversed phase high-performance liquid chromatography (“RP-HPLC”), size exclusion high-performance liquid chromatography (“SEC-HPLC”), and Western Blot (e.g., host cell protein Western Blot). In a preferred embodiment, a CG53135 protein in a composition used in accordance to the methods of the instant invention is at least 97%, at least 98%, or at least 99% pure by densitometry. In another preferred embodiment, a CG53135 protein in a composition used in accordance to the instant invention is more than 97%, more than 98%, or more than 99% pure by densitometry.

The biological activities and/or potency of CG53135 used in accordance with the present invention can be determined by any methods known in the art. For example, compositions for use in therapy in accordance to the methods of the present invention can be tested in suitable cell lines for one or more activities that FGF-20 possesses (e.g., cellular proliferation stimulatory activity). Non-limiting examples of such assays are described in Sections 6.4-6.10, infra.

Compositions for use in a therapy in accordance to the methods of the present invention can also be tested in suitable animal model systems prior to testing in humans. Such animal model systems include, but are not limited to, IBD models in rats, mice, hamsters, chicken, cows, monkeys, rabbits, etc. A non-limiting example of such animal model is mouse dosed with the sodium form of dextran sulfate (see, e.g., Section 6.11, infra).

To establish an estimate of drug activity in IBD model experiments, an index can be developed that combines observational examination of the animals as well as their survival status. Non-limiting examples are given in Section 6, infra. Any staging/scoring system for human patients known in the art may also be used.

Further, any assays known to those skilled in the art can be used to evaluate the prophylactic and/or therapeutic utilities of the combinatorial therapies disclosed herein for prevention and/or treatment of inflammatory bowel disease.

The effectiveness of CG53135 on preventing and/or treating inflammatory bowel disease can be monitored by any methods known to one skilled in the art, including but not limited to, clinical evaluation, and measuring the level of CG53135 biomarkers in a biosample.

Any adverse effects during the use of CG53135 alone or in combination with another therapy (e.g., another therapeutic or prophylactic agent) are preferably also monitored. Examples of adverse effects of chemotherapies used in managing IBD can be found, e.g., in the Physicians' Desk Reference (58th ed., 2004).

5.4 Prophylactic and Therapeutic Uses

In one embodiment, the present invention provides methods of preventing and/or treating a pathology of epithelial cells and/or mesenchymal cells comprising administering to a subject in need thereof a composition comprising one or more CG53135 proteins. In another embodiment, the present invention provides methods of stimulating proliferation, differentiation or migration of epithelial cells and/or mesenchymal cells comprising administering to a subject in need thereof an effective amount of a composition comprising one or more CG53135 proteins.

Epithelial membranes are continuous sheets of cells with contiguous cell borders that have characteristic specialized sites of close contact called cell junction. Such membrane, which can be one or more cells thick, contain no capillaries. Epithelia are attached to the underlying connective tissue by a component known as a basement membrane, which is a layer of intercellular material of complex composition that is distributed as a thin layer between the epithelium and the connective tissue.

Stratified squamous nonkeratinizing epithelium is common on wet surfaces that are subject to considerable wear and tear at sites where absorptive function is not required. The secretions necessary to keep such surfaces wet have to come from appropriately situated glands. Sites lined by this type of epithelium include the esophagus and the floor and sides of the oral cavity.

Simple columnar epithelium is made up of a single layer of tall cells that again fit together in a hexagonal pattern. In simple secretory columnar epithelium, the columnar cells are all specialized to secret mucus in addition to being protective. Sites of this type of epithelium is present include the lining of the stomach.

A simple columnar epithelium that is made up of absorptive cells as well as secretory cells lines the intestine. To facilitate absorption, this membrane is only one cell thick. Interspersed with cells that are specialized for absorption, there are many goblet cells that secrete protective mucus.

Mesenchymal cells are stem cells that can differentiate into, e.g., osteoblasts, chondrocytes, myocytes, and adipocytes. Mesenchymal-epithelial interactions play an important role in the physiology and pathology of epithelial tissues. Mesenchymal cells may associate with epithelium basement membrane (e.g., pericytes and perivascular monocyte-derived cells (MDCs)), or reside within epithelium (MDCs and T cells). The nature of the interactions between mesenchymal cells and tissue-specific cells may depend on the tissue type (e.g., brain versus epidermis), or on the prevention or allowance/stimulation of differentiation of cells into the suicidal state (apoptosis) by mesenchymal cells in a given epithelium. Specialized mesenchymal cells, such as pericytes, MDCs, and T lymphocytes, may significantly influence the differentiation and aging of epithelial cells.

The stromal compartment of the cavities of bone is composed of a net-like structure of interconnected mesenchymal cells. Stromal cells are closely associated with bone cortex, bone trabecule and to the hemopoietic cells. The bone marrow-stromal micro-environment, is a complex of cells, extracellular matrix (ECM) with growth factors and cytokines that regulate osteogenesis and hemopoiesis locally throughout the life of the individual. The role of the marrow stroma in creating the microenvironment for bone physiology and hemopoiesis lies in a specific subpopulation of the stroma cells. They differentiate from a common stem cell to the specific lineage each of which has a different role. Their combined function results in orchestration of a 3-D-architecture that maintains the active bone marrow within the bone.

In adults, blood cells are produced by the bone marrow, the spongy material filling the body's bones. The bone marrow produces two blood cell groups, myeloid and lymphoid. The myeloid cell line includes, e.g., the following: (1) Immature cells called erythrocytes that later develop into red blood cells; (2) Blood clotting agents (platelets); (3) Some white blood cells, including macrophages (which act as scavengers for foreign particles), eosinophils (which trigger allergies and also defend against parasites), and neutrophils (the main defenders against bacterial infections). The lymphoid cell line includes, e.g., the lymphocytes, which are the body's primary infection fighters. Among other vital functions, certain lymphocytes are responsible for producing antibodies, factors that can target and attack specific foreign agents (antigens). Lymphocytes develop in the thymus gland or bone marrow and are therefore categorized as either B-cells (bone marrow-derived cells) or T-cells (thymus gland-derived cells).

According to the present invention, a CG53135 protein can regulate proliferation, differentiation, and/or migration of epithelial cells and/or mesenchymal cells, and thus have prophylactic and/or therapeutic effects on a disorder associated with a pathology of epithelial cells and/or mesenchymal cells.

The present invention provides methods of preventing and/or treating inflammatory bowel disease or irritable bowel syndrome comprising administering to a subject in need thereof an effective amount of a composition comprising one or more isolated CG53135 proteins. Inflammatory bowel disease that can be prevented and/or treated by the methods of the invention includes, but is not limited to, ulcerative colitis and Crohn's disease.

The present invention provides methods of preventing and/or treating inflammatory bowel disease in patient populations with inflammatory bowel disease and populations at risk to develop inflammatory bowel disease. The present invention also provides methods of preventing and/or treating irritable bowel syndrome in patient populations with irritable bowel syndrome and populations at risk to develop irritable bowel syndrome.

In one embodiment, the present invention provides a method of preventing or treating inflammatory bowel disease or irritable bowel syndrome comprising administering to a subject in need thereof a prophylactically or therapeutically effective amount of an isolated protein selected from the group consisting of: (a) a protein comprising an amino acid sequence of SEQ ID NOs:2, 4, 7, 10, 22, 24, 26, 28, 30, 32, 34, 36, 38, or 40; (b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and (c) a fragment of the protein of (a) or (b), which fragment retains cell proliferation stimulatory activity.

In some embodiments, the present invention provides a method of preventing and/or treating inflammatory bowel disease or irritable bowel syndrome comprising cyclically administering a composition comprising one or more CG53135 proteins. In one embodiment, cycling therapy involves the administration of a first therapy for a period of time, followed by the administration of a second therapy for a period of time and repeating this sequential administration, i.e., the cycle, in order to, e.g., to avoid or reduce the side effects of one of the therapies and/or to improve the efficacy of the therapies. In another embodiment, cycling therapy involves the administration of a therapy for a period of time, stop the therapy for a period of time, and repeat the administration of the therapy.

In accordance to the instant invention, a composition comprising one or more isolated CG53135 proteins can also be used in combination with other therapies to prevent and/or treat inflammatory bowel disease or irritable bowel syndrome. In one embodiment, a composition comprising one or more isolated CG53135 proteins is administered in combination with one or more other therapies (e.g., therapeutic agents) that have prophylactic and/or therapeutic effect(s) on inflammatory bowel disease and/or have amelioration effect(s) on one or more symptoms associated with inflammatory bowel disease to a subject to prevent and/or treat inflammatory bowel disease. Non-limiting examples of such therapies are: 5-aminosalicylates, antibiotics, corticosteroids, immunomodultors (e.g., 6-mercaptoputine, azathioprine, methotrexate, cyclosporine), and biological response modifiers (e.g., infliximab). In another embodiment, a composition comprising one or more isolated CG53135. proteins is administered in combination with one or more other therapies (e.g., therapeutic agents) that have prophylactic and/or therapeutic effect(s) on irritable bowel syndrome and/or have amelioration effect(s) on one or more symptoms associated with irritable bowel syndrome to a subject to prevent and/or treat inflammatory bowel disease. Non-limiting examples of such therapies are: laxatives; antidiarrheals (e.g., diphenoxylate (e.g., Lomotil, Lomocot); loperamide (e.g., Imodium, Pepto Diarrhea), cholestyramine (e.g., Questran, Cholybar)); antispasmodics (e.g., dicyclomine, hyoscyamine, and clidinium (in combination with chlordiazepoxide hydrochloride)); peppermint oil; direct smooth muscle relaxants; antidepressants; 5-HT3 antagonists (e.g., Alosetron (Lotronex), cilansetron); 5-HT4 agonists (e.g., tegaserod (Zelnorm/Zelmac) and prucalopride); M3 receptor antagonists (e.g., zamifenacin and darifenacin).

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

In one embodiment, the dosage of a composition comprising one or more G53135 proteins for administration in a human patient provided by the present invention is at least 0.001 mg/kg, at least 0.005 mg/kg, at least 0.01 mg/kg, at least 0.03 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, or at least 10 mg/kg (as measured by UV assay). In another embodiment, the dosage of a composition comprising one or more CG53135 proteins for administration in a human patient provided by the present invention is between 0.001-10 mg/kg, between 0.005-5 mg/kg, between 0.01-1 mg/kg, between 0.01-0.9 mg/kg, between 0.01-0.8 mg/kg, between 0.01-0.7 mg/kg, between 0.01-0.6 mg/kg, between 0.01-0.5 mg/kg, or between 0.01-0.3 mg/kg (as measured by UV assay).

Protein concentration can be measured by methods known in the art, such as Bradford assay or UV assay, and the concentration may vary depending on what assay is being used. In a non-limiting example, the protein concentration in a pharmaceutical composition of the instant invention is measured by a UV assay that uses a direct measurement of the UV absorption at a wavelength of 280 nm, and calibration with a well characterized reference standard of CG53135 protein (instead of IgG). Test results obtained with this UV method (using CG53135 reference standard) are three times lower than test results for the same sample(s) tested with the Bradford method (using IgG as calibrator). For example, if a dosage of a composition comprising one or more CG53135 proteins for administration in a human patient provided by the present invention is between 0.001-10 mg/kg measured by UV assay, then the dosage is 0.003-30 mg/kg as measured by Bradford assay.

In one embodiment, prior to administering the first full dose, each patient preferably receives a subcutaneous injection of a small amount (e.g., 1/100 to 1/10 of the prescribed dose) of a composition of the invention to detect any acute intolerance. The injection site is examined one and two hours after the test. If no reaction is detected, then the full dose is administered.

5.5 Pharmaceutical Compositions

The compositions used in accordance to methods of the invention can be administered to a subject at a prophylactically or therapeutically effective amount to prevent and/or treat inflammatory bowel disease or irritable bowel syndrome. Various delivery systems are known and can be used to administer a composition used in accordance to the methods of the invention. Such delivery systems include, but are not limited to, encapsulation in liposomes, microparticles, microcapsules, expression by recombinant cells, receptor-mediated endocytosis, construction of the nucleic acids of the invention as part of a retroviral or other vectors, etc. Methods of introduction include, but are not limited to, intradermal, intramuscular, intraperitoneal, intrathecal, intracerebroventricular, epidural, intravenous, subcutaneous, intranasal, intratumoral, transdermal, transmucosal, rectal, and oral routes. The compositions used in accordance to the methods of the invention may be administered by any convenient route, for example, by infusion or bolus injection, by absorption through epithelial or mucocutaneous linings (e.g., eye mucosa, oral mucosa, vaginal mucosa, rectal and intestinal mucosa, etc.), and may be administered together with other biologically active agents. Administration can be systemic or local. In a specific embodiment, the present invention comprises using single or double chambered syringes, preferably equipped with a needle-safety device and a sharper needle, that are pre-filled with a composition comprising one or more CG53135 proteins. In one embodiment, dual chambered syringes (e.g., Vetter Lyo-Ject dual-chambered syringe by Vetter Pharmar-Fertigung) are used. Such systems are desirable for lyophilized formulations, and are especially useful in an emergency setting.

In some embodiments, it may be desirable to administer the pharmaceutical compositions of the invention locally to the area in need of treatment. This may be achieved by, for example, local infusion during surgery, or topical application, e.g., in conjunction with a wound dressing after surgery, by injection, by means of a catheter, by means of a suppository, or by means of an implant (said implant being of a porous, non-porous, or gelatinous material, including membranes, such as sialastic membranes, or fibers). In one embodiment, administration can be by direct injection at the site (or former site) of rapidly proliferating tissues that are most sensitive to an insult, such as radiation, chemotherapy, or chemical/biological warfare agent.

In some embodiments, where the composition of the invention is a nucleic acid encoding a prophylactic or therapeutic agent, the nucleic acid can be administered in vivo to promote expression of their encoded proteins (e.g., CG53135 proteins), by constructing the nucleic acid as part of an appropriate nucleic acid expression vector and administering it so that it becomes intracellular, e.g., by use of a retroviral vector, or by direct injection, or by use of microparticle bombardment (e.g., a gene gun), or coating with lipids or cell-surface receptors or transfecting agents, or by administering it in linkage to a homeobox-like peptide which is known to enter the nucleus, etc. Alternatively, a nucleic acid of the invention can be introduced intracellularly and incorporated within host cell DNA for expression, by homologous recombination.

The instant invention encompasses bulk drug compositions useful in the manufacture of pharmaceutical compositions that can be used in the preparation of unit dosage forms. In a preferred embodiment, a composition of the invention is a pharmaceutical composition. Such compositions comprise a prophylactically or therapeutically effective amount of CG53135, and a pharmaceutically acceptable carrier. Preferably, the pharmaceutical compositions are formulated to be suitable for the route of administration to a subject.

In one embodiment, the term “pharmaceutically acceptable” means approved by a regulatory agency of the Federal or a state government or listed in the U.S. Pharmacopeia or other generally regarded as safe for use in humans (GRAS). The term “carrier” refers to a diluent, adjuvant, bulking agent (e.g., arginine in various salt forms, sulfobutyl ether Beta-cyclodextrin sodium, or sucrose), excipient, or vehicle with which CG53135 is administered. Such pharmaceutical carriers can be sterile liquids, such as water and oils (e.g., oils of petroleum, animal, vegetable or synthetic origins, such as peanut oil, soybean oil, mineral oil, sesame oil and the like), or solid carriers, such as one or more substances which may also act as diluents, flavoring agents, solubilizers, lubricants, suspending agents, or encapsulating material. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch or its synthetically modified derivatives such as hydroxyethyl starch, stearate salts, glycerol, glucose, lactose, sucrose, trehalose, gelatin, sulfobutyl ether Beta-cyclodextrin sodium, sodium chloride, glycerol, propylene, glycol, water, ethanol, or a combination thereof. The composition, if desired, can also contain minor amounts of wetting or emulsifying agents, or pH buffering agents.

The compositions comprising CG53135 may be formulated into any of many possible dosage forms such as, but not limited to, liquid, suspension, microemulsion, microcapsules, tablets, capsules, gel capsules, soft gels, pills, powders, enemas, sustained-release formulations and the like. The compositions comprising CG53135 may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. The composition can also be formulated as a suppository, with traditional binders and carriers such as triglycerides. Oral formulation can include standard carriers, such as pharmaceutical grades of mannitol, lactose, starch or its synthetically modified derivatives such as hydroxyethyl starch, stearate salts, sodium saccharine, cellulose, magnesium carbonate, etc.

A pharmaceutical composition comprising CG53135 is formulated to be compatible with its intended route of administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic or hypertonic aqueous buffer. Where necessary, the composition may also include a solubilizing agent and a local anesthetic such as benzyl alcohol or lidocaine to ease pain at the site of the injection.

If a composition comprising CG53135 is to be administered topically, the composition can be formulated in the form of transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compositions of the invention are in admixture with a topical delivery agent, such as but not limited to, lipids, liposomes, fatty acids, fatty acid esters, steroids, chelating agents and surfactants. The compositions comprising CG53135 may be encapsulated within liposomes or may form complexes thereto, in particular to cationic liposomes. Alternatively, the compositions comprising CG53135 may be complexed to lipids, in particular to cationic lipids. For non-sprayable topical dosage forms, viscous to semi-solid or solid forms comprising a carrier or one or more excipients compatible with topical application and having a dynamic viscosity preferably greater than water are typically employed. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon or hydrofluorocarbons) or in a squeeze bottle. Moisturizers or humectants can also be added to pharmaceutical compositions and dosage forms if desired. Examples of such additional ingredients are well-known in the art.

A composition comprising CG53135 can be formulated in an aerosol form, spray, mist or in the form of drops or powder if intranasal administration is preferred. In particular, a composition comprising CG53135 can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, other hydrofluorocarbons, carbon dioxide or other suitable gas). In the case of a pressurized aerosol the dosage unit may be determined by providing a valve to deliver a metered amount. Microcapsules (composed of, e.g., polymerized surface) for use in an inhaler or insufflator may be formulated containing a powder mix of the compound and a suitable powder base such as dissacharides or starch.

One or more CG53135 proteins may also be formulated into a microcapsule with one or more polymers (e.g., hydroxyethyl starch) form the surface of the microcapsule. Such formulations have benefits such as slow-release.

A composition comprising CG53135 can be formulated in the form of powders, granules, microparticulates, nanoparticulates, suspensions or solutions in water or non-aqueous media, capsules, gel capsules, sachets, tablets or minitablets if oral administration is preferred. Thickeners, flavoring agents, diluents, emulsifiers, dispersing aids or binders may be desirable. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

In one embodiment, the compositions of the invention are orally administered in conjunction with one or more penetration enhancers, e.g., alcohols, surfactants and chelators. Preferred surfactants include, but are not limited to, fatty acids and esters or salts thereof, bile acids and salts thereof. In some embodiments, combinations of penetration enhancers are used, e.g., alcohols, fatty acids/salts in combination with bile acids/salts. In a specific embodiment, sodium salt of lauric acid, capric acid is used in combination with UDCA. Further penetration enhancers include, but are not limited to, polyoxyethylene-9-lauryl ether, polyoxyethylene-20-cetyl ether. Compositions of the invention may be delivered orally in granular form including, but is not limited to, sprayed dried particles, or complexed to form micro or nanoparticles. Complexing agents that can be used for complexing with the compositions of the invention include, but are not limited to, poly-amino acids, polyimines, polyacrylates, polyalkylacrylates, polyoxethanes, polyalkylcyanoacrylates, cationized gelatins, albumins, acrylates, polyethyleneglycols (PEG), DEAE-derivatized polyimines, pollulans, celluloses, and starches. Particularly preferred complexing agents include, but are not limited to, chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

A composition comprising CG53135 can be delivered to a subject by pulmonary administration, e.g., by use of an inhaler or nebulizer, of a composition formulated with an aerosolizing agent.

In a preferred embodiment, a composition comprising CG53135 is formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as benzyl alcohol or lidocaine to ease pain at the site of the injection. Generally, the ingredients are supplied either separately or mixed together in unit dosage form, for example, as a dry lyophilized powder or water free concentrate in a sealed container, such as a vial, ampoule or sachette, indicating the quantity of active agent. Where the composition is to be administered by infusion, it can be dispensed with an infusion container containing sterile pharmaceutical grade water or saline. Where the composition is administered by injection, an ampoule or vial of sterile water for injection or saline can be provided so that the ingredients may be mixed prior to administration.

A composition comprising CG53135 can be formulated as neutral or salt forms. Pharmaceutically acceptable salts include, but are not limited to, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In addition to the formulations described previously, a composition comprising CG53135 may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

In one embodiment, the ingredients of the compositions used in accordance to the methods of the invention are derived from a subject that is the same species origin or species reactivity as recipient of such compositions.

In some embodiments, a formulation used in accordance to the methods of the invention comprises 0.02 M-0.2 M acetate, 0.5-5% glycerol, 0.2-0.5 M arginine-HCl, and one ore more CG53135 proteins, preferably 0.5-5 mg/ml (UV). In one embodiment, a formulation used in accordance to the methods of the invention comprises 0.04M sodium acetate, 3% glycerol (volume/volume), 0.2 M arginine-HCl at pH 5.3, and one or more isolated CG53135 proteins, preferably 0.8 mg/ml (UV). In some embodiments, a formulation used in accordance to the methods of the invention comprises 0.01-1 M of a stabilizer, such as arginine in various salt forms, sulfobutyl ether Beta-cyclodextrin sodium, or sucrose, 0.01-0.1 M sodium phosphate monobasic (NaH₂PO₄.H₂O), 0.01%-0.1% weight/volume (“w/v”) polysorbate 80 or polysorbate 20, and one or more CG53135 proteins, preferably 0.005-50 mg/ml (UV). In one embodiment, a formulation used in accordance to the methods of the invention comprises 30 mM sodium citrate, pH 6.1, 2 mM EDTA, 200 mM sorbitol, 50 mM KCl, 20% glycerol, and one or more isolated CG53135 proteins.

The invention also provides kits for carrying out the therapeutic regimens of the invention. Such kits comprise in one or more containers prophylactically or therapeutically effective amounts of the composition of the invention (e.g., a composition comprising one or more CG53135 proteins) in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe, preferably packaged in sterile form, for injecting the formulation, and/or a packaged alcohol pad. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention (e.g., a composition comprising one or more CG53135 proteins) sufficient for a single administration.

As with any pharmaceutical product, the packaging material and container are designed to protect the stability of the product during storage and shipment. In one embodiment, compositions of the invention are stored in containers with biocompatible detergents, including but not limited to, lecithin, taurocholic acid, and cholesterol; or with other proteins, including but not limited to, gamma globulins and serum albumins. Further, the products of the invention include instructions for use or other informational material that advise the physician, technician, or patient on how to appropriately prevent or treat the disease or disorder in question.

### 6. EXAMPLE

Certain embodiments of the invention are illustrated by the following non-limiting examples.

### 6.1 Example 1

### Identification of Single Nucleotide Polymorphisms in FGF-20 Nucleic Acid Sequences

This example demonstrated how some of the single nucleotide polymorphisms (SNPs) of FGF-20 were identified. A SNP can, in some instances, be referred to as a “cSNP” to denote that the nucleotide sequence containing the SNP originates as a cDNA. SNPs occurring within a gene may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Non-limiting examples include alteration in temporal expression, physiological response regulation, cell type expression regulation, intensity of expression, and stability of transcribed message.

SeqCalling™ assemblies produced by the exon linking process were selected and extended using the following criteria: genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling™ assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling™ assemblies map to those regions. Such SeqCalling™ sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling™ database. SeqCalling™ fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling™ assemblies and genomic clones was reiterated to derive the full length sequence (Alderborn et al., Genome Research 10 (8) 1249-1265 (2000)).

Variants are reported individually in Table 2, but any combination of all or select subset of the variants is also encompassed by the present invention.

TABLE 2
SNPs of CG53135-01 (SEQ ID NOs: 1 and 2)

| | Nucleotides | | | Amino Acids | | |
|---|---|---|---|---|---|---|
| Variant | Position | Initial | Modified | Position | Initial | Modified |
| 13377871 | 301 | A | G | 101 | Ile | Val |
| 13375519 | 361 | A | G | 121 | Met | Val |
| 13375518 | 517 | G | A | 173 | Gly | Arg |
| 13375516 | 523 | C | G | 175 | Pro | Ala |
| 13381791 | 616 | G | A | 206 | Asp | Asn |

### 6.2 Example 2

### EXPRESSION OF CG53135

Several different expression constructs were generated to express CG53135 proteins (Table 3). The CG53135-05 construct, a codon-optimized, phage-free construct encoding the full-length gene (construct #3 in Table 3), was expressed in E. coli BLR (DE3), and the purified protein product was used in toxicology studies and clinical trials.

TABL3 3
Constructs Generated to Express CG53135

| Construct | Construct Description | Construct Diagram |
|---|---|---|
| 1a | NIH 3T3 cells were transfected with pFGF-20, which incorporates an epitope tag (V5) and a polyhistidine tag into the carboxy-terminus of the CG53135-01 protein in the pcDNA3.1 vector (Invitrogen) | 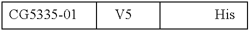 |
| 1b | Human 293-EBNA embryonic kidney cells or NIH 3T3 cells were transfected with CG53135-01 using pCEP4 vector (Invitrogen) containing an IgK signal sequence, multiple cloning sites, a V5 epitope tag, and a polyhistidine tag | 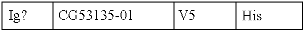 |
| 2 | E. coli BL21 cells were transformed with CG53135-01 using pETMY vector (CuraGen Corporation) containing a polyhistidine tag and a T7 epitope tag (this construct is also referred to as E. colilpRSET) | 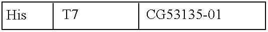 |
| 3 | E. coil BLR (DE3) cells (NovaGen) were transformed with CG53135-05 (full-length, codon-optimized) using pET24a vector (NovaGen) | 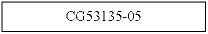 |
| 4 | E. coil BLR (DE3) cells (NovaGen) were transformed with CG53135 (deletion of amino acids 2-54, codon-optimized) using pET24a vector (NovaGen) | 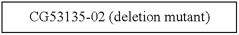 |

In one construct, CG53135-01 (the full-length CG53135 gene) was cloned as a Bgl II-Xho I fragment into the Bam HI-Xho I sites in mammalian expression vector, pcDNA3.1V5His (Invitrogen Corporation, Carlsbad, Calif.). The resultant construct, pFGF-20 (construct 1a) has a 9 amino acid V5 tag and a 6 amino acid histidine tag (His) fused in-frame to the carboxy-terminus of CG53135-01. These tags aid in the purification and detection of CG53135-01 protein. After transfection of pFGF-20 into murine NIH 3T3 cells, CG53135-01 protein was detected in the conditioned medium using an anti-V5 antibody (Invitrogen, Carlsbad, Calif.).

The full-length CG53135-01 gene was also cloned as a Bgl II-Xho I fragment into the Bam HI-Xho I sites of mammalian expression vector pCEP4/Sec (CuraGen Corporation). The resultant construct, plg?-FGF-20 (construct 1b) has a heterologous immunoglobulin kappa (IgK) signal sequence that could aid in secretion of CG53135-01. After transfection of plgK-FGF-20 into human 293 EBNA cells (Invitrogen, Carlsbad, Calif.; catalog # R620-07), CG53135-01 was detected in the conditioned medium using an anti-V5 antibody.

In order to increase the yield of CG53135 protein, a Bgl II-Xho I fragment encoding the full-length CG53135-01 gene was cloned into the Bam HI-Xho I sites of E. coli expression vector, pETMY (CuraGen Corporation). The resultant construct, pETMY-FGF-20 (construct 2) has a 6 amino acid histidine tag and a T7 tag fused in-frame to the amino terminus of CG53135. After transformation of pETMY-FGF-20 into BL21 E. coli (Novagen, Madison, Wis.), followed by T7 RNA polymerase induction, CG53135-01 protein was detected in the soluble fraction of the cells.

In order to express CG53135 without tags, CG53135-05 (a codon-optimized, full-length FGF-20 gene) and CG53135-02 (a codon-optimized deletion construct of FGF-20, with the N-terminal amino acids 2-54 removed) were synthesized. For the full-length construct (CG53135-05), an Nde I restriction site (CATATG) containing the initiator codon was placed at the 5′ end of the coding sequence. At the 3′ end, the coding sequence was followed by 2 consecutive stop codons (TM) and a Xho restriction site (CTCGAG). The synthesized gene was cloned into pCRScript (Stratagene, La Jolla, Calif.) to generate pCRScript-CG53135. An Nde I-Xho I fragment containing the codon-optimized CG53135 gene was isolated from the pCRscript-CG53135 and subcloned into Nde I-Xho I-digested pET24a to generate pET24a-CG53135 (construct 3). The full-length, codon-optimized version of CG53135 is referred to as CG53135-05.

To generate a codon-optimized deletion construct for CG53135, oligonucleotide primers were designed to amplify the deleted CG53135 gene from pCRScript-CG53135. The forward primer contained an Nde I site (CATATG) followed by coding sequence starting at amino acid 55. The reverse primer contained a HindIII restriction site. A single PCR product of approximately 480 base pairs was obtained and cloned into pCR2.1 vector (Invitrogen) to generate pCR2.1-CG53135del. An Nde 1-Hind III fragment was isolated from pCR2.1-53135del and subcloned into Nde 1-Hind III-digested pET24a to generate pET24a-CG53135-02 (construct 4).

The plasmids, pET24a-CG53135-05 (construct 3) and pET24a-CG53135-02 (construct 4) have no tags. Each vector was transformed into E. coli BLR (DE3), induced with isopropyl thiogalactopyranoside. Both the full-length and the N-terminally truncated CG53135 protein was detected in the soluble fraction of cells.

### 6.3 Example 3

### Proteolytic Cleavage Products of CG53135-05

When pET24a-CG53135-05 (construct 3, see Example 2) was expressed in E. coli (DE3) and the protein was purified according to Process 1 as described in Section 6.18.1 and Process 2 as described in Section 6.18.2, respectively, the final purified protein product from each process was analyzed using techniques such as Liquid Chromatography, Mass spectrometry and N-terminal sequencing. Such analyses indicate that the final purified protein product includes some truncated form of FGF-20 (e.g., CG53135-13 (SEQ ID NO:24), CG53135-15 (SEQ ID NO:28), CG53135-16 (SEQ ID NO:30), and CG53135-17 (SEQ ID NO:32)) in addition to the full length FGF-20, and a protein consisting of amino acids 3-211 (CG53135-13, SEQ ID NO:24) of FGF-20 constitutes the majority of the final purified protein product.

All the variants/fragments in the final purified product have high activity in the proliferation assays. Thus these variants/fragments are expected to have same utility as that of FGF-20. For the purpose of convenience, the term “CG53135-05 E. coli purified product” is used herein to refer to a purified protein product from E. coli expressing a CG53135-05 construct. For example, a CG53135-05 E. coli purified product may contain a mixture of the full length CG53135-05 protein (SEQ ID NO:2), CG53135-13 (SEQ ID NO:24), CG53135-15 (SEQ ID NO:28), CG53135-16 (SEQ ID NO:30), and CG53135-17 (SEQ ID NO:32), with the majority of the content being CG53135-13 (SEQ ID NO:24).

RP-HPLC Assay: Peak Identification

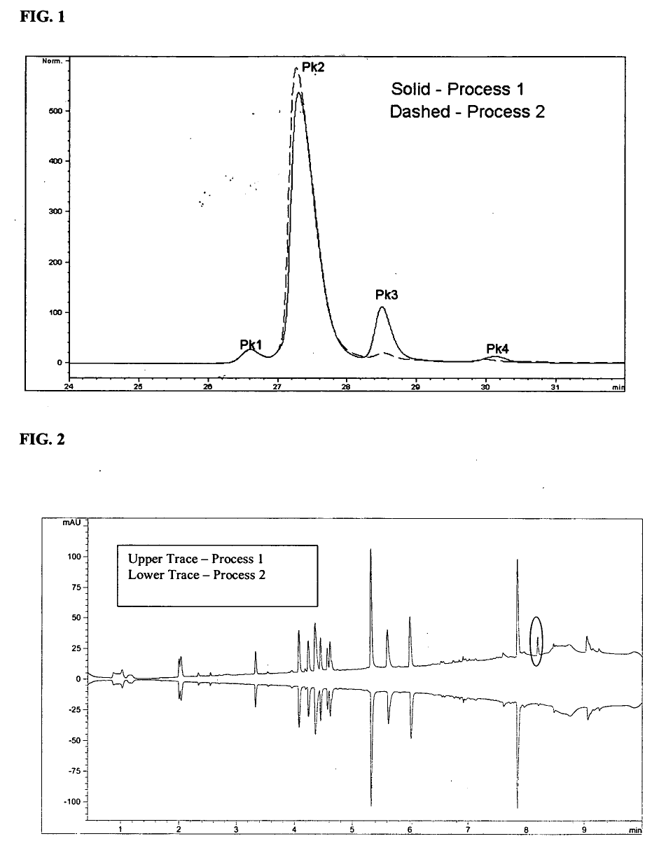

Purified drug substance (by both Process 1 and Process 2, respectively) was further analyzed by reversed-phase high-performance liquid chromatography (RP-HPLC) with both UV and electrospray mass spectrometric detection. Purified protein from either Process 1 or Process 2 was loaded onto a Protein C4 column (Vydac, 5 μm, 150 mm×4.6 mm) using a standard HPLC system in a mobile phase containing water, acetonitrile and trifluoroacetic acid. The elution gradient for this method was modified to resolve four distinct chromatographic peaks eluting at 26.6, 27.3, 28.5 and 30.0 min respectively (FIG. 1). These peaks were characterized by electrospray mass spectrometry. As can be observed from the chromatograms, the four equipotent isoforms are present in the purified final product from Process 1 and 2. However, the proportion of these peaks (1, 3 and 4) is much lower in the final product purified by Process 2 with the predominant form being Peak 2.

The identities of each peak from the RP-HPLC separation are indicated in Table 4.

TABLE 4
Identity of peaks from the RP-HPLC separation of CG53135-05 E. coli purified
product based upon accurate molecular weight determination.

| | | Molecular | | | Predicted |
|---|---|---|---|---|---|
| | Retention | Weight | Assignment | | Molecular |
| Peak # | Time (min) | Observed | (residue #) | ID Number | Weight |
| 1 | 26.6 | 21329.2 | 24-211 | CG53135-17 | 21329.2 |
| 1 | 26.6 | 22185.1 | 15-211 | CG53135-16 | 22185.1 |
| 1 | 26.6 | 22412.4 | 12-211 | CG53135-15 | 22412.4 |
| 2 | 27.3 | 23296.5 | 3-211 | CG53135-13 | 23296.4 |
| 3 | 28.5 | 23498.9 | 1-211 | CG53135-05 | 23498.7 |
| 4 | 30.0 | 23339.3 | 3-211 | CG53135-13 | 23339.4 |
| | | | (carbamylated) | (carbamylated) | |
| 4 | 30.0 | 23539.7 | 1-211 | CG53135-05 | 23539.7 |
| | | | (carbamylated) | (carbamylated) | |

Edman Sequencing and Total Amino Acid Analysis

The experimental N-terminal amino acid sequence of the Process 1 reference standard, DEV10, and the Process 2 interim reference standard were determined qualitatively. The reference standards were resolved by SDS-PAGE and electrophoretically transferred to a polyvinylidenefluoride membrane; the Coomassie-stained ˜23 kDa major band corresponding to each reference standard was excited from the membrane and analyzed by an automated Edman sequencer (Procise, Applied Biosystems, Foster City, Calif.). A comparison of the two major sequences is shown in Table 5 below. The predominant sequence for each reference standard was identical and corresponded to residues 3-20 in the theoretical N-terminal sequence of CG53135-05.

TABLE 5
Edman sequencing data for the first 20 amino acids of CG53135-05
E. coli purified product for Process 1 and 2.

| | |
|---|---|
| Theoretical | |
| Residue | Amino Acid Residue |

| Position | Process 1 | Process 2 |
|---|---|---|
| 3 | Pro | Pro |
| 4 | Leu | Leu |
| 5 | Ala | Ala |
| 6 | Glu | Glu |
| 7 | Val | Val |
| 8 | Gly | Gly |
| 9 | Gly | Gly |
| 10 | Phe | Phe |
| 11 | Leu | Leu |
| 12 | Gly | Gly |
| 13 | Gly | Gly |
| 14 | Leu | Leu |
| 15 | Glu | Glu |
| 16 | Gly | Gly |
| 17 | Leu | Leu |
| 18 | Gly | Gly |
| 19 | Gln | Gln |
| 20 | Gln | Gln |

The experimental amino acid composition of the DEV10 reference standard and the PX3536G001-H reference standard were determined in parallel. Quadruplicate samples of each reference standard were hydrolyzed for 16 hours at 115° C. in 100 μL of 6 N HCl, 0.2% phenol containing 2 nmol norleucine as an internal standard. Samples were dried in a Speed Vac Concentrator and dissolved in 100 μL sample buffer containing 2 nmol homoserine as an internal standard. The amino acids in each sample were separated on a Beckman Model 7300 amino acid analyzer. The amino acid composition of both reference standards showed no significant differences as shown in Table 6 below. Note that Cys and trp are destroyed during acid hydrolysis of the protein. Asn and gln are converted to asp and glu, respectively, during acid hydrolysis and thus their respective totals are reported as asx and glx. Met and his were both unresolved in this procedure.

TABLE 6
Quantitive amino acid analysis for CG53135-05 E. coli purified
product from Process 1 and Process 2

| | |
|---|---|
| Amino Acid | Mole Percent |

| Residue | DEV10 | PX3536G001-H |
|---|---|---|
| asx | 7.1 | 7.0 |
| thr | 4.0 | 4.0 |
| ser | 6.3 | 6.1 |
| glx | 12.2 | 12.2 |
| pro | 6.0 | 6.0 |
| gly | 14.4 | 14.3 |
| ala | 5.8 | 5.6 |
| val | 5.3 | 5.3 |
| ile | 3.5 | 3.5 |
| leu | 13.6 | 13.6 |
| tyr | 4.6 | 4.6 |
| phe | 5.2 | 5.2 |
| lys | 3.7 | 3.7 |
| arg | 8.5 | 9.1 |

Tryptic Mapping by RP-HPLC

Purified drug substance from Process 1 and 2 was reduced and alklated with iodoacetic acid and then digested with sequencing grade trypsin. The tryptic peptides were separated by reversed-phase high-performance liquid chromatography (RP-HPLC) using both UV and electrospray mass spectrometric detection. The tryptic digest from either Process 1 or Process 2 was loaded onto an ODS-1 nonporous silica column (Micra, 1.5 μm; 53×4.6 mm) using a standard HPLC system in a mobile phase containing water, acetonitrile and trifluoroacetic acid. The eluting peptides were detected by UV at 214 nm (FIG. 2) and by positive-ion electrospray mass spectrometry. The major difference between the two chromatograms for Process 1 and Process 2 is the reduction in peak area of a peak obvious in the Process I trace (peak at 8.2 min; FIG. 2). This peak corresponds to the T1 peptide, residues 140. This observation is expected since the source of this peptide if from the intact CG53135-05, which is in greater abundance in the Process I material (peak 3, FIG. 1).

Bioassay

The biological activity of CG53135-05 related species collected from the 4 peaks identified by LC and MS was measured by treatment of serum-starved cultured NIH 3T3 murine embryonic fibroblast cells with various doses of the isolated CG53135-05 related species and measurement of incorporation of bromodeoxyuridine (BrdU) during DNA synthesis. For this assay, cells were cultured in Dulbecco's modified Eagle's medium supplemented with 10% fetal bovine serum. Cells were grown in 96-well plates to confluence at 37° C. in 10% CO₂/air and then starved in Dulbecco's modified Eagle's medium for 24-72 hours. CG53135-05-related species were added and incubated for 18 hours at 37° C. in 10% CO₂/air. BrdU (10 mM final concentration) was added and incubated with the cells for 2 hours at 37° C. in 10% CO₂/air. Incorporation of BrdU was measured by enzyme-linked immunosorbent assay according to the manufacturer's specifications (Roche Molecular Biochemicals, Indianapolis, Ind.).

Peak 4 was not included in this assay since insufficient material was collected (Peak 4 is less than 3% of the total peak area for CG53135-05). CG53135-05 and material collected from all 3 remaining fractions (i.e. Peak 1, 2, and 3) induced DNA synthesis in NIH 3T3 mouse fibroblasts in a dose-dependent manner (Table 7). The PI₂₀₀ was defined as the concentration of protein that resulted in incorporation of BrdU at 2 times the background. CG53135-05 and CG53135-05 related species recovered from all 3 measurable peaks demonstrated similar biological activity with a PI₂₀₀ of 0.7-11 ng/mL (Table 7).

TABLE 7
Biological Activity of CG53135-05 E. coli purified
product (DEV10): Induction of DNA Synthesis

| PI₂₀₀(ng/mL) | CG53135-05 (DEV 10) | Peak 1 | Peak 2 | Peak 3 |
|---|---|---|---|---|
| | 1.0 | 0.7 | 11 | 8.6 |

### 6.4 Example 4

### Stimulation of Bromodeoxyuridine Incorporation by CG53135

A dose response experiment for incorporation of BrdU was carried out using human renal carcinoma cells (786-0; American Type Culture Collection, Manassas, Va.). 293-EBNA cells (Invitrogen) were transfected using Lipofectamine 2000 according to the manufacturer's protocol (Life Technologies, Gaithersburg, Md.). Cells were supplemented with 10% fetal bovine serum (FBS; Life Technologies) 5 hours post-transfection. To generate protein for the assays, cells were washed and fed with Dulbecco's modified Eagle medium (DMEM; Life Technologies) 18 hours post-transfection. After 48 hours, the media was discarded and the cell monolayer was incubated with 100 μM suramin (Sigma, St. Louis, Mo.) in 0.5 ml DMEM for 30 min at 4° C. The suramin-extracted conditioned media was then removed, clarified by centrifugation (5 min; 2000×g), and subjected to TALON metal affinity chromatography according to the manufacturer's instructions (Clontech, Palo Alto, Calif.) taking advantage of the carboxy-terminal polyhistidine tag. Retained fusion protein was released by washing the column with imidazole.

To generate control protein, 293-EBNA cells were transfected with pCEP4 plasmid (Invitrogen) and subjected to the purification procedure outlined above.

Isolated CG53135 protein was tested for its ability to induce DNA synthesis in a bromodeoxyuridine (BrdU) incorporation assay. 786-0 cells were cultured in 96-well plates to approximately 100% confluence, washed with DMEM, and serum-starved in DMEM for 24 hours. Isolated CG53135 protein or control protein was then added to the cells for 18 hours. The BrdU assay was performed according to the manufacturer's specifications (Roche Molecular Biochemicals, Indianapolis, Ind.) using a 5 hour BrdU incorporation time.

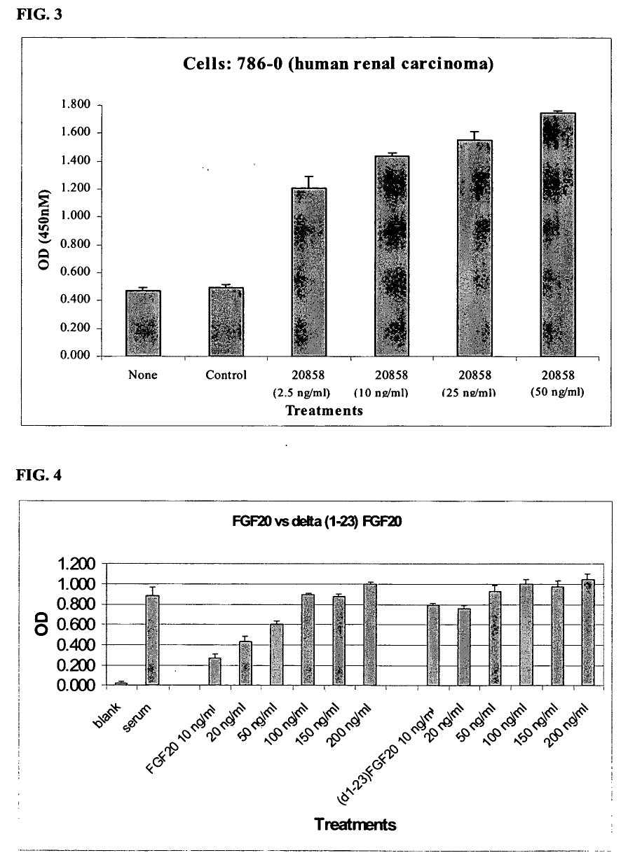

The results are shown in FIG. 3, in which CG53135 is designated “20858”. It is seen that CG53135 stimulates proliferation of renal carcinoma cells by more than 4-fold over controls, with a half-effective dose being about 2.5 ng/mL.

A vector expressing amino acids 24-211 of FGF-20, referred to as CG53135-17 (SEQ ID NOs:31 and 32), was prepared. The incorporation of BrdU by NIH 3T3 cells treated with conditioned medium obtained using the vector incorporating this truncated form was compared to the incorporation in response to treatment with conditioned medium using a vector encoding full length FGF-20. This experiment was carried out as described above.

The results are shown in FIG. 4. It is seen that CG53135-17 retains high activity at the lowest concentration tested, 10 ng/mL.

### 6.5 Example 5

### Receptor Binding Specificity of FGF-20.

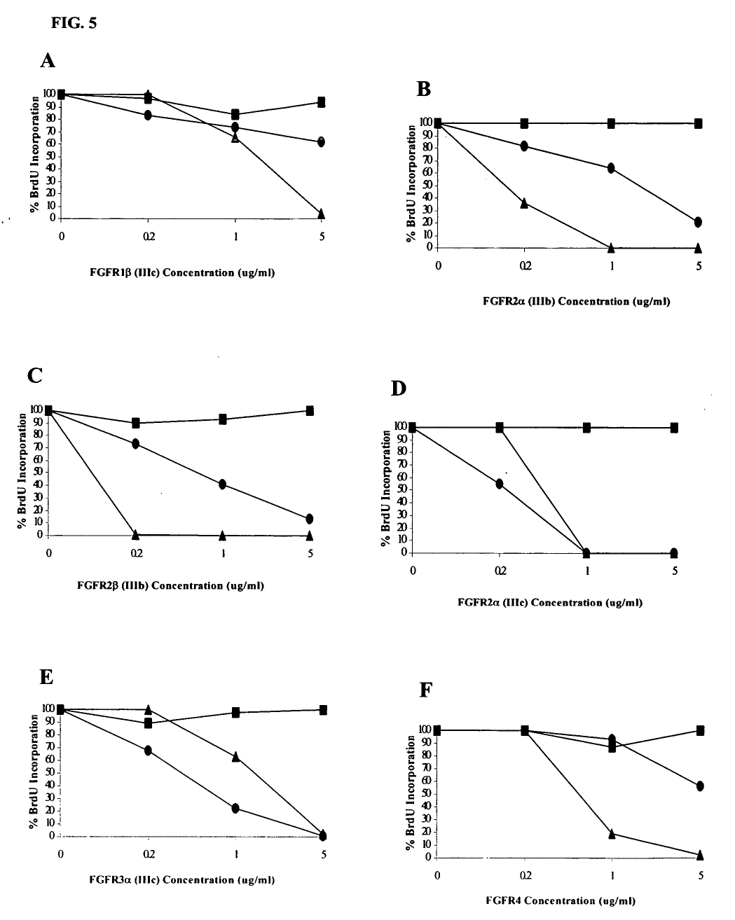

To determine the receptor binding specificity of FGF-20, we examined the effect of soluble FGF receptors (FGFRs) on the induction of DNA synthesis in NIH 3T3 cells by recombinant FGF-20. Four receptors have been identified to date (Klint P and Claesson-Welsh L. Front. Biosci., 4: 165-177, 1999; Xu X, et al. Cell Tissue Res., 296: 33-43, 1999). Soluble receptors for FGFR1β(IIIc), FGFR2a(IIIb), FGFR2β(IIIb), FGFR2a(IIIc), FGFR3a(IIIc) and FGFR4 were utilized. It was found that soluble forms of each of these FGFRs were able to specifically inhibit the biological activity of FGF-20 (see FIG. 5). Complete or nearly complete inhibition was obtained with soluble FGFR2a(IIIb), FGFR2β(IIIb), FGFR2a(IIIc), and FGFR3a(IIIc), whereas partial inhibition was achieved with soluble FGFR1β(IIIc) and FGFR4. None of the soluble receptor reagents interfered with the induction of DNA synthesis by PDGF-BB (control), thereby demonstrating their specificity. The integrity of each soluble receptor reagent was demonstrated by showing its ability to inhibit the induction of DNA synthesis by aFGF (acidic FGF), a factor known to interact with all of the FGFRs under analysis.

### 6.6 Example 6

### Wound Repair Test

In vitro cell culture (apply to Sections 6.6-6.9): The human colon cancer cell line Caco2, HT29 and THP-1 cells were obtained from the American Type Culture Collection (Rockville, Md.), HT-29 MTX were provided by Dr. Lesuffler, INSERM, Dillejuis, France. These cell lines (Caco2, HT-29 and HT-29MTX) were grown as described previously. THP-1 cell lines were grown in RPMI-1640 medium (Life Technologies, Gaithersburg, Md.) with 10% fetal bovine serum, 100 units/ml of antibiotics/antimycotics (Life Technologies, Gaithersburg, Md.).

An in vitro healing assay was performed using a modified method. Briefly, reference lines were drawn horizontally across the outer bottom of 24-well plates. HT-29 and Caco-2 cells were seeded and grown to confluence, then incubated with media containing 0.1% FBS for 24 hours.

Linear wounds were made with a sterile plastic pipette tip perpendicular to the lines on the bottom of the well. Isolated CG53135-05 E. coli purified product (100 ng/ml) was then added. The size of the wound was measured at three predetermined locations at various times after wounding (0, 6, 20 and 24 hours). The closure of the wounds was measured microscopically at 20× magnification over time, and the mean percentage of wound closure was calculated relative to baseline values (time 0). To investigate whether the effect of FGF-20 on cell restitution is involved with TGF-β and ITF pathway, anti-TGFβ antibody (R&D system, Minneapolis, Minn.) and polyclonal anti-ITF antibody (a gift from D K Podolsky, Harvard Medical School, Boston, Mass.) were used.

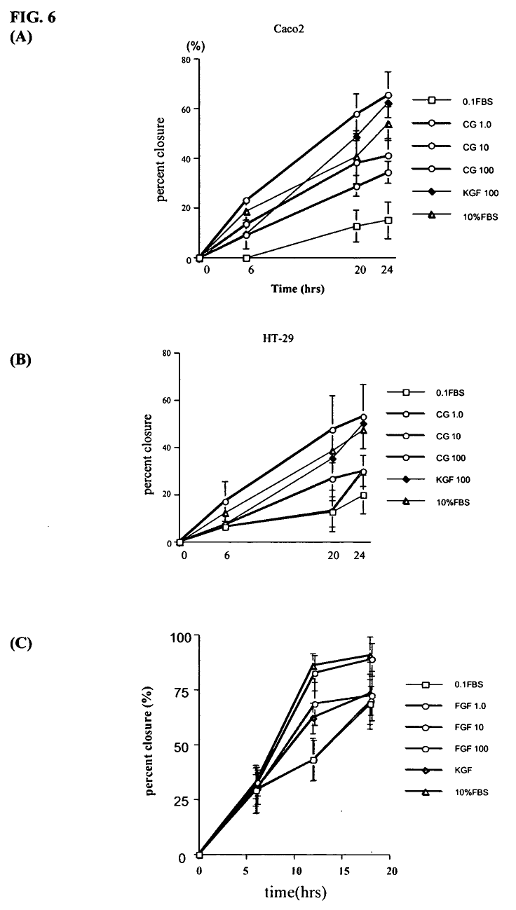

FIG. 6 show the effect of FGF-20 in the closure of wounds in various human cell lines. There is a dose dependent increase in the effectiveness of FGF-20 in the closure of wounds in all the cell lines tested, demonstrating the role of FGF-20 in wound repair.

### 6.7 Example 7

### Reverse Transcriptase-Polymerase Chain Reaction (RT-PCR)

Total RNA from cell lines and the colonic tissue was prepared using TRIzol reagent (Invitrogen) according to the manufacture's instructions. RNA was reverse transcribed using 2 μg of total RNA, 15U of RNA inhibitors, 1× first strand buffer (Life technologies, Long Island, N.Y.), 5 mM dNTP (Pharmacia, Uppsal, Sweden), 125 pmol random hexamer primers (Pharmacia), and 125 U of Moloney murine leukemia virus RT (Life Technologies) in a final volume of 25 ul. The reaction was carried out for 1 hour at 39° C. followed by 7 minutes at 93° C. and 1 minutes at 24° C. and then slowly cooled to 4° C. for 20 minutes. PCR was carried out in a volume of 50 μl containing 5 μl of RT mixture, 1× Thermos aquaticus (Taq) buffer, 5 pmol of each primer, 2.5 mM dNTP, and 1 U of Taq polymerase.

The sequence of primers used were as follows:
human COX-2 sense; 5′-AGATCATCTCTGCCTGAGTATCTT-3′ (SEQ ID NO: 42),
human COX-2 antisense: 5′-TTCAAATGAGATTGTGGGAAAATTGCT-3′ (SEQ ID NO: 43),
human Intestinal trefoil factor (ITF) sense: 5′-GTGCCAGCCMGGACAG-3′, (SEQ ID NO: 44),
human ITF antisense: 5′-CGTTAAGACATCAGCCTCCAG-3′, (SEQ ID NO: 45),
human PPAR-? sense: 5′-TCTCTCCGTMTGGMGACC-3′ (SEQ ID NO: 46),
human PPAR-? antisense: 5′-GCATTATGAGACATCCCCAC-3′ (SEQ ID NO: 47),
human β-actin sense: 5′-CCMCCGCMGMGATGA-3′ (SEQ ID NO: 48),
human β-actin antisense: 5′-GATCTTCATGAGGTAGTCAGT-3′ (SEQ ID NO: 49),
mouse COX-2 sense: 5′-GCAAATCCTTGCTGTTCCMTC-3′ (SEQ ID NO: 50),
mouse COX-2 antisense: 5′-GGAGAAGGCTTCCCAGCTTTTG-3′ (SEQ ID NO: 51),
mouse ITF sense: 5′-GMGTTTGCGTGCTGCCATGGAG-3′ (SEQ ID NO: 52),
mouse ITF antisense: 5′-CCGCAATTAGMCAGCCTTGTG-3′ (SEQ ID NO: 53),
mouse IL-10 sense: 5′-CTCTTACTGACTGGCATGAGGATC-3′ (SEQ ID NO: 54),
mouse IL-10 antisense: 5′-CTATGCAGTTGATGMGATGTCAMTT-3′ (SEQ ID NO: 55),
mouse G3PDH sense: 5′-CGGTGCTGAGTATGTCGTGGAGTCT-3′ (SEQ ID NO: 56),
mouse G3PDH antisense: 5′-GTTATTATGGGGGTCTGGGATGGM-3′ (SEQ ID NO: 57).

PCR was carried out in a Perkin-Elmer 9600 cycler set for 20-40 cycles to assess linearity of the amplification. The PCR products were electrophoresed on 2% tris-acetate and EDTA agarose gels containing gel star fluorescent dye (FMC Corporation, Philadelphia, Pa.). A negative from the gels was taken with Alphalmager 2000 (Alpha Innotech Corporation, Calif.) and relative abundance of RT-PCR transcript was assessed by Adobe photoshop 3.0.4 soft ware, normalized to the density of β-actin and G3PDH transcript.

Expression of some protective genes was also detected by mRNA expression in cell lines or cells isolated from mice (C57BL6) using standard procedures.

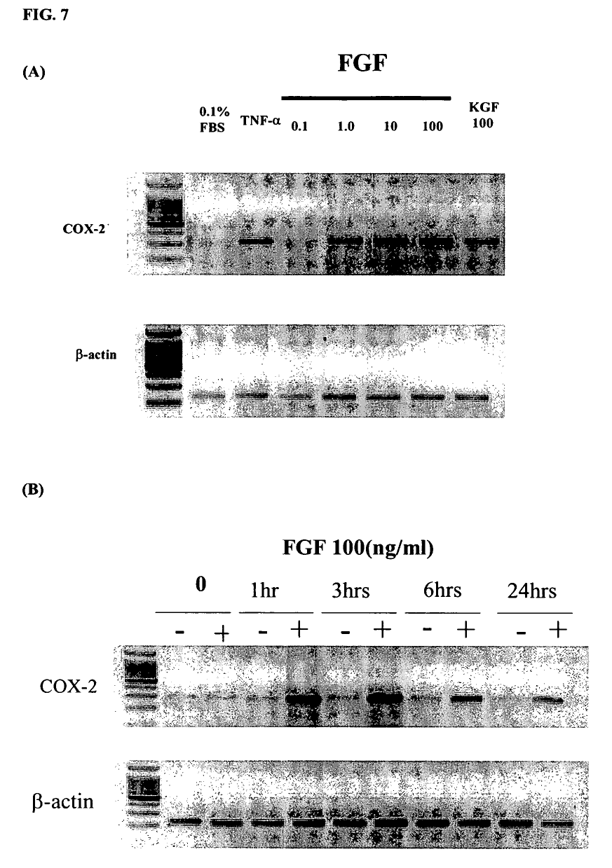

COX-2 gene expression in HT29 cell line in the presence of CG53135 was dose dependent, showing highest expression when induced by 100 ng/ml of CG53135-05 E. coli purified product (FIG. 7). At this concentration, the gene expression was higher at 1 hour and 3 hour time periods of incubation and decreased thereafter at 6 hour and 24 hour.

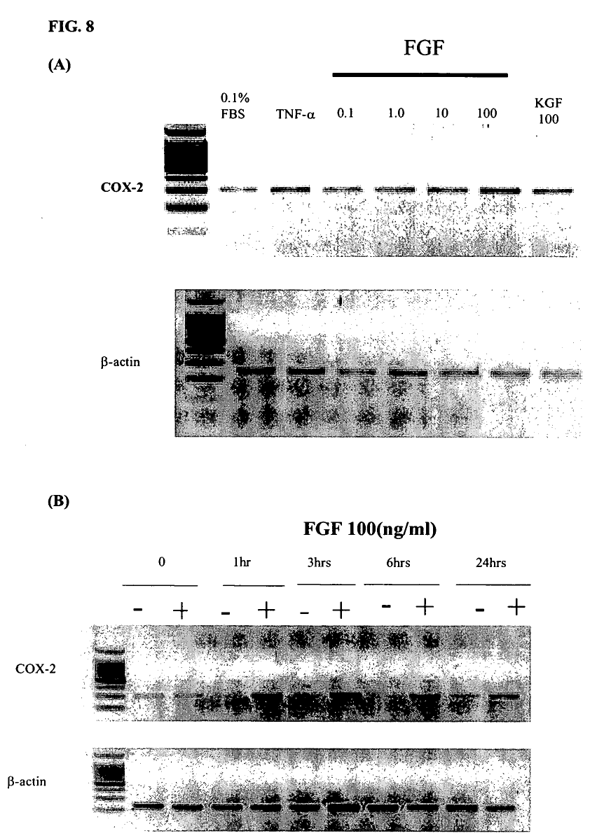

COX-2 gene expression in Caco2 cell line was high when stimulated with 10 ng/ml of CG53135 as seen in FIG. 8. Increased expression of COX2 was detected at 1, 3 and 6 hours after incubation with 100 ng/ml of CG53135 E. coli purified product.

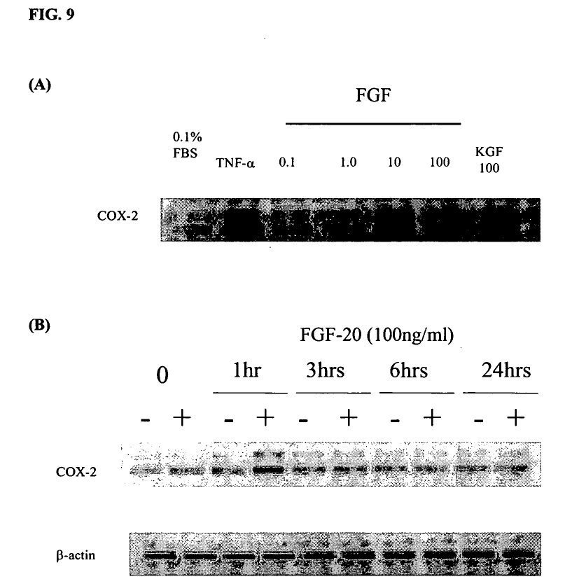

Expression of COX-2 in IEC-6 cell line showed a dose dependent increase in the presence of CG53135 (FIG. 9). Increased expression of COX-2 was detected at 1 hour after incubation with 100 ng/ml of CG53135-05 E. coli purified product.

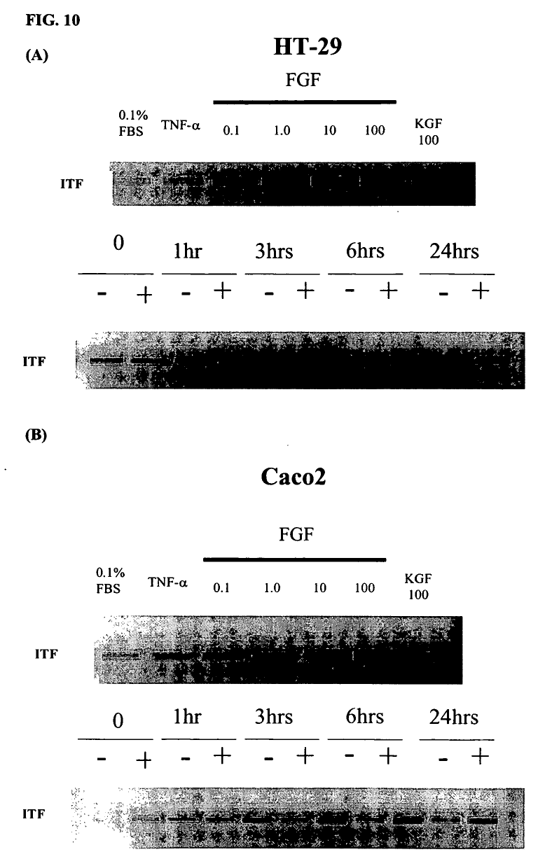

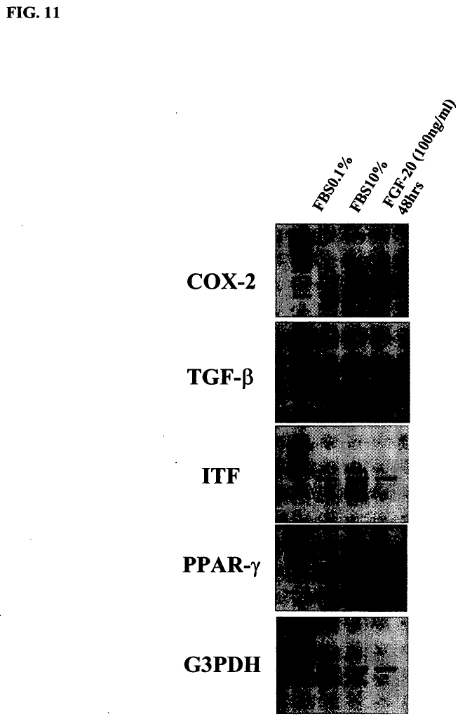

Expression of Intestinal Trefoil factor (ITF) in HT-29 and Caco2 cell lines, in the presence of CG53135, is shown in FIG. 10. Results show dose and time dependent increase in expression of ITF in both HT-29 and Caco2 cells when stimulated by FGF-20. FIG. 11 reiterates that COX-2 is expressed in HT-29 cells. In addition, TGF-β, ITF, PPAR-? expression is also shown in FIG. 11.

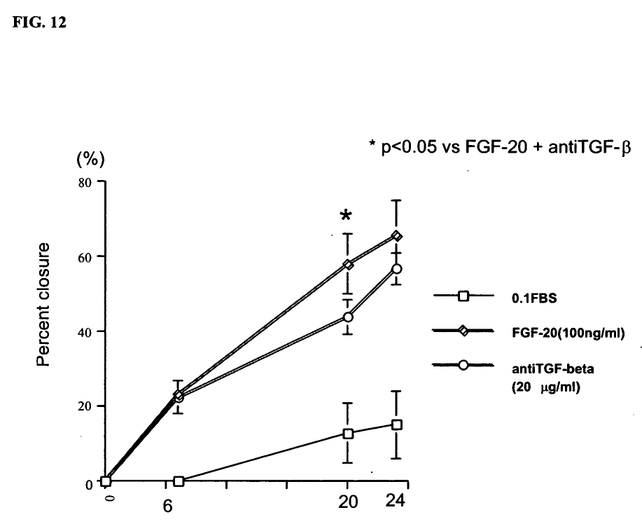

The results presented suggest that CG53135 plays a key role in mucosal repair possibly by inducing COX-2 and ITF genes. Based on the data, that FGF-20 induces TGF-β expression, wound repair in Caco-2 cells was tested as described in Example 6, in the presence of antiTGF-β antibody (20 μg/ml). FIG. 12 shows that Epithelial Restitution by XG53135 is mediated in part by TGF-β pathway (p<0.05 vs CG53135 E. coli purified product+antiTGF-β).

### 6.8 Example 8

### Transcription Pathway Assays

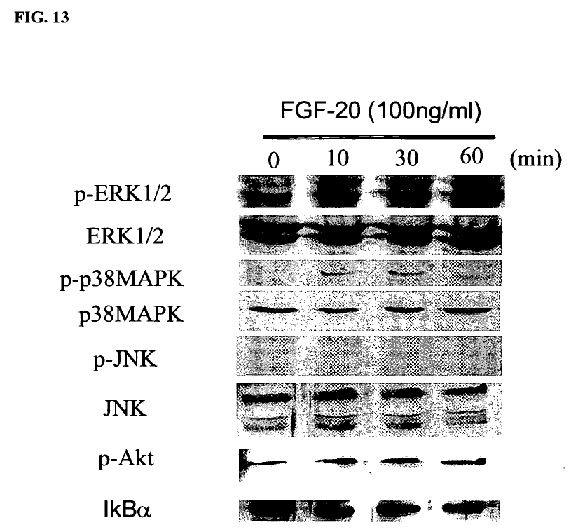

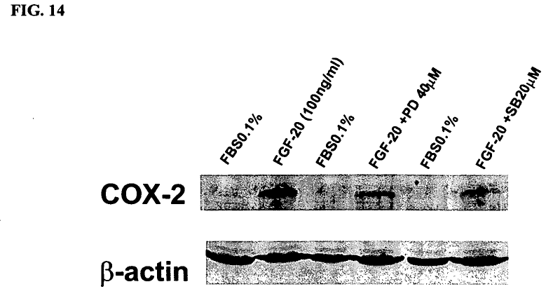

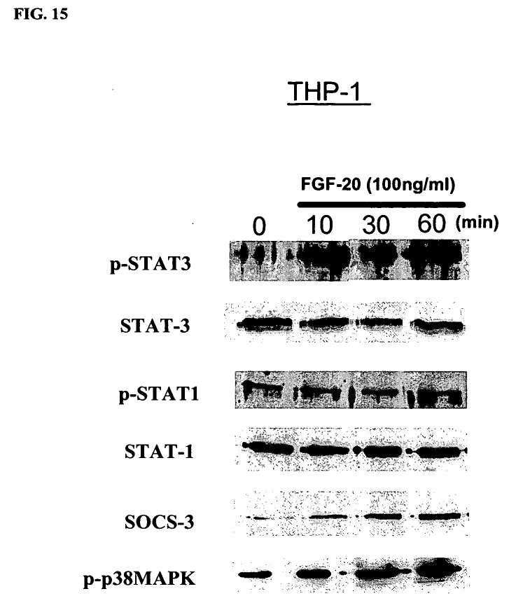

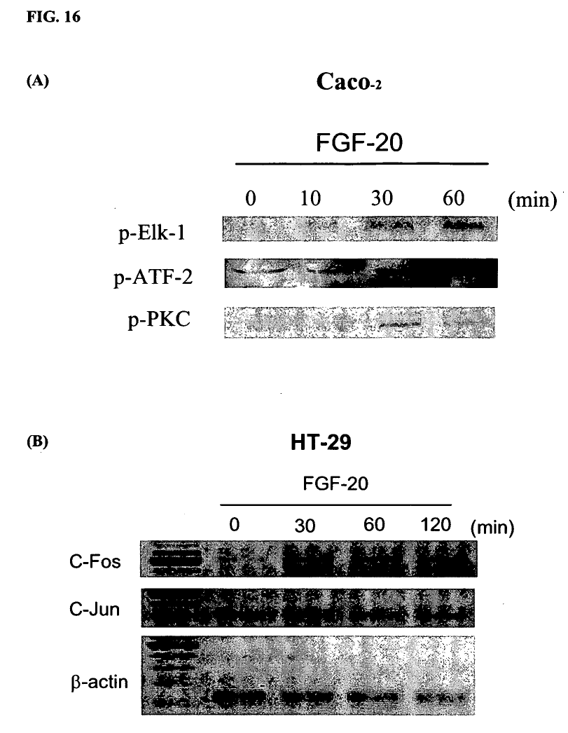

Signal transduction was considered a possible mechanism for inducing COX-2 expression in epithelial cells, upon stimulation with CG53135. Various kinases were tested for their expression in the presence of 10 ng/ml of CG53135 E. coli purified product, in Caco2 cells. The results indicated that phosphorylated MAPK (p-p38MAPK) was induced in the presence of CG53135, while no other kinase tested, showed any significant induction (FIG. 13). Also IkBa expression demonstrated moderate degradation in the presence of CG53135. In addition, FIG. 14 demonstrates that inhibitors of Erk and MAPK decreased COX-2 expression in the presence of FGF-20, in Caco2 cells. Furthermore, expression of kinases was analyzed in THP-1 macrophage cell line, in the presence of 10 ng/ml of CG53135-05 E. coli purified product (FIG. 15). The results demonstrated increased expression of phosphorylated STAT3, p-p38MAPK and SOCS-3 genes. Also FIG. 16 shows increased expression of phosphorylated Elk-1, ATF-2 and minimal induction of phosphorylated Protein Kinase C in Caco-2 cells in the presence of CG53135. In HT-29 cells, C-Fos and C-Jun were induced, when cultured with CG53135 (FIG. 16).

### 6.9 Example 9

### Adenoviral Infection and Plasmid Transfection and Reporter Gene Assay

Caco-2 and HT-29 were infected overnight with Ad5 kB-LUC, which consists of three consensus NF-kB binding sites that was linked to luciferase. Ad5LacZ containing the E. coli beta-galactosidase were used as viral negative control. The adenovirus were washed off and fresh medium containing serum (without antibiotics was added. Cells were stimulated with CG53135 E. coli purified product (100 ng/ml), IL-β (5 ng/ml) and TNF-a (10 ng/ml). HT-29 were transfected using LipofectAMINE reagent (Invitrogen) as described previously. Mouse ITF promoter plasmid (1 μg, generous gift of D. K Podolsky, Harvard, Boston, Mass.) was transfected. Transfected cells were incubated overnight after the DNA/LipofectAMINE was replaced with serum containing media. Cells were then stimulated with CG53135 E. coli purified product (100 ng/ml) for 24 hours. Cell extracts were prepared using enhanced luciferase assay reagents (Analytical Luminescence, San Diego, Calif.), and Luciferase assay was performed on a Monolight 2010 luminometer for 20 s (Analytical Luminescence, San Diego, Calif.), and results were normalized for extract protein concentrations measured with the Bio-Rad protein assay kit.

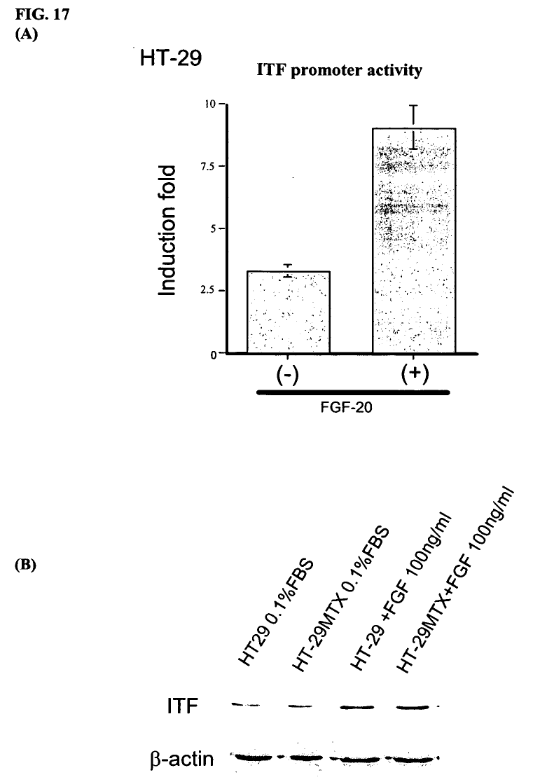

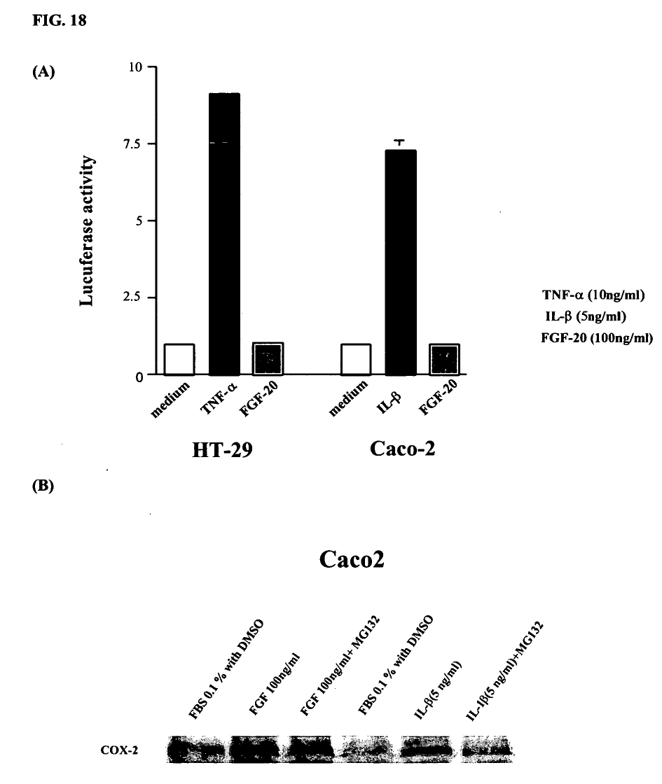

ITF promoter activity in HT-29 cells was assayed. The results show that there is more than 2-fold induction of ITF in the presence of CG53135 at a concentration of 100 ng/ml (FIG. 17), suggesting that CG53135 activates ITF transcription in HT-29 cells. Furthermore, CG53135 does not activate NF-kB expression in either HT-29 or Caco-2 cell lines as seen in FIG. 18. Thus NF-kB may not have a role inducing COX-2 expression in the presence of CG53135. This has been further conclusively shown in FIG. 18, where MG132, a proteosome inhibitor does not block COX-2 induction in Caco-2 cells.

### 6.10 Example 10

### Cellular Proliferation Responses with CG53135 (Studies L-117.01 and L-117.02)

Experiments were performed to evaluate the proliferative response of representative cell types to CG53135, e.g., a full-length tagged variant (CG53135-01), a deletion variant (CG53135-02), and a full-length codon-optimized untagged variant (CG53135-05).

Materials and Methods:

Heterologous Protein Expression: CG53135-01 (batch 4A and 6) was used in these experiments. Protein was expressed using Escherichia coli (E. coli), BL21 (Novagen, Madison, Wis.), transformed with full-length CG53135-01 in a pETMY-hFGF20X/BL21 expression vector. Cells were harvested and disrupted, and then the soluble protein fraction was clarified by filtration and passed through a metal chelation column. The final protein fraction was dialyzed against phosphate buffered saline (PBS) plus 1 M L-arginine. Protein samples were stored at −70° C.

CG53135-02 (batch 1 and 13) was also used in these experiments. Protein was expressed in E. coli, BLR (DE3) (Novagen), transformed with the deletion variant CG53135-02 inserted into a pET24a vector (Novagen). A research cell bank (RCB) was produced and cell paste containing CG53135-02 was produced by fermentation of cells originating from the RCB. Cell membranes were disrupted by high-pressure homogenization, and lysate was clarified by centrifugation. CG53135-02 was purified by ion exchange chromatography. The final protein fraction was dialyzed against the formulation buffer (100 mM citrate, 1 mM ethylenediaminetetraacetic acid (EDTA), and 1 M L-arginine).

CG53135-05, DEV10, which were also used in these experiments, was prepared by Cambrex Biosciences (Hopkinton, Mass.) according to Process 1 as described in Section 6.18.1, infra.

BrdU Incorporation: proliferative activity was measured by treatment of serum-starved cultured cells with a given agent and measurement of BrdU incorporation during DNA synthesis. Cells were cultured in respective manufacturer recommended basal growth medium supplemented with 10% fetal bovine serum or 10% calf serum as per manufacturer recommendations. Cells were grown in 96-well plates to confluence at 37° C. in 10% CO₂/air (to subconfluence at 5% CO₂ for dedifferentiated chondrocytes and NHOst). Cells were then starved in respective basal growth medium for 24-72 hours. CG53135 protein purified from E. coli or pCEP4/Sec or pCEP4/Sec-FGF 20× enriched conditioned medium was added (10 μL/100 μL of culture) for 18 hours. BrdU (10 μM final concentration) was then added and incubated with the cells for 5 hours. BrdU incorporation was assayed according to the manufacturer's specifications (Roche Molecular Biochemicals, Indianapolis, Ind.).

Growth Assay: growth activity was obtained by measuring cell number following treatment of cultured cells with a given agent for a specified period of time. In general, cells grown to ˜20% confluency in 6-well dishes were treated with basal medium supplemented with CG53135 or control, incubated for several days, trypsinized and counted using a Coulter Z1 Particle Counter.

Results:

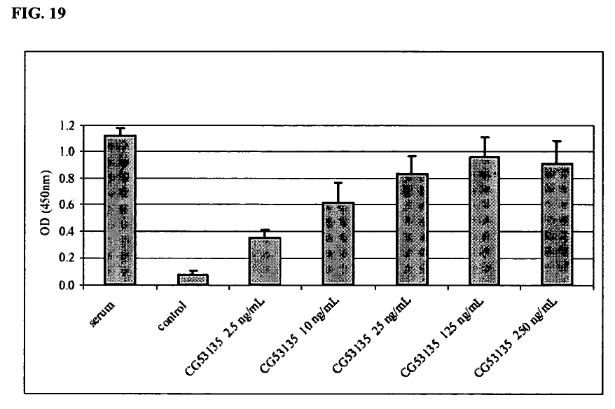

Proliferation in Mesenchymal Cells: to determine if recombinant CG53135 could stimulate DNA synthesis in fibroblasts, a BrdU incorporation assay was performed using CG53135-01 treated NIH 3T3 murine embryonic lung fibroblasts. Recombinant CG53135-01 induced DNA synthesis in NIH 3T3 mouse fibroblasts in a dose-dependent manner (FIG. 19). DNA synthesis was generally induced at a half maximal concentration of ˜10 ng/mL. In contrast, treatment with vehicle control purified from cells did not induce any DNA synthesis.

CG53135-01 also induced DNA synthesis in other cells of mesenchymal origin, including CCD-1070Sk normal human foreskin fibroblasts, MG-63 osteosarcoma cell line, and rabbit synoviocyte cell line, HIG-82. In contrast, CG53135-01 did not induce any significant increase in DNA synthesis in primary human osteoblasts (NHOst), human pulmonary artery smooth muscle cells, human coronary artery smooth muscle cells, human aorta smooth muscle cells (HSMC), or in mouse skeletal muscle cells.

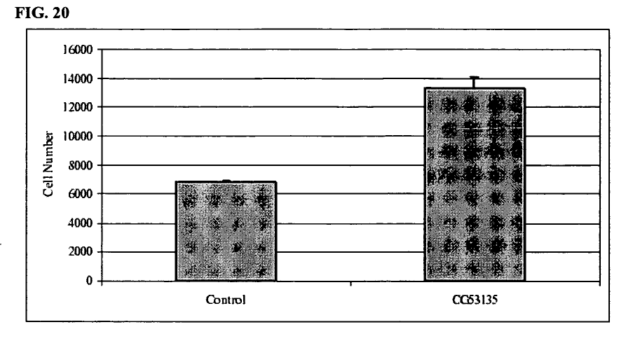

To determine if recombinant CG53135-01 sustained cell growth, NIH 3T3 cells were cultured with 1 μg CG53135-01 or control for 48 hours and then counted (FIG. 20). CG53135 induced an approximately 2-fold increase in cell number relative to control in this assay. These results show that CG53135 acts as a growth factor.

Proliferation of Epithelial Cells: to determine if recombinant CG53135 can stimulate DNA synthesis and sustain cell growth in epithelial cells, a BrdU incorporation assay was performed in representative epithelial cell lines treated with CG53135. Cell counts following protein treatment were also determined for some cell lines.

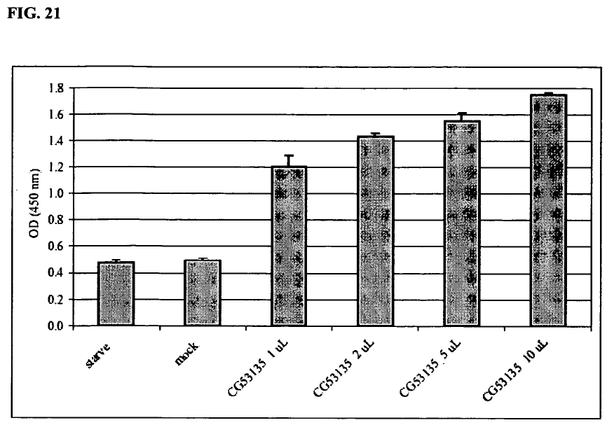

CG53135 was found to induce DNA synthesis in the 786-0 human renal carcinoma cell line in a dose-dependent manner (FIG. 21). In addition, CG53135-01 induced DNA synthesis in other cells of epithelial origin, including CCD 1106 KERTr human keratinocytes, Balb MK mouse keratinocytes, and breast epithelial cell line, B5589.

Proliferation of Hematopoietic Cells: no stimulatory effect on DNA synthesis was observed upon treatment of TF-1, an erythroblastic leukemia cell line with CG53135-01. These data suggest that CG53135-01 does not induce proliferation in cells of erythroid origin. In addition, Jurkat, an acute T-lymphoblastic leukemia cell line, did not show any response when treated with CG53135-01, whereas a robust stimulation of BrdU incorporation was observed with serum treatment.

Effects of CG53135 on Endothelial Cells: protein therapeutic agents may inhibit or promote angiogenesis, the process through which endothelial cells differentiate into capillaries. Because CG53135 belongs to the fibroblast growth factor family, some members of which have angiogenic properties, the antiangiogenic or pro-angiogenic effects of CG53135 on endothelial cell lines were evaluated. The following cell lines were chosen because they are cell types used in understanding angiogenesis in cancer: HUVEC (human umbilical vein endothelial cells), BAEC (bovine aortic endothelial cells), HMVEC-d (human endothelial, dermal capillary). These endothelial cell types undergo morphogenic differentiation and are representative of large vessel (HUVEC, BAEC) as well as capillary endothelial cells (HMVEC-d).

CG53135-01 treatment did not alter cell survival or have stimulatory effects on BrdU incorporation in human umbilical vein endothelial cells, human dermal microvascular endothelial cells or bovine aortic endothelial cells. Furthermore, CG53135-01 treatment did not inhibit tube formation, an important event in formation of new blood vessels, in HUVECS. This result suggests that CG53135 does not have anti-angiogenic properties. Finally, CG53135-01 had no effect on VEGF induced cell migration in HUVECs, suggesting that it does no play a role in metastasis.

The above described experiments were also performed using CG53135-02 and CG53135-05 protein products, and the results are summarized in the Conclusion section below.

Conclusions

Recombinant CG53135-01 induces a proliferative response in mesenchymal and epithelial cells in vitro (i.e., NIH 3T3 mouse fibroblasts, CCD-1070 normal human skin fibroblasts, CCD-1106 human keratinocytes, 786-0 human renal carcinoma cells, MG-63 human osteosarcoma cells and human breast epithelial cells), but not in human smooth muscle, erythroid, or endothelial cells. Like CG53135-01, CG53135-02 and CG53135-05 also induce proliferation of mesenchymal and epithelial cells. In addition, CG53135-02 (but not CG53135-01 nor CG53135-05) induces proliferation of endothelial cells.

### 6.11 Example 11

### Activity of CG53135 in Mouse Model for Inflammatory Bowel Disease

This experiment examines activities of CG53135 in a widely recognized animal model for inflammatory bowel disease, the mouse dosed with the sodium form of dextran sulfate.

Materials and Methods

Colitis Study Design: Normal female Balb/c mice (Harlan Labs), 6-8 weeks old weighing approximately 20 g, were housed 3-5 animals per cage in polycarbonate cages with filter tops and given food (Harlan Teklad mouse chow) and tap water ad libitum. Mice were acclimated for 6 days (Day-7 through Day-1) and then given water orally (po) ad libitum containing 5% dextran sulfate sodium (DSS) or control water ad libitum for 7 days (Day 0 through Day 6). DSS (Spectrum Chemicals, Gardena Calif.) was made as a 5% solution in tap water; DSS was made every other day and stored at 4° C. Mice were divided into 4 treatment groups (Table 8). On Day 0, daily intraperitoneal (ip) treatments with vehicle (1 M L-arginine in phosphate buffered saline) or protein (CG53135 or CG52053 (another protein interested), 5 mg/kg) were initiated and continued each morning through Day 6. On Day 7, mice were sacrificed by exposure to carbon dioxide (CO₂).

TABLE 8
Treatment Groups

| Group | N^{a} | Treatment |
|---|---|---|
| 1 | 5 | Normal control: no DSS water + vehicle ip |
| 2 | 10 | Disease control: DSS water po + vehicle ip |
| 3 | 10 | CG53135: DSS water po + 5 mg/kg CG 53135 ip |
| 4 | 5 | CG52053: DSS water po + 5 mg/kg CG52053 ip |

^{a}N = number of animals per group

Protein production: The cDNA for CG53135-01 was identified and cloned into the pRSET vector (Invitrogen) to provide the vector pETMY-CG53135-01. The gene product of this construct provides a polypeptide incorporating (His)₆-(enterokinase cleavage site)-(multicloning site) at the N-terminal end of the polypeptide; in addition, in this construct, the CG53135 sequence begins with the Ala at position 2 FGF-20 (SEQ ID NO:2). This vector was transformed into Escherichia coli. The E. coli cells were grown up to 10 L scale and infected with CE6 phage to produce the recombinant CG53135. The recombinant protein was purified by disrupting the E. coli cells in a microfluidizer and extraction with 1M L-arginine solution, followed by multiple metal affinity chromatography steps. The final purified protein was dialyzed into phosphate buffered saline containing 1M L-arginine. Protein purity was determined by SDS-PAGE analysis and identities were confirmed by Western blot analysis. Activity of proteins was determined by BrdU incorporation assay (Roche Molecular Biochemicals) using a 5 hour incorporation time and NIH 3T3 cells.

Body weights were measured daily and at termination on day 7. Additional parameters measured at necropsy included colon length, colon weight and spleen weights. Colon and spleen were collected into formalin for histopathologic evaluation.

Colon content was scored at necropsy according to the following criteria:

0=normal to semi-solid stool, no blood observed
1=normal to semi-solid stool, blood tinged
2=semi-solid to fluid stool with definite evidence of blood
3=bloody fluid
   Pathology Methods

Three sections approximately 1 cm apart from the distal end (area that is most severely affected in this model) and 3 sections approximately 1 cm apart from the proximal end (less severely affected area) were processed for paraffin embedding, sectioned and stained with hematoxylin and eosin for pathologic evaluation.

For each section, submucosal edema was quantitated by measuring the distance from the muscularis mucosa to the internal border of the outer muscle layer. Inflammation (foamy macrophage, lymphocyte and PMN infiltrate) was assigned severity scores according to the following: Normal=0; Minimal=1; Mild=2; Moderate=3; Marked=4; and Severe=5. Splenic lymphoid atrophy was also scored by the above criteria.

The parameters reflecting epithelial cell loss/damage were scored individually using a % area involved scoring method: None=0; 1-10% of the mucosa affected=1; 11-25% of the mucosa affected=2; 26-50% of the mucosa affected=3; 51-75% of the mucosa affected=4; and 76-100% of the mucosa affected=5.

Parameters that were scored using % involvement included: Colon glandular epithelial loss—this includes crypt epithelial as well as remaining gland epithelial loss and would equate to crypt damage score; and Colon Erosion—this reflects loss of surface epithelium and generally was associated with mucosal hemorrhage (reflective of the bleeding seen clinically and at necropsy).

For each animal, 3 proximal (less severe lesions) and 3 distal (most severe lesion) areas were scored and the mean of the scores for each of the regions was determined. Group means and % inhibition from disease control were determined. By doing it this way (rather than summing the scores from various sections) one can look at the mean±SE for in individual parameter (represented by 3 sections) and equate it to a delineated severity. As an example, if the mean is 4 for gland epithelial loss one knows that 51-75% of the mucosa was devoid of epithelium.

The three important scored parameters (inflammation, glandular epithelial loss, erosion) were ultimately summed to arrive at a sum of histopathology score which indicates the overall damage and would have a maximum score of 15.

One final summation of proximal+distal summed scores was done to reflect the overall total colonic severity score.

Statistics: The mean and standard error (SE) for each treatment group was determined for each parameter scored; the data were compared to the data for the disease controls (Group 2) using a 2-tailed Student's t test with significance at p=0.05.

Results

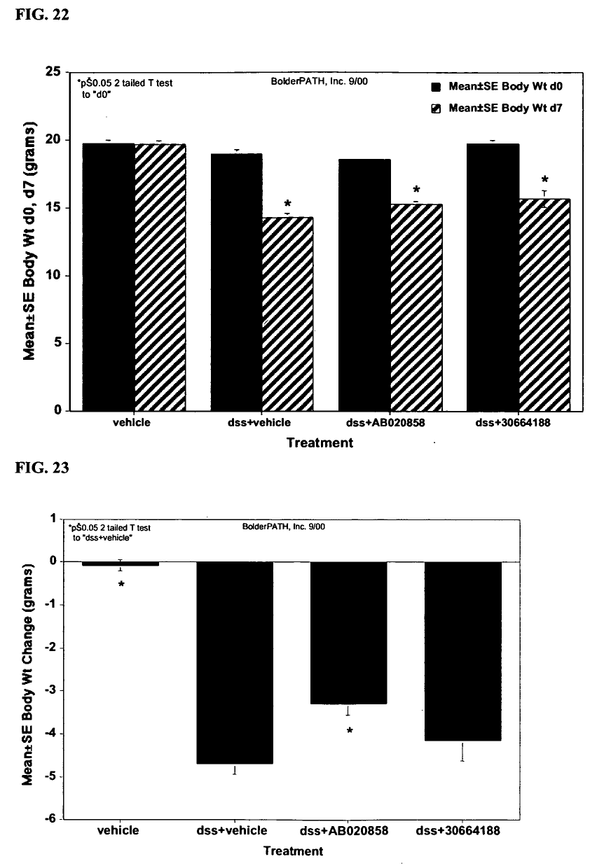

Live Phase, Necropsy and Organ Weight: All animals except DSS+vehicle control mouse 4 survived to study termination. Mouse 4 was found dead the morning of necropsy on day 7. DSS treatment-related changes in body weight were present by day 3 in all DSS treated mice. At study termination, DSS+vehicle controls had a 25% decrease in body weight (FIGS. 22, 23, and 24). A significant beneficial effect on DSS induced weight loss was seen in mice given CG53135, referred to as AB020858 (FIGS. 23 and 24).

Clinical evidence of bloody diarrhea was evident in all DSS+vehicle animals except animal 1. At necropsy all DSS controls had blood or blood tinged fluid in the colon. In contrast, mice treated with AB020858 (CG53135) generally had semi-solid stool and little evidence of blood. Similar findings occurred in mice treated with 30664188 (CG52053).

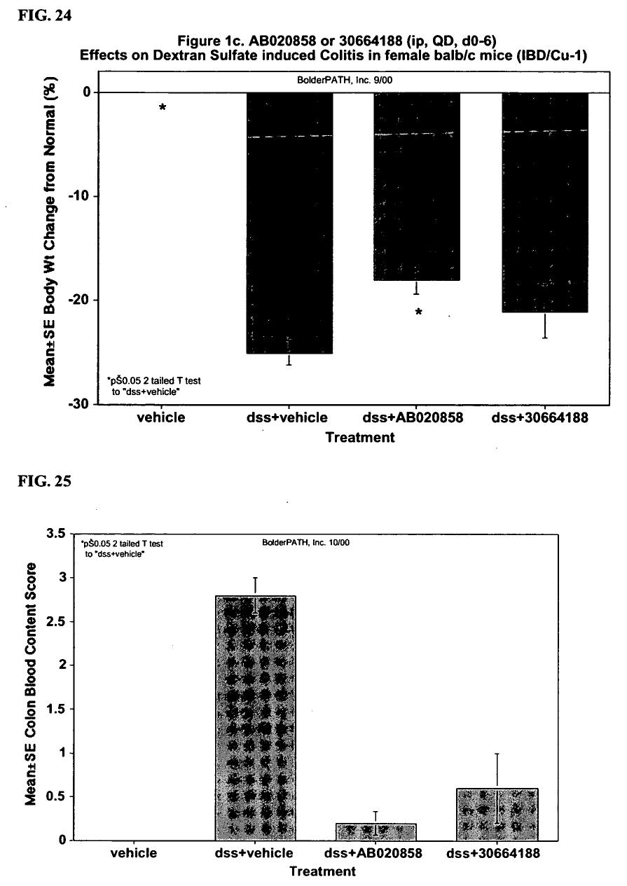

Colon content scores reflecting colonic hemorrhage were dramatically decreased (93%) in mice treated with AB020858 (CG53135) and (79%) in mice treated with 30664188 (CG52053) (FIG. 25).

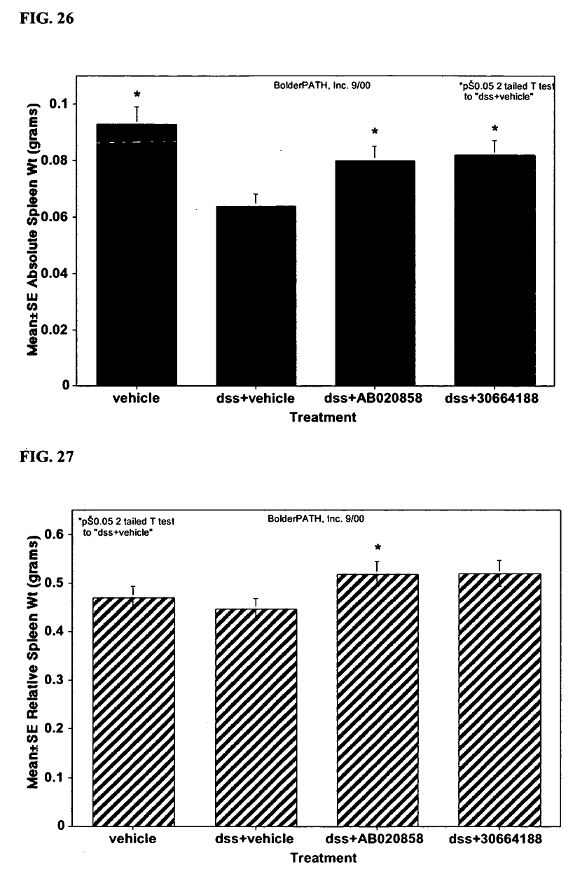

Absolute spleen weights (FIGS. 26 and 27) were decreased approximately 30% in mice treated with vehicle. Treatment with AB020858 (CG53135) resulted in 55% reduction of the DSS-induced losses in spleen weights. Treatment with 30664188 (CG52053) reduced the splenic weight losses by 62%.

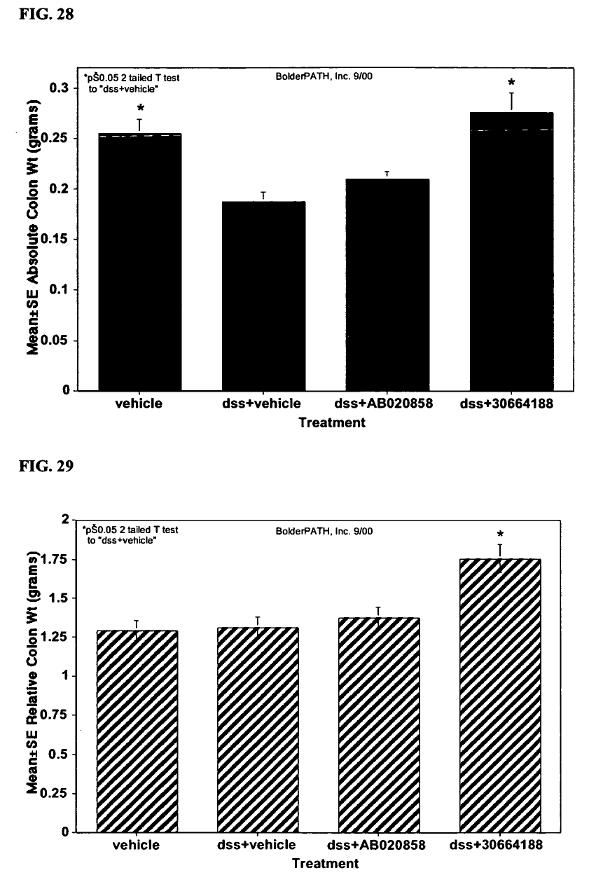

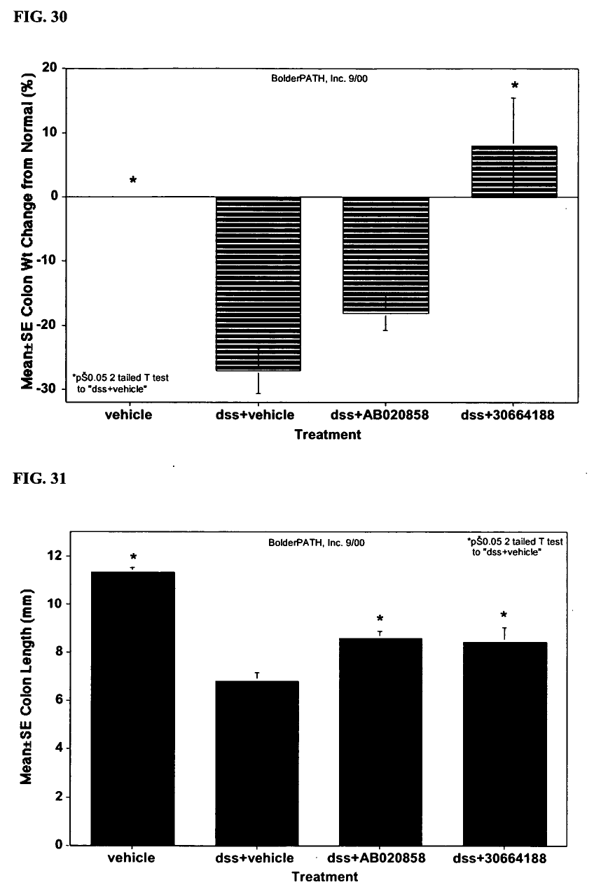

Absolute colon weights (FIGS. 28 and 29) were decreased approximately 26% in mice treated with vehicle. Treatment with AB020858 (CG53135) resulted in slight but not significant reduction of the DSS-induced changes in colon weights. Treatment with 30664188 (CG52053) reversed the colon weight decreases (FIGS. 29 and 30).

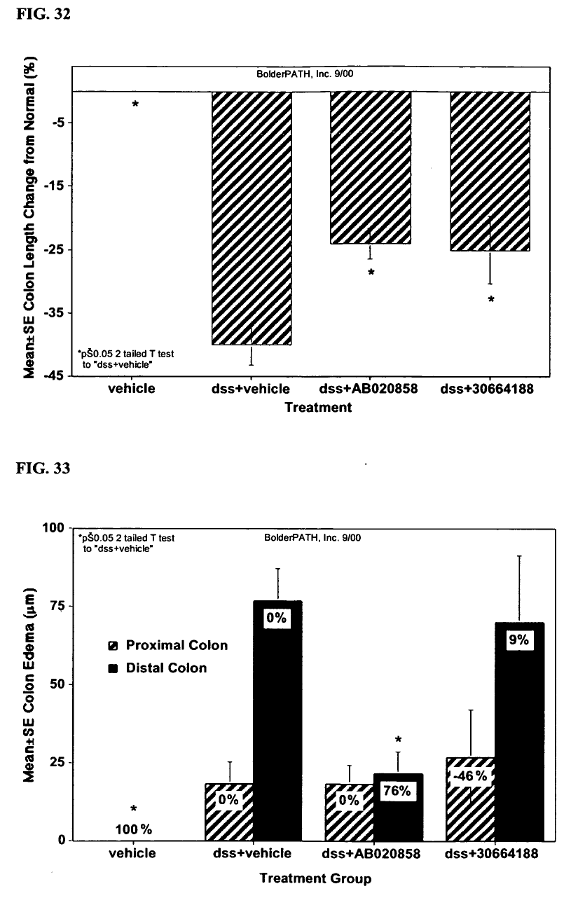

Absolute colon lengths (FIGS. 31 and 32) were decreased approximately 40% in mice treated with DSS+vehicle. Treatment with AB020858 (CG53135) resulted in significant (40%) reduction of the DSS-induced changes in colon length. Treatment with 30664188 (CG52053) reduced the colon length loss 36%.

Histopathology Findings: Histopathology was conducted on the full length of the colon. Lesions were much greater in the distal vs. proximal colon, as expected. Quantitation of efficacy of treatment is based primarily on inhibition of pathological changes in this location. Colonic edema in the distal colon was inhibited 76% by treatment with AB020858 (CG53135) whereas treatment with 30664188 (CG52053) did not inhibit the edema (FIG. 33).

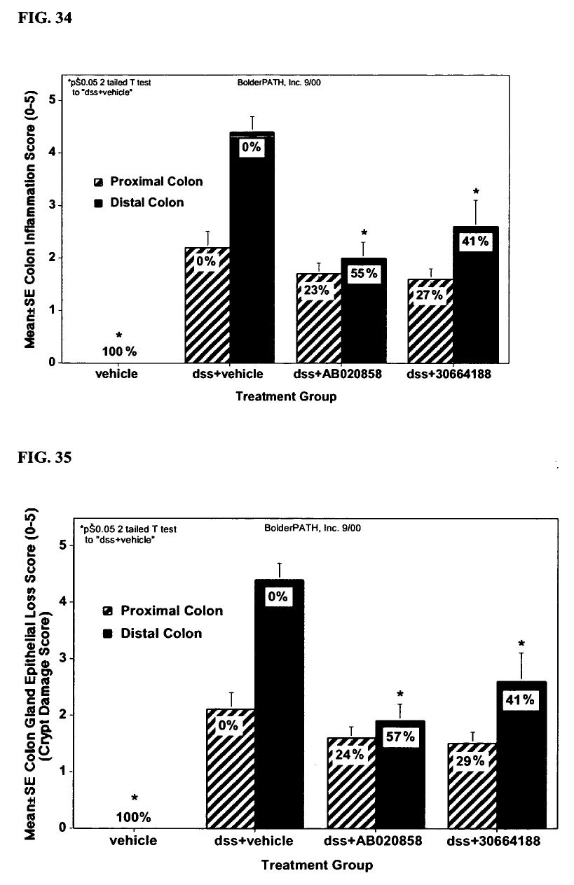

Colonic inflammation in the distal colon was inhibited 55% by treatment with AB020858 (CG53135) and 41% by treatment with 30664188 (CG52053) (FIG. 34).

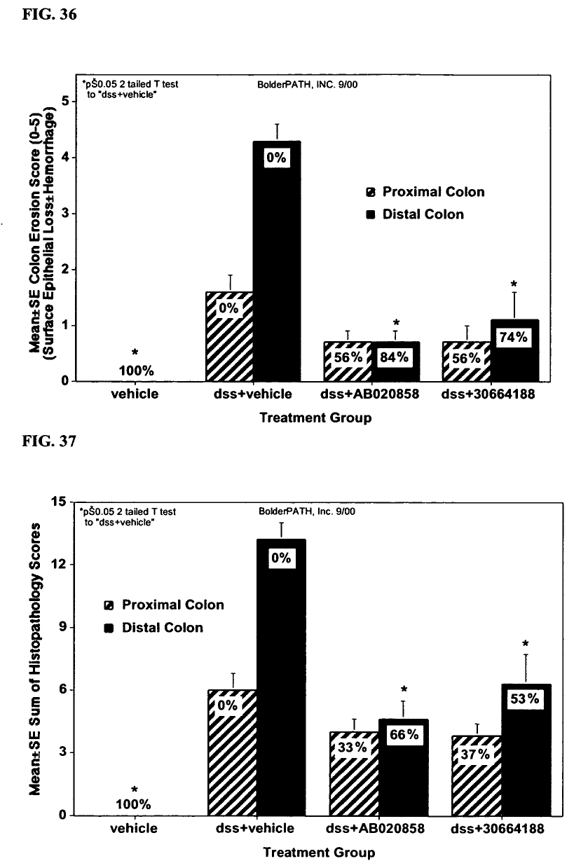

Protection of colonic epithelium (both crypts and remainder of the gland), as determined by the epithelial loss score, was 57% in mice given AB020858 (CG53135) and 41% in those treated with 30664188 (CG52053) (FIG. 35). Further evidence of mucosal epithelial protection in the distal colon was evident on evaluation of degree of surface epithelial loss leading to erosion/ulceration. As shown by the colon erosion scores, AB020858 (CG53135) treatment gave 84% inhibition of the erosive lesions and 30664188 (CG52053) treatment resulted in 74% inhibition (FIG. 36).

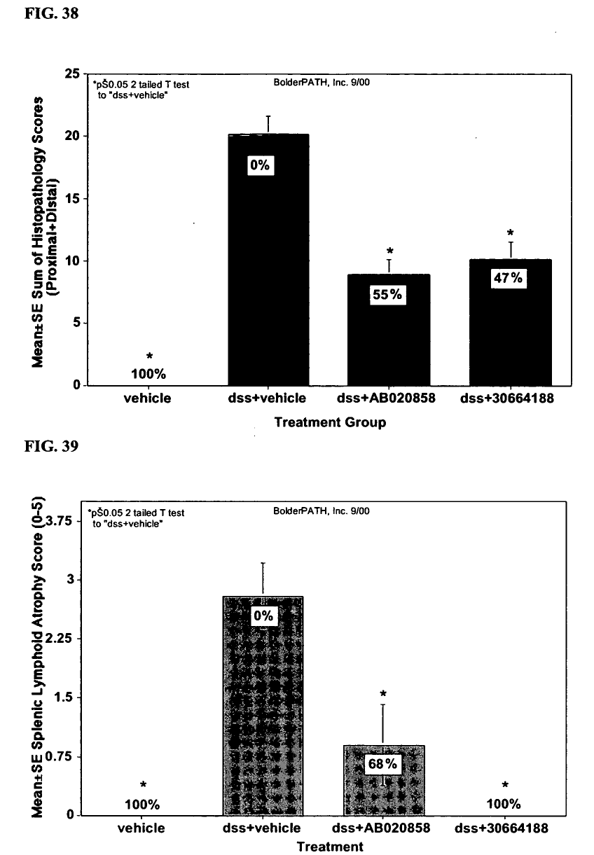

Summing the important histologic scores for inflammation, glandular epithelial damage and erosion (FIG. 37), it is seen that an overall protective effect results from the treatment with AB020858 (CG53135), which provides 66% inhibition of the pathology. Treatment with 30664188 (CG52053) resulted in 53% inhibition of the overall score. Slight but not significant (33-37%) inhibition of the total histologic scores was evident for proximal colon. Results for the colon overall are shown in FIG. 38.

Splenic weight decreases were largely a result of splenic lymphoid atrophy. Treatment with both proteins inhibited this parameter as well (FIG. 39).

Conclusion:

In this model of inflammatory bowel disease, in which mice are exposed to 5% DSS for 7 days, most animals develop marked to severe distal colonic inflammation/edema in association with crypt and colonic glandular epithelial loss and erosion/ulceration leading to marked hemorrhage. Lesions in the proximal colon are much milder but similar in character.

Cotemporaneous treatment with AB020858 (CG53135) (5 mg/kg, qd, d0-6) resulted in clinical benefit (reduced body weight loss) as well as protection against development of hemorrhagic diarrhea, a common feature of this model. Stressed unhealthy DSS treated mice have splenic lymphoid atrophy. This parameter (reflected by weight changes and histologic alterations) was also benefited by treatment with AB020858 (CG53135).

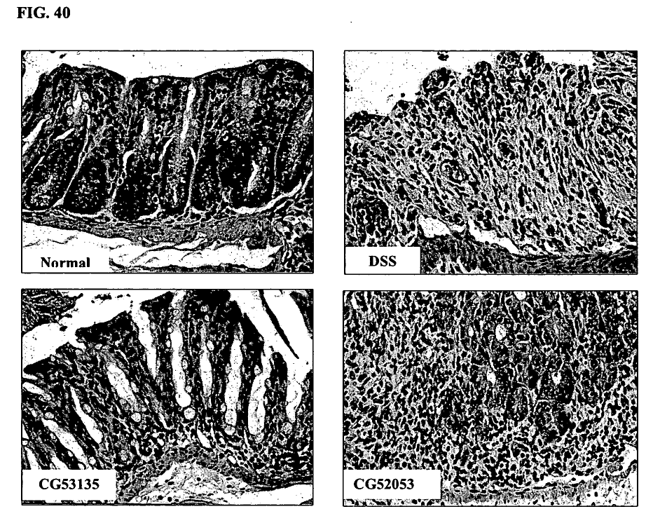

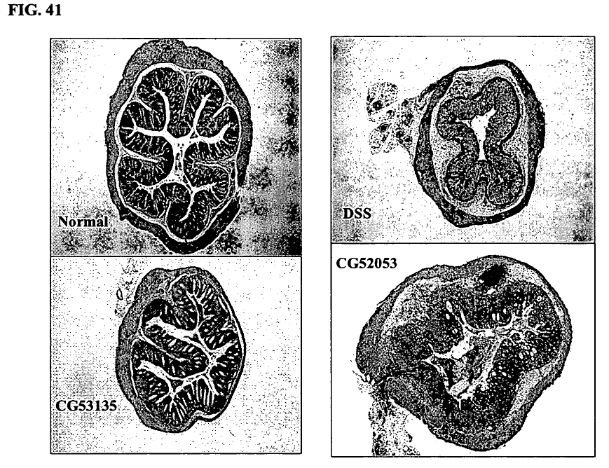

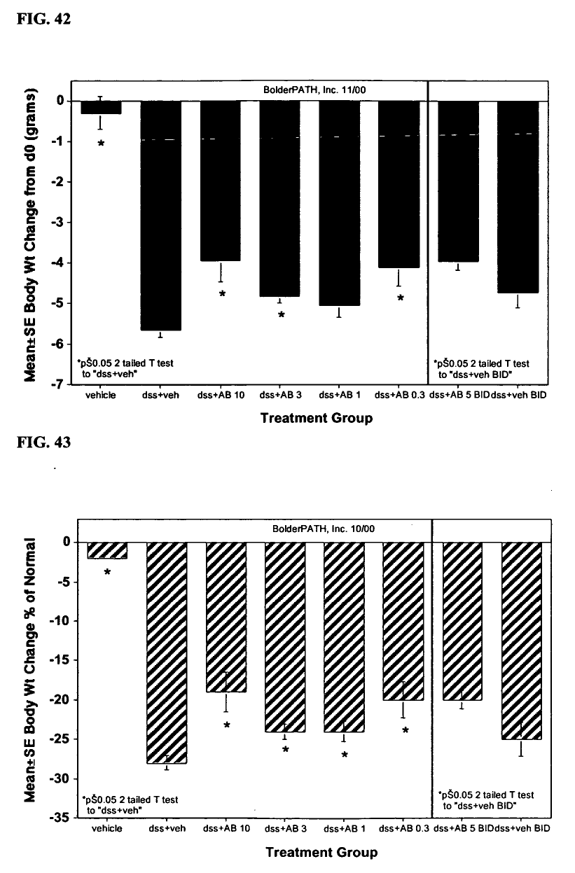

Colonic shortening (due to inflammation and mucosal tissue loss) was inhibited 40% by treatment with AB020858 (CG53135). This gross observation was strongly supported by the histologic observations of mucosal epithelial preservation in the crypts, colonic glands and surface epithelium (see FIGS. 40 and 41). In FIG. 40, viewed at 400× in the original images, the normal colonic mucosa has uniform glandular architecture and no submucosal edema (upper left). The disease control has no mucosal glands and surface epithelium, exposing blood vessels of the severely inflamed lamina propria to the lumen and resulting in hemorrhage (upper right). Treatment with CG53135 preserves mucosal integrity and results in decreased epithelial loss and reduced inflammation in the lamina propria (lower left). Treatment with CG52053 decreases epithelial loss and mucosal inflammation, although to a lesser degree than treatment with CG53135 (lower right). In FIG. 41, viewed at 50× in the original images, the normal control shows normal colonic mucosa with uniform glandular architecture and no submucosal edema (upper left). DSS-induced colitis results in loss of glandular architecture and edema that separates the mucosa from the outer muscle layers (upper right). Treatment with CG53135 inhibits the severe mucosal changes and submucosal edema induced by DSS (lower left). Treatment with CG52053 results in some inhibition of inflammation and loss of glandular architecture but no inhibition of submucosal edema (lower right). This histologic evidence of mucosal protection corroborates the dramatic necropsy observation that very little hemorrhagic diarrhea occurs.

The results of the experiments reported in this Example indicate that, in mice in which inflammatory bowel disease is induced by oral administration of DSS for 7 days, simultaneous treatment with the growth factors employed here during the course of exposure to DSS led to significant therapeutic benefits compared to untreated DSS controls.

### 6.12 Example 12

### Dose Responsive Effects of CG53135 in Female Swiss Webster Mice with Dextran Sulfate-Induced Colitis

The experiments reported in this Example report the results of dose titration experiments in an animal model of inflammatory bowel disease using a different strain of mouse than that used in Example 11.

Introduction and General Methods

Colitis Study Design: Normal female Swiss-Webster mice (Harlan Labs), 6-8 weeks old weighing approximately 20 g, were acclimated for 4 days (Day-4 through Day-1) and then given water orally (po) ad libitum containing 5% dextran sulfate sodium (DSS) or control water ad libitum for 7 days (Day 0 through Day 6). DSS (Spectrum Chemicals, Gardena Calif.) was made as a 5% solution in tap water; DSS was made every other day and stored at 4° C. Mice were divided into 8 treatment groups including QD doses of 0.3, 1, 3 and 10 mg/kg, and a BID dose regimen of 5 mg/kg per dose (Table 9). On Day 0, daily intraperitoneal (ip) treatments with vehicle (1M L-arginine in phosphate buffered saline) or CG53135 protein in vehicle were initiated and continued through Day 6. On Day 7, mice were sacrificed with CO₂.

TABLE 9
Treatment Groups

| | | Disease | | | | | Disease | |
|---|---|---|---|---|---|---|---|---|
| Treatment | Normal | Control^{b} | CG53135 | CG53135 | CG53135 | CG53135 | Control^{b} | CG53135 |
| Group | Control^{a} | QD | QD | QD | QD | QD | BID | BID |
| Group # | 1 | 2 | 3 | 4 | 5 | 6 | 7 | 8 |
| CG 53135 | 0 | 0 | 10 | 3 | 1 | 0.3 | 0 | 5 |
| (mg/kg) | | | | | | | | |
| Number | 4 | 10 | 10 | 10 | 10 | 10 | 10 | 10 |
| of Test | | | | | | | | |
| Animals | | | | | | | | |

^{a}normal control = vehicle only;
^{b}disease control = 5% DSS + vehicle

Protein production: The CG53135 protein was produced in E. coli as described in Example 11. The recombinant protein was purified by disrupting the E. coli cells (resuspended in a 1 M L-arginine solution) in a microfluidizer, followed by multiple metal affinity chromatography steps. The final purified protein was dialyzed into phosphate buffered saline containing 1M L-arginine.

Colon content was scored as described in Example 11.

Pathology Methods: Three sections equidistant apart from the distal one third of the colon (area that is most severely affected in this model) were processed for paraffin embedding, sectioned and stained with hematoxylin and eosin for pathologic evaluation.

For each section, scoring was done as described in Example 11. Splenic lymphoid atrophy was also scored by the above criteria. Epithelial cell loss/damage was scored as described in Example 11. Parameters that were scored using % involvement included: (1) Colon glandular epithelial loss—this includes crypt epithelial as well as remaining gland epithelial loss and would equate to crypt damage score; and (2) Colon Erosion—this reflects loss of surface epithelium and generally was associated with mucosal hemorrhage (reflective of the bleeding seen clinically and at necropsy). For each animal, 3 distal (most severe lesion) areas were scored. Scoring and analysis was done as described in Example 11.

Live Phase, Necropsy and Organ Weight Results

Four animals died during the course of the study (#10 in vehicle control group 2 on day 7, #3 in group 6, 0.3 mg/kg on day 6, #5 in group 8 vehicle control BID on day 7, and #6 in group 7 5 mg/kg BID on day 6).

DSS treatment-related changes in body weight were obvious by day 5 in all DSS treated mice and ultimately were most severe in animals treated with vehicle (FIG. 42). At study termination, DSS+vehicle controls had a 28% decrease in body weight. A significant beneficial effect on DSS induced weight loss was seen in mice given AB020858 (CG53135) QD at all doses (FIG. 43).

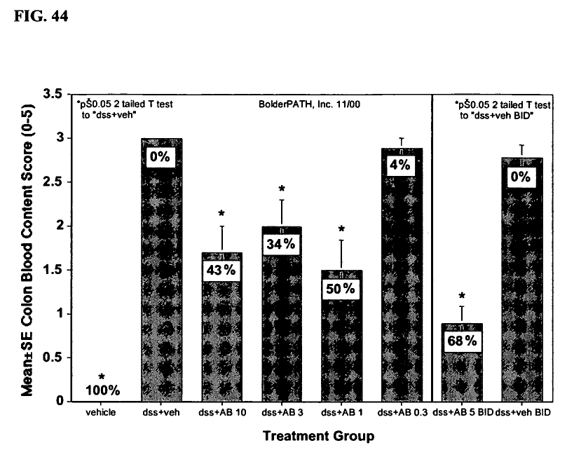

Clinical evidence of bloody diarrhea was evident in all DSS+vehicle animals. At necropsy all DSS controls had blood or blood tinged fluid in the colon. In contrast, mice treated QD with 10 mg/kg AB020858 (CG53135) generally had semi-solid stool and less blood (except animals #5). Clinical benefit was also evident but less impressive in those given doses of 3 or 1 mg/kg QD and absent in those treated with 0.3 mg/kg (FIG. 44). Mice treated BID with 5 mg/kg had the most impressive clinical benefit (68% inhibition) and clinically these mice had the best overall improvement.

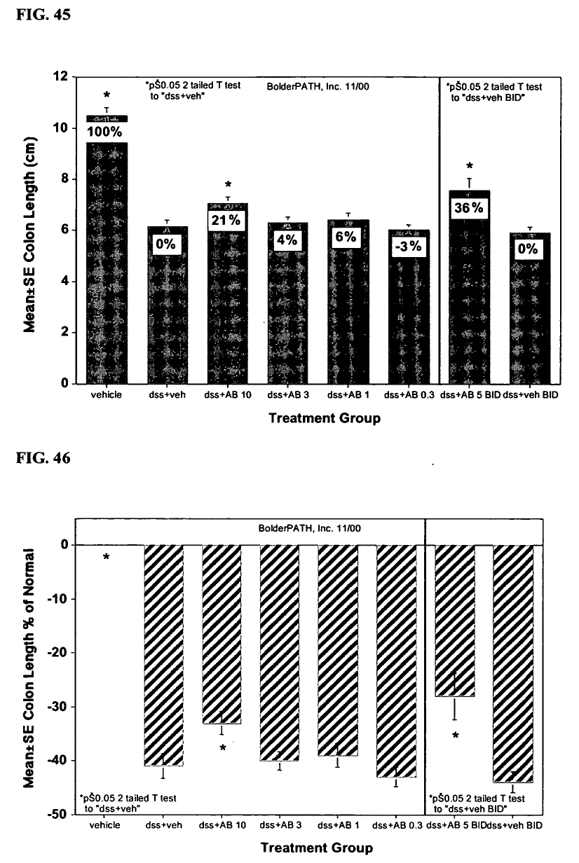

Absolute colon lengths (FIGS. 45 and 46) were decreased 41% in mice treated with vehicle. Treatment with AB020858 (CG53135) QD at 10 mg/kg resulted in significant (21%) inhibition of the DSS-induced changes in colon length. Treatment with AB020858 (CG53135) BID at 5 mg/kg reduced the colon length decrease 36%.

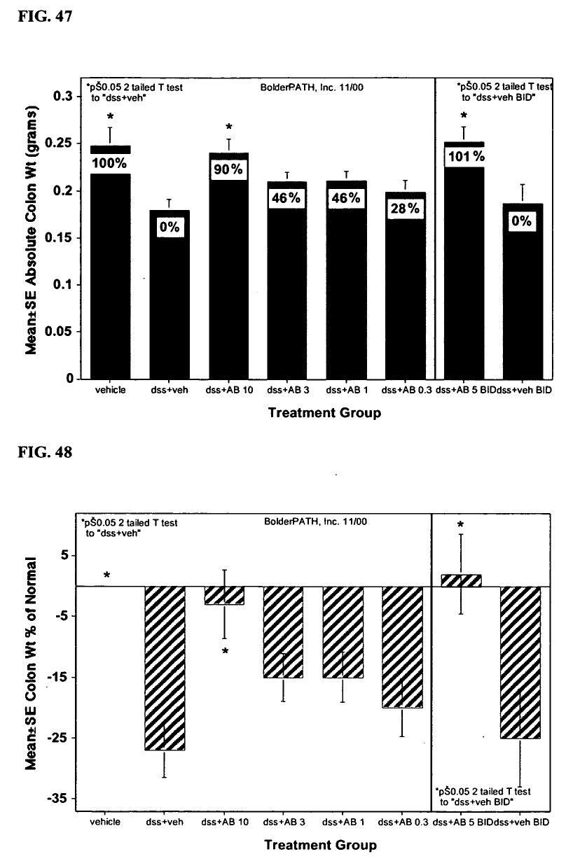

Absolute colon weights (FIGS. 47 and 48) were decreased approximately 26% in mice treated with DSS in vehicle. Treatment with AB020858 (CG53135) at 10 mg/kg QD or 5 mg/kg BID resulted in significant reduction of the DSS-induced changes in colon weights.

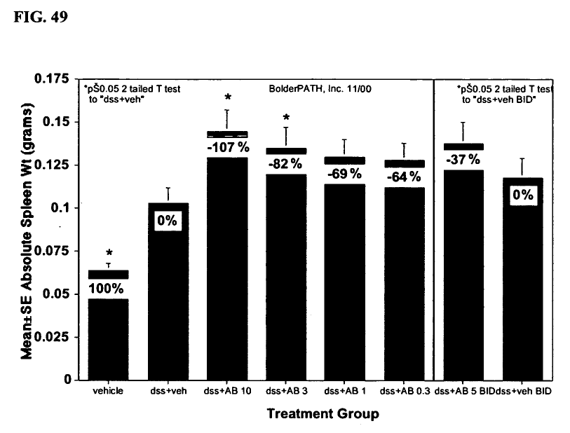

Absolute spleen weights (FIG. 49) were increased approximately 40% in mice treated with DSS+vehicle (due to extreme extramedullary hematopoiesis). Spleen weights were significantly greater in all DSS treated animals vs. normal.

Histopathology Findings

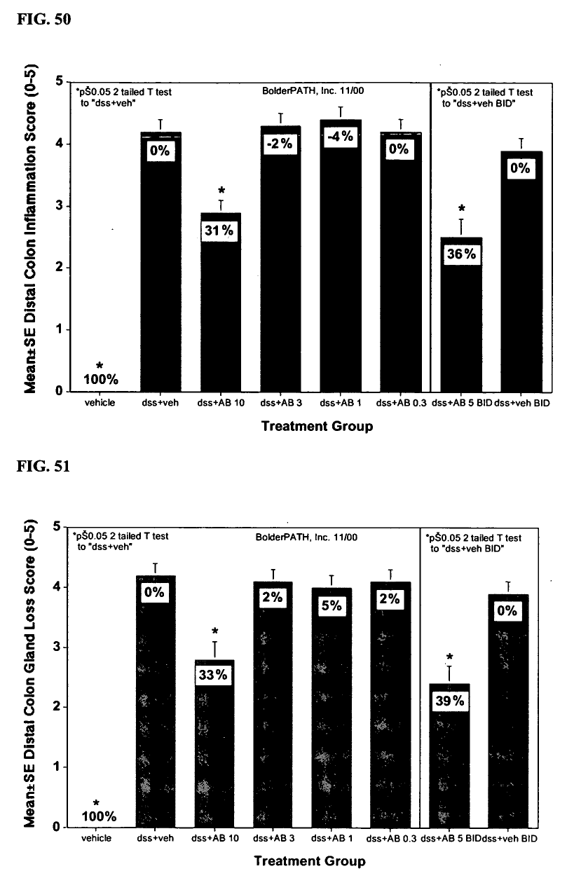

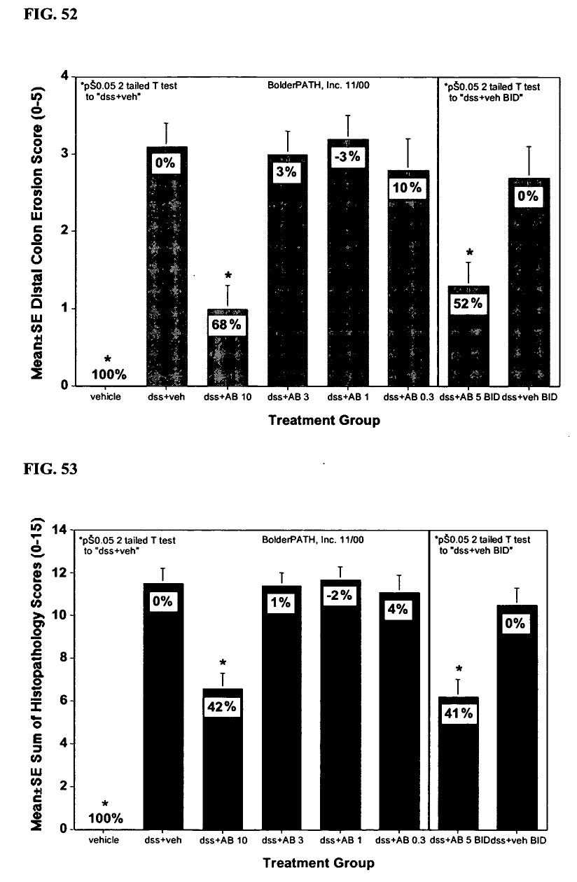

Significant reduction of colonic inflammation, gland loss, erosion and total histopathology scores occurred in mice treated with AB020858 (CG53135) QD (10 mg/kg) and BID (5 mg/kg) and was of approximately equal magnitude (FIGS. 50, 51, 52 and 53).

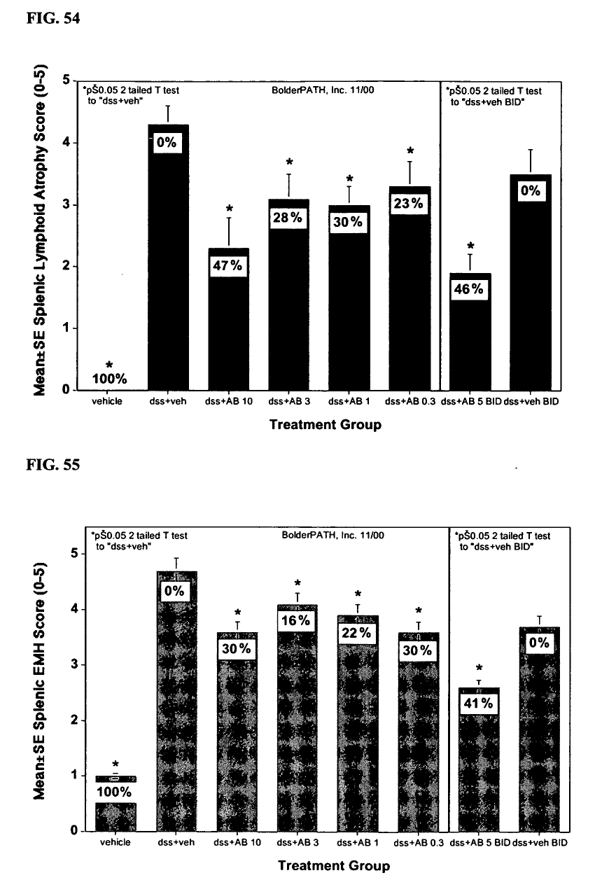

Splenic lymphoid atrophy (an indication of stress) was inhibited in these same animals 47% and 46% respectively (FIG. 54). Inhibition of induction of splenic extramedullary hematopoiesis was greater in mice treated BID vs. QD and occurred in all treatment groups (FIG. 55).

Discussion and Conclusions

The experiments reported in this Example provide dose-response information for the administration of AB020258 (CG53135), using a different strain of mouse than those in Example 11 (which used Balb/c mice). The results indicate that simultaneous administration of AB020258 (CG53135) is effective in inhibiting the appearance of markers of DSS-induced inflammatory bowel disease, especially with the highest doses used.

An additional experiment was carried out in which mice were also treated subcutaneously with CG53135. Together with the results in Examples 11 and 12, these studies demonstrate that prophylactic administration of CG53135 at doses of 5 or 10 mg/kg ip and 5 or 1 mg/kg sc significantly reduce the extent and severity of mucosal damage induced by dextran sulfate sodium in a murine model of colitis.

### 6.13 Example 13

### Effects of Administering CG53135 to Indomethacin-Treated Rats

Treatment of rats with indomethacin results in gross and histopathologic intestinal alterations that are similar to those occurring in Crohn's Disease. The experiments provided in this Example report on the efficacy of CG53135 in treating the rat model of indomethacin-induced intestinal injury. The efficacy of this protein in an alternate model of intestinal injury adds support to the therapeutic potential of CG53135 in treatment of inflammatory bowel disease.

Materials & Methods

Protein production: Preparation of CG53135 protein was the same as described in Example 11.

Study Design: Female Lewis rats (Harlan, Indianapolis, Ind.) weighing 175-200 g were acclimated for 8 days (Day-8 through Day-1). Rats were divided into 8 treatment groups: four groups receiving CG53135 (three groups iv and one group sc), two iv controls for normal and the disease model, and two sc controls for normal and the disease model. On Day-1, treatments with CG53135 or vehicle were initiated and continued through Day 4. CG53135 was injected iv (tail vein) at doses of 5, 1 or 0.2 mg/kg, or 1 mg/kg sc; vehicle controls were injected with BSA (5 mg/mL in PBS+1M L-arginine). On Days 0 and 1 rats were treated with indomethacin (Sigma Chemical Co., St. Louis, Mo.; 7.5 mg/kg doses) in order to induce gross and histopathologic intestinal alterations similar to those occurring in Crohn's Disease. Indomethacin was prepared in 5% sodium bicarbonate. On Day 5, rats were injected with a single ip dose of 50 mg/kg 5-bromo-2′deoxyuridine (BrdU, Calbiochem, LaJolla, Calif.) 1 hour prior to necropsy in order to pulse label proliferating cells in the intestine and spleen. Following termination, a 10 cm section of jejunum in the area at risk for lesions was weighed, given a gross pathology score, and then collected into formalin for histopathologic evaluation and scoring of necrosis and inflammation. Blood was collected for CBC analysis.

Observations and Analysis of Markers of Pathology

Gross Observations: Body weight was measured daily beginning on Day 0. At necropsy, liver and spleen weights were measured, and a 10 cm section of jejunum in the area at risk was weighed, scored for gross pathology, and collected into formalin for histopathologic evaluation and scoring of necrosis and inflammation. The area at risk for indomethacin-induced injury was scored at necropsy according to the following criteria: 0=normal; 1=minimal thickening of the mesentery/mesenteric border of the intestine; 2=mild to moderate thickening of the mesentery/mesenteric border of intestine, but no adhesions; 3=moderate thickening with 1 or more definite adhesions that are easily separated; 4=marked thickening with 1 to numerous hard to separate adhesions; and 5=severe intestinal lesions resulting in death.

Histopathology: Five sections (approximately equally spaced) taken from the weighed 10 cm area at risk of small intestine for indomethacin-induced lesions were fixed in 10% neutral buffered formalin, processed for paraffin embedding, sectioned at 5 μm and stained with hematoxylin and eosin for histopathologic evaluation. Necrosis was scored according to the percent area of the section affected in the same way as described in Example 11 for scoring epithelial cell loss.

Inflammation was scored according to the following criteria: 0=none; 1=minimal inflammation in mesentery and muscle or lesion; 2=mild inflammation in mesentery and muscle or lesion; 3=moderate inflammation in mesentery and muscle or lesion; 4=marked inflammation in lesion; and 5=severe inflammation in lesion.

The means for inflammation and necrosis were determined for each animal, and then the means for each group were calculated.

Statistics. The mean and standard error (SE) for each treatment group were determined for each parameter scored; the data were compared to the data for the disease controls using a 2-tailed Student's T test with significance at p<0.05.

Results

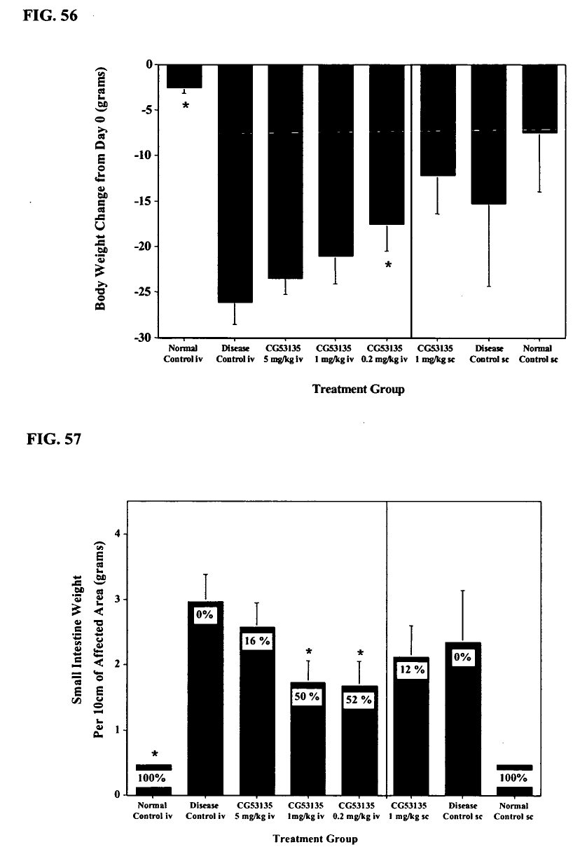

Weight loss was observed in all animals treated with indomethacin. A slight, but significant reduction in weight loss was observed in animals treated with CG53135 (0.2 mg/kg iv) as compared with disease controls (iv). Other doses of CG53135 (both iv and sc routes of administration) provided diminished, but not statistically significant, indomethacin-induced weight loss (FIG. 56).

At necropsy, a 10 cm section of jejunum in the area at risk from each animal was weighed. Indomethacin treatment resulted in an elevation in small intestine weight as compared with normal iv and sc controls, consistent with edema and inflammation associated with this model of intestinal injury. Treatment with CG53135 (1 mg/kg or 0.2 mg/kg iv) resulted in significant reductions in small intestine weight as compared with disease controls (FIG. 57). A slight reduction in the small intestine clinical score was observed, with the greatest benefit occurring with the 1.0 mg/kg iv dose (38%) and the 0.2 mg/kg iv dose (25%); these benefits, however, were not statistically significant. Relative spleen and liver weights were increased in animals treated with indomethacin. Administration of CG53135 produced moderate additional increases in these weights.

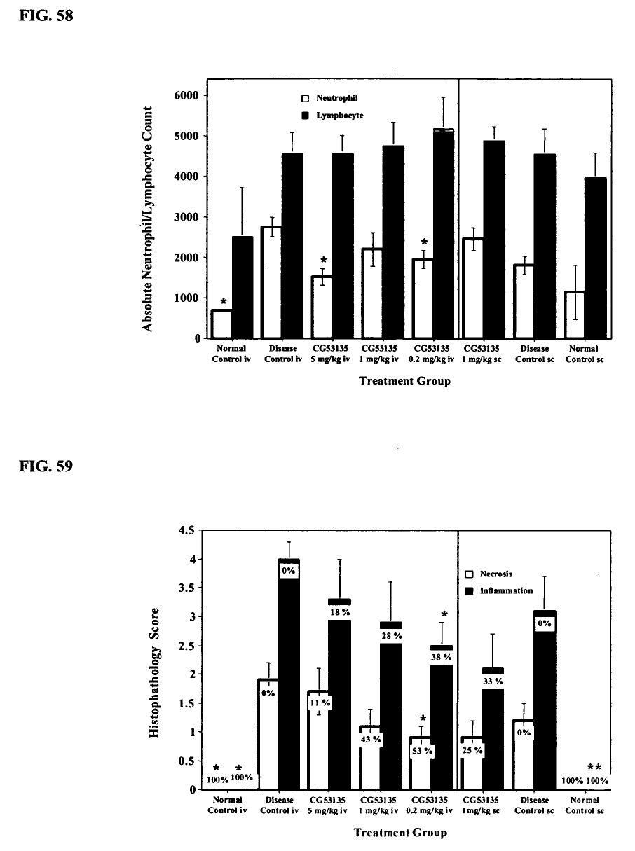

Hematology: Administration of 2 doses of indomethacin to rats increased the total white blood cell count as a result of increased neutrophils and lymphocytes. Reductions in red blood cell count, hematocrit, and hemoglobin concentration were also observed. Treatment with CG53135 (5 mg/kg and 0.2 mg/kg iv) resulted in significant reductions in absolute neutrophil counts as compared with disease controls (FIG. 58). Hemoglobin concentration was diminished in the indomethacin controls compared to normal controls, and slightly further diminished in rates treated with CG53135.

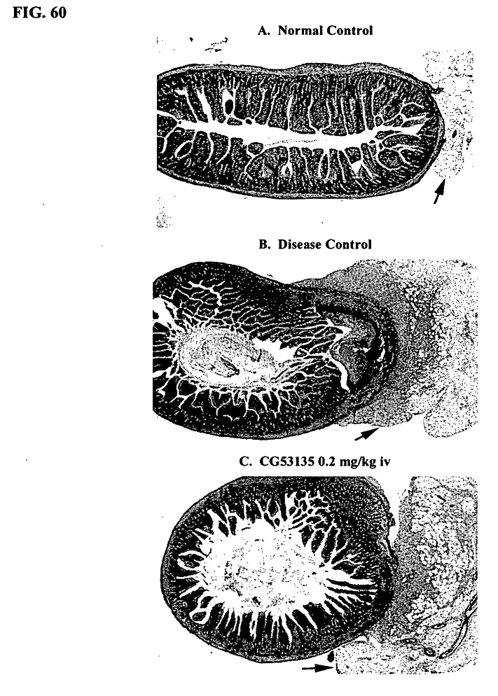

Histopathology: Evaluation and scoring of 5 sections of intestine were conducted for each animal. Histologic evidence of a protective effect on the intestine was observed only in animals treated with CG53135 (0.2 mg/kg iv). A 53% reduction in jejunal necrosis and 38% reduction in inflammation score were observed for the 0.2 mg/kg iv CG53135 dose as compared with disease controls iv (FIG. 59). Photomicrographs of affected small intestine are shown in FIG. 60 for a normal and disease control, and a rat treated with 0.2 mg/kg CG53135. Panel A shows the small intestine from a normal control animal treated iv with vehicle (BSA). Normal villous architecture and mesentery (arrow) are apparent. Panel B presents a photomicrograph of the small intestine from an indomethacin-treated rat, with vehicle (BSA) iv. Focal mucosal necrosis extending across most of the area associated with attachment of the mesentery is apparent (see, for example, the asterisks at upper right intestinal wall and lower right intestinal wall). Marked inflammatory cell infiltrate is present in the mesentery (arrow). Panel C shows the image of the small intestine from an indomethacin-treated rat further treated with CG53135, 0.2 mg/kg iv. There is no apparent necrosis, in contrast to the disease control shown in Panel B. There is a focal area of attenuated villi and cellular infiltration into muscle layer (see, for example, the three asterisks at the upper right, right and lower right of the intestinal wall). Mesentery (arrow) is infiltrated by inflammatory cells. The photomicrographs in FIG. 60 provide further support for the protective effect of 0.2 mg/kg iv CG53135.

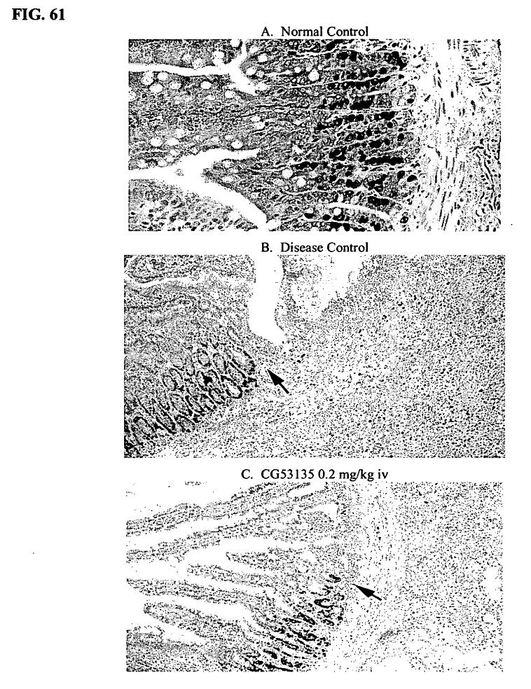

BrdU labeling was carried out by injecting 50 mg/kg 1 hour prior to necropsy. In the small intestine from a normal control animal, normal pattern of crypt labeling is seen at 100× (FIG. 61, Panel A). BrdU incorporation in the disease model was decreased or absent in epithelial cells in mucosal areas of necrosis, but increased in subajacent inflammatory tissue in which fibroblast labeling was prominent (FIG. 61, Panel B, visualized at 50×). Focal mucosal necrosis (arrow) is delineated by an absence of BrdU immunostaining as well as severe infiltration of inflammatory cells and fibroblast proliferation. Small intestine from a rat treated with indomethacin+CG53135 0.2 mg/kg iv shows an absence of crypt labeling, but relatively intact mucosa (arrow in FIG. 61, Panel C, visualized at 50×). Subadjacent smooth muscle and mesentery is only mildly infiltrated with inflammatory cells, compared with that seen in the disease control (Panel B). In certain animals treated with CG53135, in which preservation of mucosal integrity occurred, increased crypt labeling was also observed; this is in the direction found in the normal control.

The results of the experiments in this Example may be summarized as follows: treatment of rats with indomethacin results in gross and histopathologic intestinal alterations that are similar to those occurring in Crohn's Disease. Administration of CG53135 (0.2 mg/kg iv) to indomethacin-treated rats resulted in significant reductions in weight loss, small intestine weight, absolute neutrophil counts, and jejunal necrosis and inflammation scores. Higher doses of CG53135 (5, 1 mg/kg iv and 1 mg/kg sc) were less efficacious. The morphological appearance of tissues collected from animals injected with BrdU 1 hour prior to necropsy suggested that the beneficial effects of CG53135 in this model of intestinal injury were the result of mucosal protection rather than a proliferative effect on target cells.

### 6.14 Example 14

### Therapeutic Administration of CG53135 Enhances Survival in the Murine DSS Model

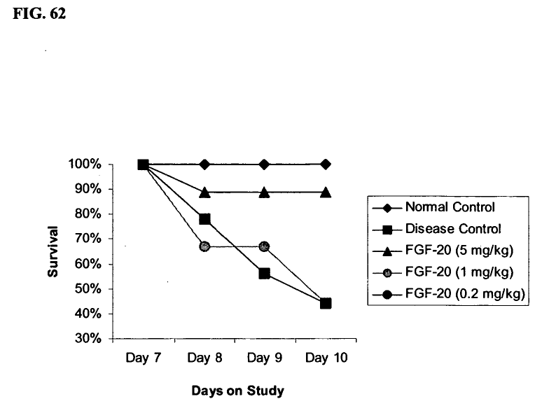

In the experiments described in Examples 11-13, DSS exposure and CG53135 administration were initiated simultaneously on day 0. In the present Example, the effect of CG53135 administered after the initiation of DSS treatment was examined. CG53135 was prepared as described in Example 11. Balb/c mice were exposed to DSS for 7 days (day 0 to day 6). The mice were injected daily subcutaneously with various concentrations of CG53135 (5, 1 and 0.2 mg/kg) beginning on the fifth day of DSS exposure (i.e. day 4) and ending 3 days after the termination of DSS exposure (i.e. day 9), or with vehicle only. Animal survival was recorded on a daily basis and the experiment was concluded on day 10. As shown in FIG. 62, therapeutic administration of CG53135 at 5 mg/kg enhanced survival relative to the disease control group. Thus, while only 44% (4 of 9) of the animals in the disease control group survived until the end of the study, 89% (8 of 9) of the animals treated with CG53135 at 5 mg/kg survived.

### 6.15 Example 15

### Effects of CG53135 in an Immune-Mediated Model of Inflammatory Bowel Disease in IL-10 Deficient Mice (IL-10 Knock-Out Mice)

The objective of the study was to assess the ability of CG53135 to therapeutically inhibit the inflammation that occurs in IL-10 deficient mice when transferred from a germ free to a specific pathogen free environment. As inflammatory bowel disease is thought to have an immune component, this study evaluated the efficacy and safety of CG53135 in this immune-mediated model of IBD when dosed therapeutically at the time of significant disease.

TABLE 10
Materials and Methods

| | |
|---|---|
| Species/strain: | IL-10 Knock-out Mice (mixed C57BL X 129 Ola |
| | background) |
| Physiological state: | Germ-free |
| Age/weight range | ˜8-12 weeks old weighing approximately |
| at start of study: | 20-25 g |

Test Article: CG53135-05 (FGF-20) protein (purity>97%) in 20% glycerol buffer.

Storage Conditions of test article: All tubes were stored at −70° C. until ready for use.

Vehicle: Glycerol buffer: 20% Glycerol, 200 mM Sorbitol, 1 mM EDTA, 100 mM Citrate, 50 mM KCL Storage Conditions of vehicle: All tubes were stored at −70° C. until ready for use.

TABLE 11
Administration of Test Article

| | |
|---|---|
| Route and method of | Intraperitoneal (ip) |
| administration: | |
| Justification for route | This route and dose has been used in |
| of administration: | previous studies with CG53135 in other |
| | murine models of colitis. |
| Frequency and duration | Once daily |
| of dosing: | |
| Administered doses of | 5.0 mg/kg in 20% glycerol buffer |
| CG53135-05: | |
| Administered volume: | 0.3 mL per mouse |
| Justification for dose | Similar doses have been used in other |
| levels: | efficacy models. |

TABLE 12
Experimental Study Design

| Group | | Number of Animals | | Dose | Volume |
|---|---|---|---|---|---|
| Number | Treatment | Females | Males | (mg/kg) | (mL/kg) |
| 1 | Normal control^{a} | 4 | 4 | 0 | 10 |
| 2 | Disease control^{b} | 4 | 4 | 0 | 10 |
| 3 | 5 mg/kg CG53135 | 4 | 4 | 5 | 10 |
| | (14 d therapeutic) | | | mg/kg | |

^{a}Normal control: mice are untreated and maintained in germ free conditions throughout study
^{b}Disease control: vehicle administered ip, once daily using the therapeutic dosing regimen.

TABLE 13
Study Schedule
Study Schedule

| | Day of Study | | | | | | | | | | | | | | | | | | | | | | | |
|---|---|---|---|---|---|---|---|---|---|---|---|---|---|---|---|---|---|---|---|---|---|---|---|---|
| Event | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
| Transfer from | X | | | | | | | | | | | | | | | | | | | | | | | |
| germ free cages | | | | | | | | | | | | | | | | | | | | | | | | |
| Fecal slurry^{a}, po | | | X | | | | | | | | | | | | | | | | | | | | | |
| Therapeutic | | | | | | | | | | X | X | X | X | X | X | X | X | X | | | | | | |
| CG53135 (ip) | | | | | | | | | | | | | | | | | | | | | | | | |
| Therapeutic | | | | | | | | | | X | X | X | X | X | X | X | X | X | | | | | | |
| Vehicle (ip) | | | | | | | | | | | | | | | | | | | | | | | | |
| Body weight | X | X | X | X | X | X | X | X | X | X | X | X | X | X | X | X | X | X | | | | | | |
| Serum collected | | | | | | | | | | X | | | | | | | | | X | | | | | |
| for CG53135 | | | | | | | | | | | | | | | | | | | | | | | | |
| antibodies | | | | | | | | | | | | | | | | | | | | | | | | |
| Tissues collected | | | | | | | | | | X | | | | | | | | | X | | | | | |
| (including | | | | | | | | | | | | | | | | | | | | | | | | |
| colon assessments) | | | | | | | | | | | | | | | | | | | | | | | | |
| Scheduled | | | | | | | | | | X | | | | | | | | | X | | | | | |
| terminations | | | | | | | | | | | | | | | | | | | | | | | | |

^{a} Mice are dosed orally with a slurry of fecal contents solubilized in PBS from donor SPF documented free of Heliobacter.
^{b} CG53135-05 or vehicle will be administered daily through to day prior to scheduled termination.

### EXPERIMENTAL PROCEDURES

Mice were acclimated for 2 days before bacterial colonization and given autoclaved food and tap water ad libitum during this time. Mice will be treated with CG53135-05 E. coli purified product or buffer for 2 days beginning the day of transfer, then colonized with specific pathogen free bacteria by swabbing their mouth and rectum with solubilized fecal material. Animals were examined prior to initiation of the study to assure adequate health and suitability. Animals that were found to be diseased or unsuitable will not be assigned to the study.

This study was performed in two segments of approximately 20 animals each due to animal availability and the tedious collections of cells and tissues at necropsy for T cell stimulation and colonic strip culture. The two study segments had mice evenly assigned to all dose groups (e.g., 2 animals per sex per treatment group). If the number of available animals at the time of initiation is not evenly divided between males and females, animals were assigned to groups to balance males and females as best as possible.

Clinical Observations/Signs

Mice were observed daily for significant clinical signs of toxicity, moribundity and mortality approximately 60 minutes after dosing.

### 6.16 Example 16

### Effects of CG53135 on Body Weight, Histopathology, and Cytokine Releases

Body Weight: Individual body weights of all mice was recorded pretest (for randomization) and daily through Day 10. Body weights taken on the day of necropsy for animals scheduled for termination was used for determination of organ to body weight ratios. Following are the organs/tissues measured.

TABLE 14
Organs/Tissues For Weight Measurement

| | | |
|---|---|---|
| Cecum | Kidneys | Rectum |
| Colon | Liver | Spleen |

Histopathology: All animals surviving to scheduled termination (Day 10) will be terminated using CO₂ with assessment of gross observations, organ weights and collection of all scheduled tissues into 10% neutral buffered formalin for histopathologic evaluation.

Special colon assessments: From the areas at risk (cecal tip, transverse colon and rectum), 3 sections approximately 1 cm apart in length will be collected, preserved in formalin, and stained to quantitate inflammation (hematoxylin & eosin), mucin (periodic acid schiff), and collagen (trichrome). All 3 sections should be representative of the affected area.

Tissues taken from the colon, will be collected and processed for paraffin embedding, sectioned and stained as noted above. Histopathology will be performed in a blinded manner on the tissue samples of cecum, transverse colon and rectum with assignment of an inflammation score ranging from 0 to 4, where: 0=no inflammation; 1=mild inflammation with increased mononuclear cells infiltrating, mild crypt hyperplasia; 2=more active inflammation with increased infiltrating mononuclear cells, mild goblet cell depletion, and mild crypt hyperplasia; 3=active inflammation with crypt hyperplasia, goblet cell depletion and marked mononuclear cell infiltration; and 4=severe active inflammation characterized by widespread infiltrate of neutrophils, ulceration, crypt abscesses and marked mucosal hyperplasia.

Following were the organs/tissues considered for macroscopic examination and histopathology.

TABLE 15
Organs/Tissues For Histopathology Evaluation*

| | | |
|---|---|---|
| Cecum | Eyes | Rectum |
| Colon | Kidneys | Spleen |
| | Liver | |

*All tissues except eyes to be fixed in 10% neutral buffered formalin, eyes to be fixed in 6% glutaraldehyde.

Preparation of cells and cell cultures: Colonic strip cultures were established from the remaining colon fragments, pooled from segments of proximal, middle and distal colon. Colon segments were flushed with phosphate-buffered saline (PBS) to remove fecal contents, opened lengthwise and cut into 0.5 to 1.0 cm pieces and shaken vigorously in PBS. Approximately 50 to 100 mg of tissue was then distributed per well of a 24 well tissue culture plate in duplicate and cultured in 1 mL of complete medium containing antibiotics and an antimycotic agent (Veltkamp et al, 2001, Gastroenterology, Vol 120 (4): 900-913). After incubation at 37° C. for 18-24 hours, culture supernatants were collected in aliquots and frozen at −70° C. for cytokines and possibly immunoglobulin measurements. IgG2a and IL-12 in the supernatant were measured by ELISA.

Mesenteric lymph nodes (MLN) was mechanically dispersed, washed, counted and used for cecal bacterial lysate-stimulated interferon gamma measurement as described by Veltkamp et al, 2001, Gastroenterology, Vol 120 (4): 900-913. Briefly, CD4+T lymphocytes were isolated by negative MACS selection and incubated with antigen-pulsed antigen presenting cells derived from wild type mice splenocytes after T cell removal. Alternatively, unfractionated MLN cells were incubated with antigen.

Cytokine assays: IL-12 (Pharmingen, San Diego, Calif.), TNF-a and IFN-? (R&D systems, Minneapolis, Minn.) was measured in MNL cell and splenocyte culture supernatants by ELISA. Moreover, IL-12 and PGE2 (Assay Design, Ann Arbor, Mich.) was measured in supernatants of colon cultures using standard ELISA protocol. Concentrations of these cytokines and PGE2 were measured in duplicate culture supernatants by comparison with standard curves generated using recombinant cytokines.

Results

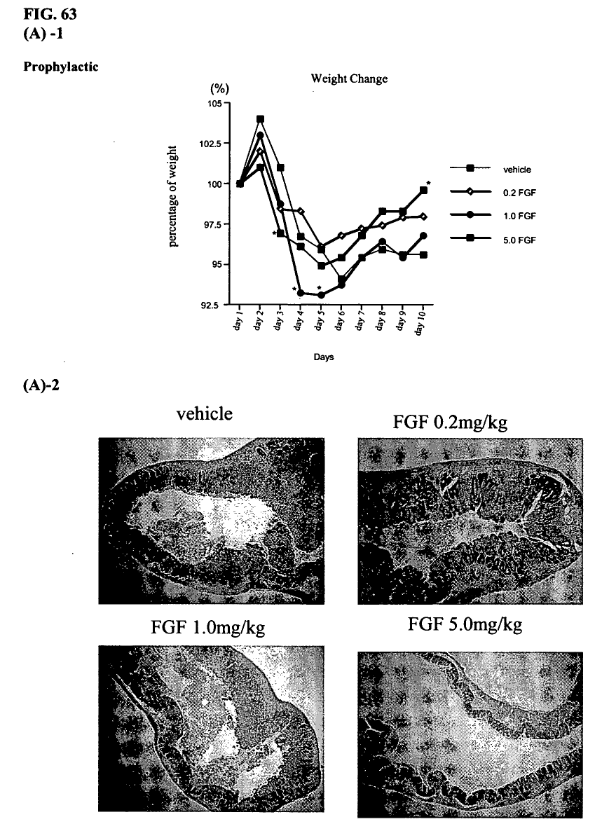

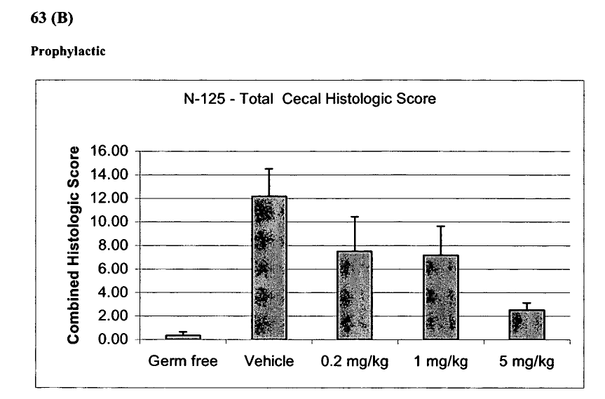

Body Weight and Histopathology—Prophylactic: Weight change in the prophylactic group was assessed. FIG. 63(A) shows weight change when challenged with FGF-20 in IL-10 knock-out (KO). FIG. 63(A) also shows the histopathology of the colon when the mice are challenged with different concentrations of FGF-20 (0.2, 1, 5 mg/kg). The results indicate that, administration of FGF-20 had a protective effect as compared to the vehicle control. FIG. 63(B) further demonstrates that, upon administration of FGF-20, there is a dose-dependent decrease in the total Cecal Histologic score, as compared to the vehicle (12.2±2.3 vs. 2.5±0.6; p<0.001).

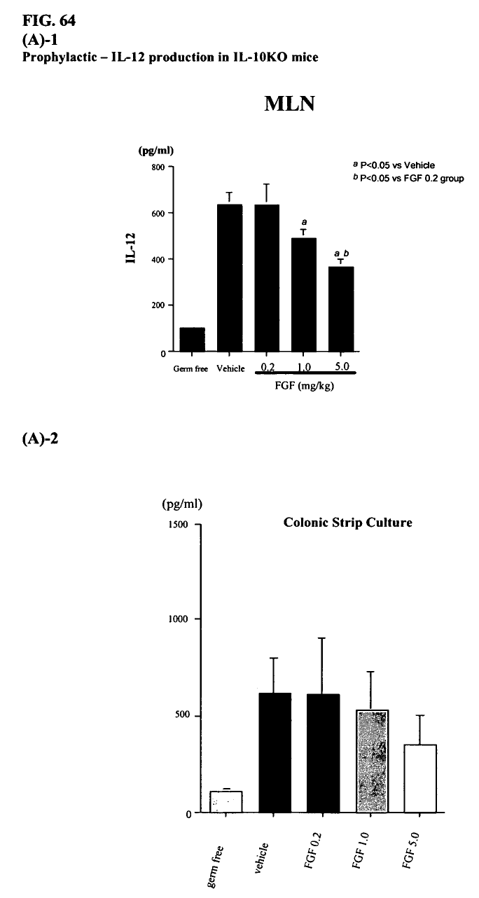

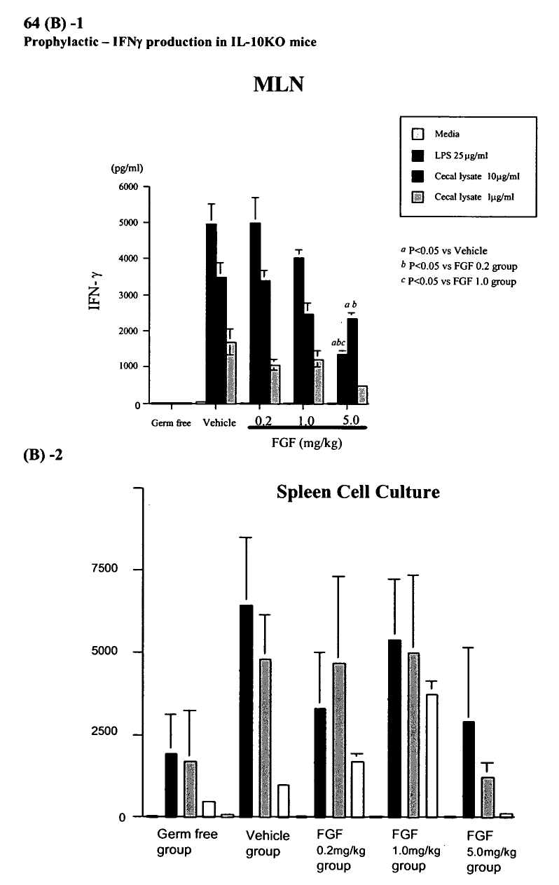

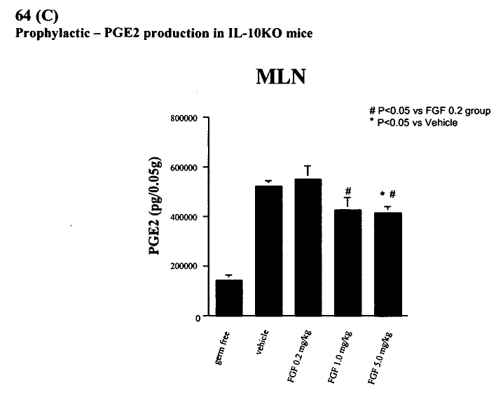

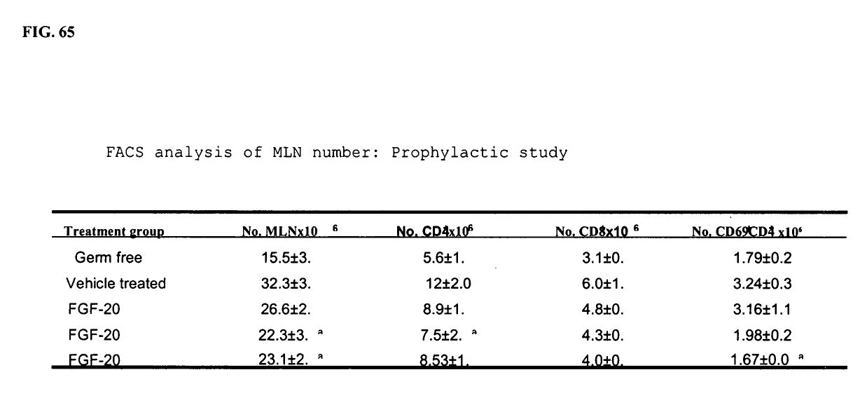

Cytokine Production—Prophylactic: Cytokine production was assayed by ELISA. FIGS. 64(A) (IL-12), 64(B) (IFN?) and 64(C) (PGE2) indicate that FGF-20 altered cytokine production in MLN, colonic strip culture, Spleen cell culture, which were prepared from the IL-10KO mice as described in Example 15. FIG. 65 also shows FACS analysis of total MLN number (32±3.4 vs. 23±2.5; p<0.05), CD4+ and CD8+ and CD4+ CD69+ cells (3.2±0.3 vs. 1.67±0.1; p<0.05).

Body Weight and Histopathology—Treatment: Study protocol for the treatment group was established by treatment of established colitis in ex-germ free IL-10−/− mice colonized with SPF bacteria on day 1. On day 10, treatment was started by intraperitoneally administering either Vehicle or FGF-20 (5 mg/kg) and necropsy was performed on day 17.

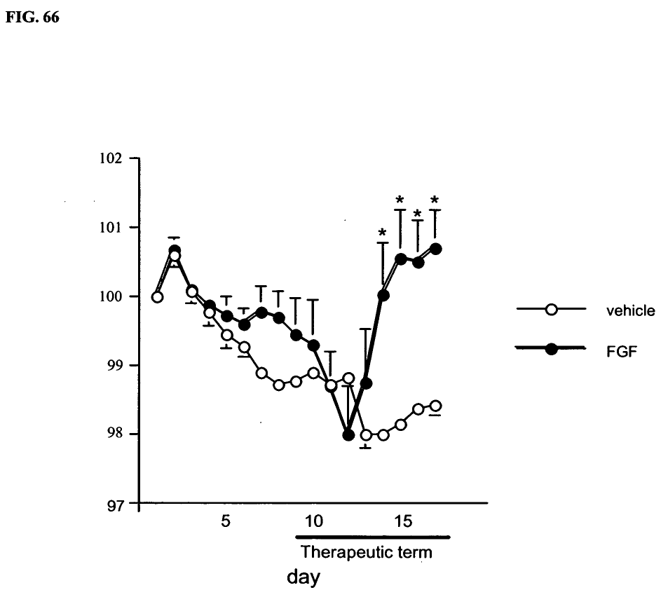

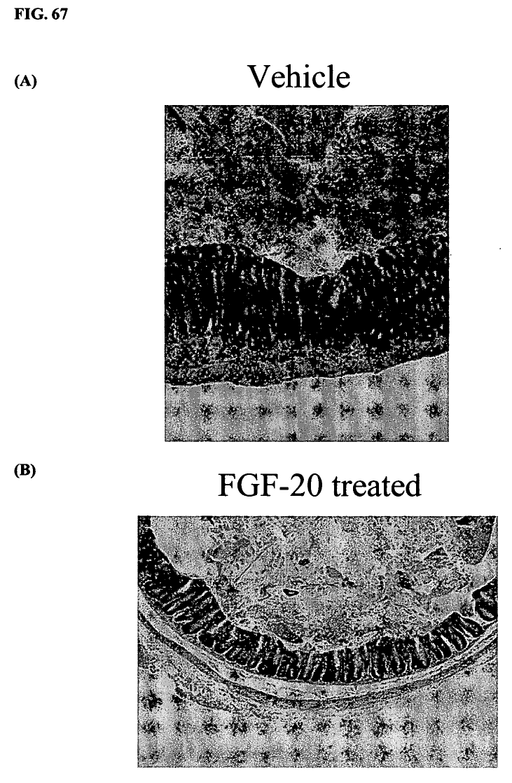

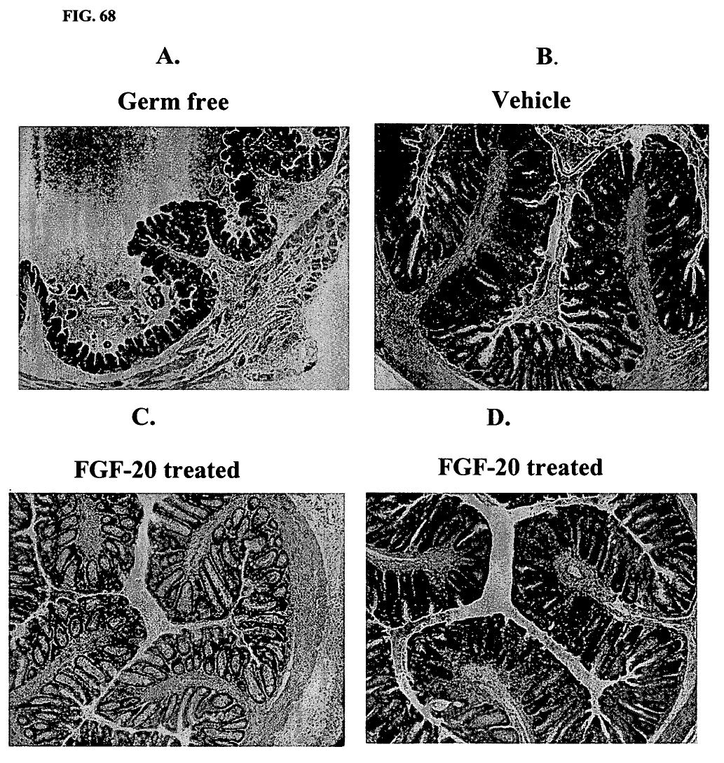

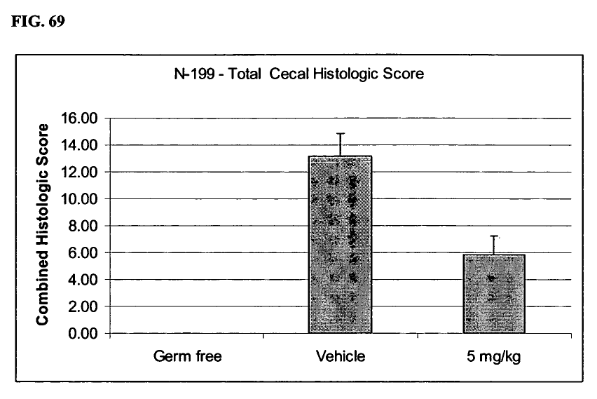

FIG. 66 shows the Weight change in the treatment study, where FGF-20 (5 mg/kg) was administered to IL-10 KO mice. Histology of the cecum and rectum are respectively shown in FIGS. 67 and 68, that demonstrates protective effect of FGF-20. Cecal histologic score shows that FGF-20 decreased as compared to the vehicle control (13.1±1.8 vs. 5.9±1.4; p<0.006, FIG. 69).

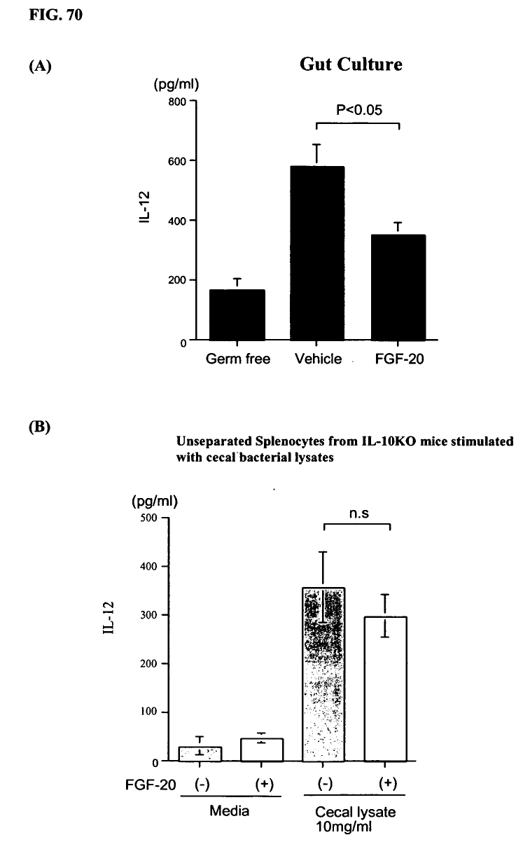

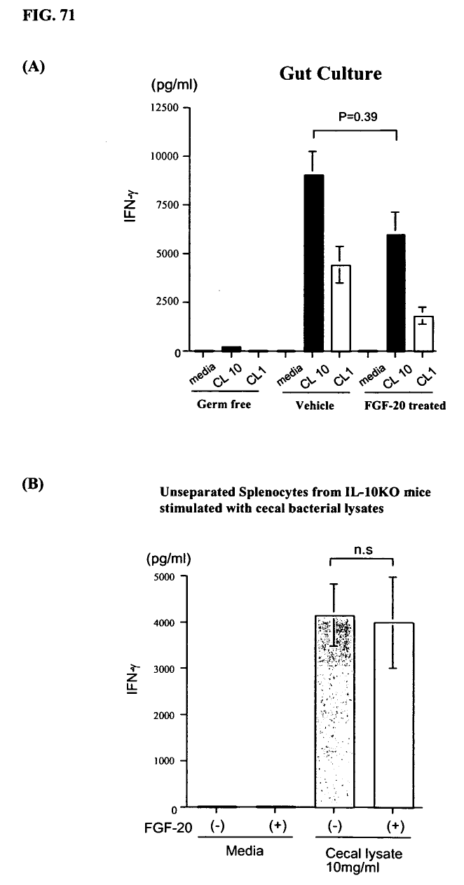

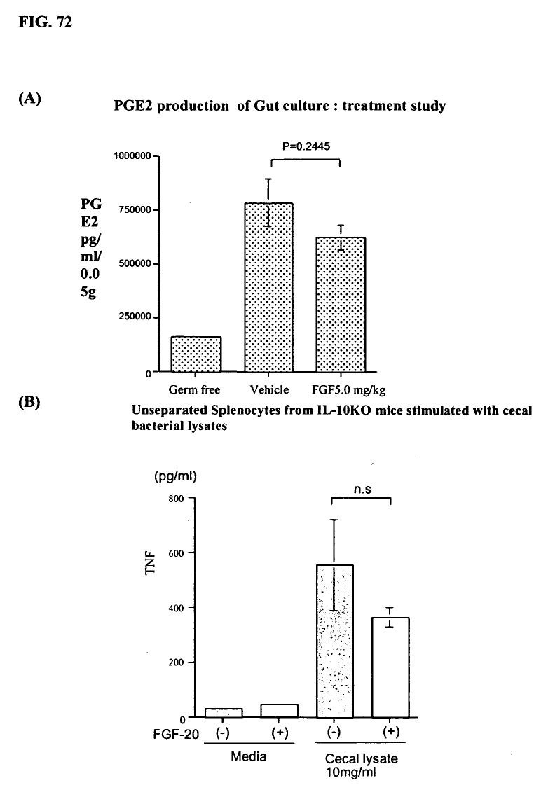

Cytokine production in treatment group as assayed by ELISA demonstrated that FGF-20 administration did not significantly alter the cytokine production in Gut culture and unsepartated splenocytes of IL-10 KO mice. IL-12 (FIG. 86), IFN-? (FIG. 70), TNF-a (FIG. 71) and PGE2 (FIG. 71) were the cytokines that were assayed. FIG. 72 shows FACS analysis of MLN number, CD4+ and CD8+ and CD69+ cells, all of which were decreased in FGF-20 treated group as compared to the vehicle treatment.

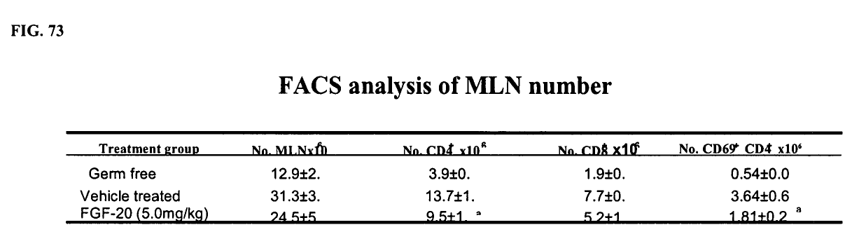

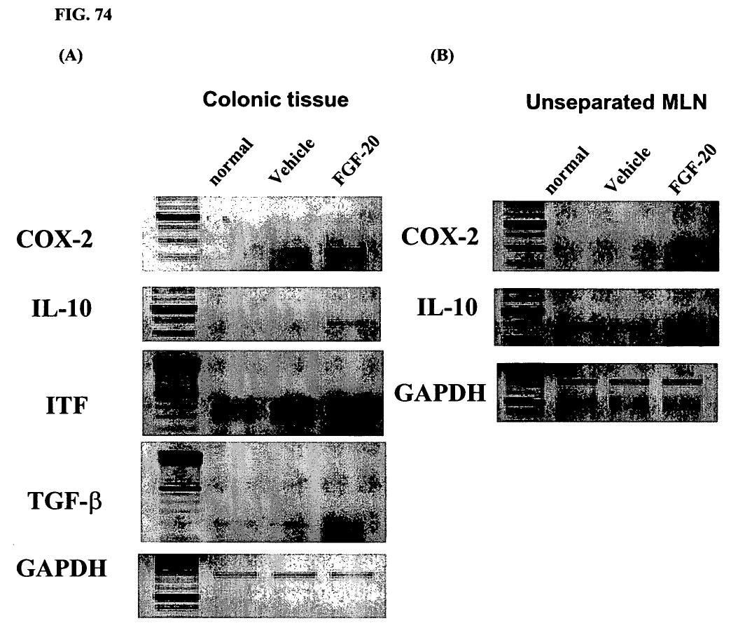

Results in normal mice: Expression of COX-2, IL-10, ITF, TGF-□ were analyzed in normal wild type (WT) C57BL6 mice following 7 days of injection FGF-20 (5 mg/kg). RT-PCR was performed (as described in Example 7) in colonic tissue and unseparated MLN to study the expression of the above list of genes. FIG. 73 shows that COX-2, IL-10, ITF and TGF-β are upregulated in the colonic tissue of WT mice, upon administering FGF-20. In unseparated MLN, IL-10 expression is found to be upregulated as compared to vehicle (FIG. 73).

### 6.17 Example 17

### Effect of CG53135-05 in a Chronic 2 Week Murine Model of DSS-Induced Ulcerative Colitis (N-404)

Female Swiss Webster mice exposed to dextran sulfate sodium (DSS) for 7 days develop inflammation and gland loss with erosion in the colon. Gross and histopathologic changes resulting from this treatment resemble those occurring in human ulcerative colitis (UC), a subset of inflammatory bowel disease (IBD) (see Animal Models of Intestinal Erosion in “Inflammatory Bowel Disease” ed. MacDermott R P and Stenson W F. Elsevier, New York (1992); Okayasu et al., Gastroenterology 98: 694-702 (1990); and Cooper et al., Lab Invest. 69: 238-249 (1993)). Compounds that are effective in the treatment of human IBD have activity in this model and it is being used to investigate potential new therapies (see Axelsson et al., Aliment Pharmacol Ther. 12: 925-934 (1998); Egger et al., Digestive Dis Sci. 44: 436-444 (1999); Miceli et al., J Pharmacol Exp Ther. 290: 464-471 (1999); and Jeffers et al., Gastroenterology 123 (4): 1151-62 (2002)). However, the 7-day version of the model, in which exposure to DSS is continuous, is only useful for evaluating the effects of agents on the acute phase of mucosal inflammation and damage. A more chronic model of ulcerative colitis, with less potential for lethality, was developed to test CG53135-05 for its capacity to enhance mucosal repair. The objective of this study was to assess the potential therapeutic activity of CG53135-05 in the chronic 2 week murine model of ulcerative colitis induced by administration of 3% (DSS) for 5 days and 1% DSS for 9 days.

Materials and Methods:

Colitis was induced in female Swiss Webster mice by exposure to 3% DSS in the drinking water (ad libitum) on study Days 1-5 and maintained by exposure to 1% DSS in the drinking water (ad libitum) on study Days 6-15. Mice were randomly assigned to 4 groups of 15 animals (Table X). Mice were exposed to 3% DSS in drinking water on test Days 1 through 5 and 1% DSS on test Days 6-15, with concurrent intraperitoneal (IP) treatments of vehicle or CG53135-05 at 0.33, 1.67 and 3.33 mg/kg (UV) Days 6-14 after disease was established. Mice were weighed daily. On Day 15 animals were terminated by cervical dislocation, the colon length was measured, and colon content was scored. Tissues were collected into 10% neutral buffered formalin and processed routinely for histopathology.

TABLE 15
Study Design

| | | 3% DSS | 1% DSS | | |
|---|---|---|---|---|---|
| | | Treatment | Treatment | CG53135-05 | CG53135-05 |
| Group | Number of | (water po), | (water po), | Treatment | Treatment Schedule |
| Number | Animals* | Days 1-5 | Days 6-15 | (mg/kg, UV) | (IP) |
| 1 | 15 females | Yes | Yes | 0 | Vehicle only, Days 6-14 |
| 2 | 15 females | Yes | Yes | 0.33 | Once daily, Days 6-14 |
| 3 | 15 females | Yes | Yes | 1.67 | Once daily, Days 6-14 |
| 4 | 15 females | Yes | Yes | 3.33 | Once daily, Days 6-14 |

*Acclimation of animals at least 1 week prior to randomization and start of treatment

Clinical Parameters and Gross Pathology: Only animals that survived the duration of the study were included in the analysis of body weight change, terminal colon lengths, and colon content scores. At necropsy, the colon length was measured and assessed for evidence of stool consistency changes. Colon content was scored at necropsy according to the following criteria: 0=Normal (firm, well formed stool); 1=Semi-solid stool; 2=Semi-solid to fluid stool; and 3=Semi-solid with definite evidence of blood, bloody fluid or no content.

Histopathology: At necropsy, the colon was harvested and divided into 2 approximately equal segments, proximal and distal, to assess regional changes induced in this model. Distal ends were marked to maintain orientation. These colon segments were collected, preserved in 10% neutral buffered formalin, and routinely processed for histopathologic evaluation. During processing for histology, the proximal and distal colon segments were each trimmed to obtain 4 equally spaced segments, and hematoxylin and eosin (H&E) stained slides were prepared for each. Both proximal and distal tissues were examined as these tissues are affected to different degrees of severity in this model (more severe and less variable symptoms predominate in the distal colon).

For each colon section, submucosal edema was quantified by measuring the distance (mm) from the muscularis mucosa to the internal border of the outer muscle layer. The extent of inflammation (foamy macrophages, lymphocytes and polymorphonuclear cell infiltrate) was assigned severity scores according to the following ranking: 0=Normal; 1=Minimal; 2=Mild; 3=Moderate; 4=Marked; and 5=Severe.

The parameters reflecting epithelial cell loss/damage were scored individually using a percent area involved scoring method. Parameters that were scored using the percent involvement scale included colon glandular loss and colon erosion.
0=None
1=1-10% of the mucosa affected
2=11-25% of the mucosa affected
3=26-50% of the mucosa affected
4=51-75% of the mucosa affected
5=76-100% of the mucosa affected

Mucosal epithelial hyperplasia (basophilia, mitotic figures, multilayered on basement membranes, absence of goblet cells) was scored 0-5 based on the following criteria
0=Normal
1=Minimal-small foci generally adjacent to the inflammatory changes
2=Mild-11-25% of mucosa affected
3=Moderate-26-50% of mucosa affected
4=Marked-51-100% of mucosa affected
5=Severe-51-100% of mucosa affected plus papillary proliferation into lumen

Mucosal thickness (an indicator of proliferative changes or edematous inflammatory mucosal expansion) was measured by placing an ocular micrometer at the base of the glands and the overlying surface epithelium in a non-tangential area of section representative of the thickest areas of mucosa.

The scores for proximal tissues and distal colon were averaged to determine a score for the entire colon for each parameter evaluated. The 3 scored parameters (i.e., inflammation, glandular loss, and surface epithelial erosion) were combined to arrive at a sum of overall histopathology scores for proximal or distal sections of the colon, and then proximal and distal overall scores were averaged to arrive at an overall histopathology score for the entire colon. These summations indicate the overall damage in the distal, proximal, or entire colon and would have a maximum severity score of 15.

Additional tissues (cheek, tongue, esophagus, mid colon and rectum) were collected and divided into two equal sections. One section was snap frozen in liquid nitrogen, the other was placed in 10% neutral buffered formalin for paraffin embedding and immunohistochemical staining with Ki67 antibody. The formalin preserved tissues were trimmed into approximately 0.5 cm sections (1 section/tissue), processed through graded alcohols and a clearing agent, infiltrated, embedded in paraffin and sectioned. Slides were then re-hydrated and stained with mouse Ki67 antibody (Dako) at 1:70 dilution followed by streptavidin/HRP detection system with a DAB peroxidase indicator and counterstained with Gill's Hematoxylin. The slides were exposed to diaminobenzadine (DAB) for four minutes to provide optimal specific staining with minimal background nonspecific staining.

Statistics: Differences in body weight changes between treatment groups were analyzed with repeated measures ANOVA with a Greenhouse-Geisser correction for sphericity, followed by pair-wise repeated measures analysis to identify the source of any variation. These pair-wise measures were performed between each day and the normal control. Quantitative measurements of pathology (colon length, edema) were analyzed with a one-way analysis of variance (ANOVA) followed by a linear contrast. Qualitative measures of pathology (histology scores) were analyzed as follows: Pathological evaluations were done at several points along the proximal and distal colon. These replicates were converted to a mean pathology score and a maximal pathology score for each individual to correct for problems of pseudo-replication. The mean and maximal scores were analyzed with a Kruskal-Wallis test followed by a Dunn's multiple comparison test between each day and normal control animals. In all other cases, multiple comparison test results were adjusted for the number of comparisons being performed using a Bonferonni correction. In the case of pathology scores, the comparisons were effectively doubled, by analyzing the mean and the maximum values.

Results:

Fifty-nine of the sixty mice survived the duration of the study and were included in the analysis of body weight loss, terminal colon lengths, and colon content. Decreased body weight gain was seen in all groups with the most severe effects occurring on Day 9 and recovery of gain beginning on Day 10. A dose-dependent trend in inhibition of DSS-induced body weight loss was observed, corresponding to 19, 35 and 67% reduction in weight loss for groups receiving IP injections of 0.33, 1.67 or 3.33 mg/kg (UV) CG53135-05, respectively. Gross pathology evaluation indicated that colon length decreases and colon content score increases were greatest in the vehicle-treated disease control group. Dose-responsive increases in colon length were seen in all groups treated with CG53135-05 IP, with the greatest effect (29% inhibition, statistically significant) occurring in mice administered 3.33 mg/kg (UV) CG53135-05. Colon scores were also significantly improved (21-29%) in mice treated with 1.67 or 3.33 mg/kg (UV) CG53135-05.

Disease vehicle-treated control mice all had lesions of colitis with minimal to severe inflammation and gland loss. Erosion and gland hyperplasia were evident in 14 of 15 mice with these changes being greatest in the distal colon. Edema was sporadically seen, primarily in the distal colon. Distal colon mucosal thickness was increased approximately 130% in diseased controls as compared to normal controls, as a result of inflammation and hyperplasia. In contrast, dose-responsive inhibition of proximal, distal, and total colon inflammation and gland loss as well as summed total colon histopathologic scores was seen. Furthermore, mice that received 3.33 mg/kg (UV) CG53135-05 showed significant improvements in on (35%) and total (34%) inflammation, distal (38%) and total (37%) gland loss as well as distal (38%) and total (37%) summed histopathologic scores. Protective effects (46-62% inhibition, non-significant) of CG53135-05 were also seen on proximal, distal and total colon erosion in the mid and high dose groups. Beneficial effects (dose responsive, non-significant) on inhibition of mucosal thickness changes and hyperplasia scores occurred with 39% inhibition of the total mucosal thickness in mice treated with 3.33 mg/kg (UV) and 31% improvement in the hyperplasia score.

Conclusions:

In summary, treatment with CG53135-05 IP, once daily at doses of 1.67 or 3.33 mg/kg (UV) resulted in mild to moderate, dose-responsive, inhibitory effects on gross and histopathologic parameters in the 15-day chronic model of DSS-induced colitis, when treatment was initiated after disease was established. Colon length, mean total gland loss, distal colon mean histology summed score, max distal and total inflammation, and max distal and total gland loss showed a significant linear dose response and were significantly different between animals in the vehicle-treated control group and the group treated with 3.33 mg/kg (UV) CG53135-05.

In another experiment, administration of 3.33 mg/kg (UV) of CG53135-05 IP, once every other day on Days 6, 8, 10, 12, and 14 (q2d) significantly reduced the severity of chronic DSS-induced ulcerative colitis in female Swiss Webster mice (N-405 study). These results confirm the findings presented above.

### 6.18 Example 18

Manufacture of CG53135-05 and Pharmaceutical Formulations

Aiming for a construct that would be suitable for clinical development, untagged molecules were generated in a phage-free bacterial host. The codon-optimized, full-length, untagged molecule (CG53135-05) has the most favorable pharmacology profile and was used to prepare product for the safety studies and clinical trial.

### 6.18.1 Production Process and Pharmaceutical Formulations (Process 1)

CG53135-05 was expressed in Escherichia coli BLR (DE3) using a codon-optimized construct, purified to homogeneity, and characterized by standard protein chemistry techniques. The isolated CG53135-05 protein migrated as a single band (23 kilodalton) using standard SDS-PAGE techniques and stained with Coommassie blue. The CG53135-05 protein was electrophoretically transferred to a polyvinylidenefluoride membrane and the stained 23 kD band was excised from the membrane and analyzed by an automated Edman sequencer (Procise, Applied Biosystems, Foster City, Calif.); the N-terminal amino acid sequence of the first 10 amino acids was confirmed as identical to the predicted protein sequence.

Fermentation and Primary Recovery Recombinant

CG53135-05 was expressed using Escherichia coli BLR (DE3) cells (Novagen). These cells were transformed with full length, codon optimized CG53135-05 using pET24a vector (Novagen). A Manufacturing Master Cell Bank (MMCB) of these cells was produced and qualified. The fermentation and primary recovery processes were performed at the 100 L (i.e., working volume) scale reproducibly.

Seed preparation was started by thawing and pooling of 1-6 vials of the MMCB and inoculating 4-7 shake flasks each containing 750 mL of seed medium. At this point, 3-6 L of inoculum was transferred to a production fermentor containing 60-80 L of start-up medium. The production fermentor was operated at a temperature of 37° C. and pH of 7.1. Dissolved oxygen was controlled at 30% of saturation concentration or above by manipulating agitation speed, air sparging rate and enrichment of air with pure oxygen. Addition of feed medium was initiated at a cell density of 30-40 AU (600 nm) and maintained until end of fermentation. The cells were induced at a cell density of 40-50 AU (600 nm) using 1 mM isopropyl-beta-D-thiogalactoside (IPTG) and CG53135-05 protein was produced for 4 hours post-induction. The fermentation was completed in 10-14 hours and about 100-110 L of cell broth was concentrated using a continuous centrifuge. The resulting cell paste was stored frozen at −70° C.

The frozen cell paste was suspended in lysis buffer (containing 3M urea, final concentration) and disrupted by high-pressure homogenization. The cell lysate was clarified using continuous flow centrifugation. The resulting clarified lysate was directly loaded onto a SP-sepharose Fast Flow column equilibrated with SP equilibration buffer (3 M urea, 100 mM sodium phosphate, 20 mM sodium chloride, 5 mM EDTA, pH 7.4). CG53135-05 protein was eluted from the column using SP elution buffer (100 mM sodium citrate, 1 M arginine, 5 mM EDTA, pH 6.0). The collected material was then diluted with an equal volume of SP elution buffer. After thorough mixing, the SP Sepharose FF pool was filtered through a 0.2 μm PES filter and frozen at −80° C.

Purification of the Drug Substance

The SP-sepharose Fast Flow pool was precipitated with ammonium sulfate. After overnight incubation at 4° C., the precipitate was collected by bottle centrifugation and subsequently solubilized in Phenyl loading buffer (100 mM sodium citrate, 500 mM L-arginine, 750 mM NaCl, 5 mM EDTA, pH 6.0). The resulting solution was filtered through a 0.45 uM PES filter and loaded onto a Phenyl-sepharose HP column. After washing the column, the protein was eluted with a linear gradient with Phenyl elution buffer (100 mM sodium citrate, 500 mM L-arginine, 5 mM EDTA, pH 6.0). The Phenyl-sepharose HP pool was filtered through a 0.2 μm PES filter and frozen at −80° C. in 1.8 L aliquots.

Formulation and Fill/Finish

Four batches of purified drug substance were thawed for 24-48 hours at 2-8° C. and pooled into the collection tank of tangential flow ultrafiltration (TFF) equipment. The pooled drug substance was concentrated ˜5-fold via TFF, followed by about 5-fold diafiltration with the formulation buffer (40 mM sodium acetate, 0.2 M L-arginine, 3% glycerol). This buffer-exchanged drug substance was concentrated further to a target concentration of >10 mg/mL. Upon transfer to a collection tank, the concentration was adjusted to ˜10 mg/mL with formulation buffer. The formulated drug product was sterile-filtered into a sterile tank and aseptically filled (at 10.5 mL per 20 mL vial) and sealed. The filled and sealed vials were inspected for fill accuracy and visual defects. A specified number of vials were drawn and labeled for release assays, stability studies, safety studies, and retained samples. The remaining vials were labeled for the clinical study, and finished drug product was stored at −80±15° C.

The finished drug product is a sterile, clear, colorless solution in single-use sterile vials for injection. CG53135-05 E. coli purified product was formulated at a final concentration of 8.2 mg/mL (Table 16).

TABLE 16
Composition of Drug Product

| | | | |
|---|---|---|---|
| | | Final | Amount |
| Component | Grade | concentration | per Liter |

| | | | | | |
|---|---|---|---|---|---|
| CG53135-05 E. coli | NA | 8.2 | mg/ml | 8.2 | g |
| purified product | | | | | |
| Formulation Buffer | | | | | |
| Sodium acetate | USP | 40 | mM | 5.44 | g |
| (trihydrate) | | | | | |
| L-arginine HCl | USP | 200 | mM | 42.132 | g |
| Glycerol | USP | 3% | v/v | 30 | mL |

| Acetic acid | USP | NA | QS to | |
|---|---|---|---|---|
| | | | pH | 5.3 |
| Water for injection | USP | NA | QS to | |
| | | | 1 | L |

The pharmacokinetics of optimally-formulated CG53135-05 E. coli purified product was assessed in rats following intravenous, subcutaneous, and intraperitoneal administration to compare exposure at active doses in animal models and predict exposure in humans. Intravenous administration of CG53135-05 E. coli purified product resulted in high plasma levels (maximum plasma level=19,68047,252 ng/mL), which rapidly declined within the first 2 hours to 30-70 ng/mL; decreased exposure was observed following the third daily dose (maximum plasma level=5373-7453 ng/mL). Subcutaneous administration of CG53135-05 E. coli purified product resulted in slow absorption (maximum plasma level at 10 hours) and plasma levels of 40-80 ng/mL up to 48 hours after dosing; some accumulation in plasma was seen following the third daily dose. Intraperitoneal administration of CG53135-05 E. coli purified product resulted in slow absorption (maximum plasma level at 2-4 hours) and plasma levels of 40-70 ng/mL up to 10 hours after dosing; decreased exposure was seen following third daily dose. No significant gender differences were observed by any route of administration.

Safety of intravenous administration of CG53135-05 E. coli purified product (0.05, 5 or 50 mg/kg/day for 14 consecutive days) was assessed in a pivotal toxicology study in rats. There were no treatment-related findings in rats administered 0.05 mg/mL CG53135-05 E. coli purified product for 14 days. In rats administered 5 mg/kg CG53135 for 14 days, food consumption was reduced and body weight was decreased; while there were no treatment-related changes in organ weights, urinalysis, ophthalmology, or histopathology parameters in this dose group, there were treatment-related changes in hematology and clinical chemistry parameters in this treatment group. In rats administered 50 mg/kg CG53135-05 E. coli purified product for 12 days (estimated maximum plasma level of 20-30 fold higher than active dose), food consumption was reduced and body weight was markedly decreased; while there were no treatment-related changes in ophthalmology, there were significant treatment-related changes in organ weights, urinalysis, hematology, clinical chemistry, and histopathology in this treatment group.

Safety of intravenous administration of CG53135-05 E. coli purified product (0 or 10 mg/kg/day for 7 consecutive days) was further assessed in a safety pharmacology study in rhesus monkeys. There were no treatment-related clinical observations in animals administered 1 mg/kg CG53135-05 E. coli purified product for 7 days. In animals administered 10 mg/kg CG53135-05 E. coli purified product for 7 days, minor effects on body weight were noted and associated with qualitative observations of lower food consumption. There were no apparent treatment-related effects on hematology, clinical chemistry, ophthalmology, or electrophysiology in either dose group.

Stability of CG53135-05 Drug Substances

Stability studies on the CG53135-05 E. coli purified product produced during cGMP manufacturing were performed. The analytical methods used as stability indicating assays for purified drug substance are listed in Table 17.

TABLE 17
Stability Assays for Drug Substance

| Assay | Stability Criteria |
|---|---|
| SDS-PAGE (Neuhoff stain) | >98% pure by densitometry (reduced |
| | and nonreduced) |
| RP-HPLC | Peak at 5.5 ± 1.0 min relative |
| | retention time |
| SEC-HPLC | >90% mono-disperse peak |
| Total protein by | >0.2 mg/mL |
| Bradford method | |
| Bioassay (BrdU) | PI₂₀₀ > 0.5 ng/mL and < 20 ng/mL |
| pH | 5.8 ± 0.4 |
| Visual appearance | Clear and colorless |

PI₂₀₀ = concentration of CG53135-05 that results in incorporation of BrdU at 2 times the background

The SDS-PAGE, RP-HPLC, and Bradford assays are indicative of protein degradation or gross aggregation. The SEC-HPLC assay detects aggregation of the protein or changes in oligomerization, and the bioassay detects loss of biological activity of the protein. The stability studies for the purified drug substance were conducted at −80 to 15° C. with samples tested at intervals of 3, 6, 9, 12, and 24 months.

In one experiment, stability studies of finished drug product were conducted by Cambrex at −80±15° C. and −20±5° C. with samples tested at intervals of 1, 3, 6, 9, 12, and 24 months. Stability data collected after 1 month indicate that finished drug product is stable for at least 1 month when stored at −80±15° C. or at −20±5° C. (Table 18).

TABLE 18
Stability Data for Drug Product after 1-month interval

| Assay | Stability Criteria | Initial | −80 ± 15° C. | −20 ± 5° C. |
|---|---|---|---|---|
| RP-HPLC | Major peak | Major peak | Major peak | Major peak |
| | retention time ± 0.2 | retention time ± | retention time ± | retention time ± |
| | min relative to | 0.2 min relative | 0.2 min relative | 0.2 min relative |
| | Reference Standard | to Reference | to Reference | to Reference |
| | | Standard | Standard | Standard |
| SDS-PAGE | Major band | Pass | Pass | Pass |
| | migrates at about | | | |
| | 23 kDa; | | | |
| | nonreduced minor | | | |
| | band below major | | | |
| | band | | | |
| SEC-HPLC | >90% mono-disperse | 100% | 100% | 100% |
| | peak | | | |
| Bradford | 10 ± 0.2 mg/mL | 8.2 | 8.6 | 8.3 |
| Bioassay | PI₂₀₀ > 0.5 ng/mL | 4.14 ng/mL | 2.98 ng/mL | 1/45 ng/mL |
| | and < 20 ng/mL | | | |
| Sterility | Pass (i.e., no | Pass | NT | NT |
| | growth) | | | |
| pH | 5.3 ± 0.3 | 5.4 | 5.5 | 5.4 |
| Visual | Clear and colorless | Pass | Pass | Pass |
| appearance | solution | | | |

Lot # 02502001 was stored at −80±15° C. or at −20±5° C. at Cambrex and tested after 1 month; PI200=concentration of CG53135-05 that results in incorporation of BrdU at 2 times the background; Pass=results met stability criterion; NT=not tested.

In another experiment, samples of finished drug product were stored at −80±15° C. or stressed at 5±3° C., 25±2° C., or 37±2° C. and tested at various intervals for 1 month. Stability data indicate that finished drug product showed no significant instability after 1 month of storage at −80±15° C. or 5±3° C. When stressed at 25±2° C., finished drug product was stable for at least 48 hours; degradation was apparent after 1 week at this temperature. When stressed at 37±2° C., degradation of finished drug product was apparent within 4 hours.

### 6.18.2 Improved Pharmaceutical Formulations and Production Process of CG53135-05 (Process 2)

A new formulation was developed to meet the three requirements for a commercial product: (1) the minimal storage temperature should be 2-8° C. for ease of distribution; (2) product should be stable at the storage temperature for at least 18 months for a commercial distribution system; and (3) product should be manufactured by commercial scale equipment, and processes should be transferable to various commercial contract manufacturers.

The new formulation consists 10 mg/mL of the protein product produced by the process described in Section 6.2 (“Process 2 protein”) in 0.5 M arginine as sulfate salt, 0.05 M sodium phosphate monobasic, and 0.01% (w/v) polysorbate 80. The lyophilized product is projected to be stable for at least 18 months at 2-8° C. based on accelerated stability data. In contrast to the new formulation, the previous formulation as described in U.S. application Ser. No. 10/435,087 is not possible to be lyophilized for the following reasons: firstly, the acidic component of the acetate buffer is acetic acid, which sublimes during lyophilization. This loss of acetic acid to lyophilization increases the pH to >7.5, which is far from the target pH of 5.3. Secondly, the glycerol has a collapse temperature of <−45° C., which renders this formulation not be able to be lyophilized commercially. Most of the commercial lyophilizers have a shelf temperature ranged from −45° C. to −50° C. with temperature variation of ±3° C.

Four unexpected properties of CG53135 were discovered and used to develop the new formulation: (1) high concentration of arginine, >0.4 M, increases the solubility to >30 mg/mL; (2) the use of sulfate salt of arginine increases the solubility by at least 2-6 folds; (3) the optimal concentration of sodium phosphate as a buffering salt is 50 mM, with a solubility of at least 1-2 fold increase in comparison with concentrations at 25, 75, and 100 mM; and (4) adding a surfactant during the diafiltration/ultrafiltration step minimizes the formation of aggregates. In development the lyophilized formulation, each component of the new formulation was evaluated for solubility individually. CG53135-05 was precipitated using the precipitate buffer (50 mM NaPi, 5 mM EDTA, 1 M L-Arginine HCl, 2.5 M (NH4)₂SO4). The precipitate was washed with 25 mM sodium phosphate buffer at pH 6.5 to remove the residual arginine and ammonium sulfate. The washed precipitate was then re-dissolved in the following respective buffers listed in the tables. The following are examples of data.

TABLE 19
High concentration of arginine, >0.4 M, increases the solubility to >30 mg/mL

| Concenctration of | Solubility of Process 2 protein in mg/mL | | | | |
|---|---|---|---|---|---|
| Arginine (M) | Batch #1 | Batch #2 | Batch #3 | Batch #4 | Batch #5 |
| 0.05 | 0.7 | 0.6 | 0.5 | ND | ND |
| 0.10 | 1.4 | 0.6 | 1.2 | ND | ND |
| 0.15 | 2.2 | 1.6 | 2.2 | ND | ND |
| 0.20 | 3.0 | 4.7 | 4.3 | ND | ND |
| 0.30 | ND | ND | ND | 5.8 | ND |
| 0.35 | ND | ND | ND | 10.1 | ND |
| 0.40 | ND | ND | ND | 9.8 | ND |
| 0.45 | ND | ND | ND | 32.3 | ND |
| 0.50 | ND | ND | ND | 23.8* | 37 |

*The solubility was lower as there was not sufficient protein in the experiment to be dissolved.

TABLE 20
The use of sulfate salt of arginine increases the solubility by at least 2-6 folds.

| Concentration of | | | | | | |
|---|---|---|---|---|---|---|
| sodium phosphate | | Solubility of Process 2 protein in mg/mL | | | | |
| monobasic* | | Batch #A | Batch #B | Batch #C | Batch #D | Batch #E |
| 100 | mM | 3.78 | 2.8 | 2.4 | 2.9 | 2.47 |
| 75 | mM | 4.06 | 2.5 | 2.6 | 3.0 | 2.38 |
| 50 | mM | 5.47 | 4.7 | 3.3 | 4.3 | 4.81 |
| 25 | mM | 4.01 | 2.4 | 2.6 | 2.4 | 3.59 |

All formulation contains 0.2 M arginine.

An optimal concentration of the sodium phosphate as a buffering salt was observed (Table 12). The optimal concentration of sodium phosphate is 50 mM with a solubility of at least 1-2 fold increase in comparison with concentrations at 25, 75, and 100 Mm.

TABLE 21
The optimal concentration of sodium phosphate
as a buffering salt is 50 mM

| | Solubility Increament of Process 2 protein | |
|---|---|---|
| | in using Arginine Sulfate vs Arginine | |
| | Phosphate in mg/mL | |
| Formulation | Batch #K | Batch # J |
| 50 mM sodium phosphate | 4.4 | 2.3 |
| monobasic and 0.15 M | | |
| Arginine at pH 7 | | |
| 50 mM sodium phosphate | 6.5 | 5.2 |
| monobasic and 0.15 M | | |
| Arginine at pH 7 | | |

Table 22 shows a need to add a surfactant during the diafiltration/ultrafiltration step to minimize the formation of aggregates. The experiment was conducted by performing the ultrafiltration/diafiltration at 2.5 mg/mL CG53135-05 in 0.2M arginine and 0.05 M sodium phosphate buffer at pH 7.0. After exchanging with 7 volumes of the final buffer (0.5M arginine and 0.05 M sodium phosphate buffer at pH 7.0), the diafiltrate is concentrated to ˜20 mg/mL. The diafiltrate is then diluted with the final buffer to ˜12.5 mg/mL and lyophilized. Polysorbate 80 is added either before or after the diafiltration to a final concentration of 0.01%.

TABLE 22
Adding a surfactant during the diafiltration/ultrafiltration
step minimizes the formation of aggregates.

| Polysorbate added during | Process 2 protein | Turbidity |
|---|---|---|
| ultrafiltration/diafiltration | Concentration (mg/mL) | (NTU) |
| Yes | 12.5 | 20.9 |
| No | 13.0 | 4.6 |

All formulation contains 0.5 M arginine, 0.05 M sodium phosphate monobasic, and 0.01% polysorbate 80.

The new formulation has the following advantages: (1) a lyophilized product with a storage temperature of 2-8° C.; (2) a lyophilized product with a projected shelf-life of at least 18 months when stored at 2-8° C. achieve the solubility of >30 mg/mL; and (3) The lyophilized product has a collapse temperature of −30° C. which can be easily lyophilized by the commercial equipment. The interactions between arginine, sulfate, phosphate, and surfactant and CG53135 were unexpected.

The improved process steps for the manufacturing of drug substance and drug product are described in Table 23, and each step is explained below.

TABLE 23
Manufacturing Process
Ampoule from WCB
↓
Seed Flask and Seed Fermenter 25 L - Innoc
↓
Fermentation at 1500 L scale
↓
Homogenization + 0.033% PEI or a charged
heterogenous polymer
↓
Purification by SP Streamline
↓
Purification by PPG 650 M
↓
Cuno Filtration
↓
Purification by Phenyl Sepharose HP
↓
Concentration/Diafiltration addition of 0.01%
polysorbate 80 or Polysorbate 20
↓
Bottling - Drug Substance
↓
QC Testing and Release
↓
Sterile Vial Fill & Lyophilization
↓
Drug Product
↓
QC Testing and Release

Cell Bank: a Manufacturing Master Cell Bank (MMCB) in animal component free complex medium was used in an earlier Process. A second Manufacturing Master Cell Bank (MMCB) in animal component free chemically defined medium was derived from the first MMCB and a Manufacturing Working Cell Bank (MWCB) was made from the second MMCB. This MWCB was used in the manufacturing process as described in Table 23.

Innoculum Preparation: the initial cell expansion occurs in shake flasks. Seed preparation is done by thawing and pooling 2-3 vials of the MWCB in chemically defined medium and inoculating 3-4 shake flasks each containing 500 mL of chemically defined seed medium.

Seed and Final Fermentation: the shake flasks with cells in exponential growth phase (2.5-4.5 OD600 units) are used to inoculate a single 25 L (i.e., working volume) seed fermenter containing the seed medium. The cells upon reaching exponential growth phase (3.0-5.0 OD600 units) in the 25 L seed fermenter are transferred to a 1500 L production fermenter with 780-820 L of chemically defined batch medium. During fermentation, the temperature is controlled at 37±2° C., pH at 7.1±0.1, agitation at 150-250 rpm and sparging with 0.5-1.5 (vvm) of air or oxygen-enriched air to control dissolved oxygen at 25% or above. Antifoam agent (Fermax adjuvant 27) is used as needed to control foaming in the fermenter. When the OD (at 600 nm) of culture reaches 25-35 units, additional chemically defined medium is fed at 0.7 g/kg broth/min initially and then with feed rate adjustment as needed. The induction for expression of CG53135-05 protein is started when OD at 600 nm reaches 135-165 units. After 4 hours post-induction the fermentation is completed. The final fermentation broth volume is approximately 1500 L. The culture is then chilled to 10-15° C.

Homogenization: the chilled culture is diluted with cell lysis buffer at the ratio of one part of fermentation broth to two parts of cell lysis buffer (50 mM sodium phosphate, 60 mM EDTA, 7.5 mM DTT, 4.5 M urea, pH 7.2. Polyethyleneimine (PEI), a flocculating agent is added to the diluted fermentation broth to a final PEI concentration at 0.033% (W/V). The cells are lysed at 10-15° C. with 3 passages through a high-pressure homogenizer at 750-850 bar.

Capture and Recovery: the chilled cell lysate is directly loaded in the upflow direction onto a pre-equilibrated Streamline SP expanded bed cation exchange column. During the loading, the bed expansion factor is maintained between 2.5-3.0 times the packed bed column volume. After loading, the column is flushed with additional Streamline SP equilibration buffer (100 mM sodium phosphate, 40 mM EDTA, 10 mM sodium sulfate, 3 M urea, pH 7.0) in the upflow direction. The column is then washed further with SP Streamline wash buffer (100 mM sodium phosphate, 5 mM EDTA, 25 mM sodium sulfate, 2.22 M dextrose, pH 7.0) in the downflow direction. The protein is eluted from the column with Streamline SP elution buffer (100 mM sodium phosphate, 5 mM EDTA, 200 mM sodium sulfate, 1 M L-arginine, pH 7.0) in the downflow direction.

PPG 650M Chromatography: the SP Streamline eluate is loaded on to a pre-equilibrated PPG 650 M, hydrophobic interaction chromatography column. The column is equilibrated and washed with 100 mM sodium phosphate, 200 mM sodium sulfate, 5 mM EDTA, 1 M Arginine pH 7.0. The column is further washed with 100 mM sodium phosphate, 5 mM EDTA, 0.9 M Arginine, pH 7.0. The product is eluted with 100 mM sodium phosphate, 5 mM EDTA, 0.2 M Arginine, pH 7.0.

CUNO Filtration: the PPG eluate is passed through an endotoxin binding CUNO 30ZA depth filter. The filter is flushed first with water for injection (WFI) and then with 100 mM sodium phosphate, 5 mM EDTA, 0.2 M Arginine, pH 7.0 (PPG eluate buffer). After flushing, the PPG eluate is passed through the filter. Air pressure is used to push the final liquid through the filter and its housing.

Phenyl Sepharose Chromatography: the CUNO filtrate is then loaded on to a pre-equilibrated Phenyl Sepharose hydrophobic interaction chromatography column. The column is equilibrated and washed with 100 mM sodium phosphate, 50 mM ammonium sulfate, 800 mM sodium chloride, 0.5 M Arginine, pH 7.0. The product is eluted with 50 mM sodium phosphate, 0.5 M Arginine, pH 7.0.

Concentration and Diafiltration: a 1% Polysorbate 80 is added to the Phenyl Sepharose eluate so that the final concentration in the drug substance is 0.01% (w/v). The eluate is then concentrated in an ultrafiltration system to about 2-3 g/L. The retentate is then diafiltered with 7 diafiltration volumes of 50 mM sodium phosphate, 0.5 M Arginine, pH 7.0 (Phenyl Sepharose elution buffer). After diafiltration the retentate is concentrated between 12-15 g/L. The retentate is filtered through a 0.22 μm filter and subsequently diluted to 10 g/L.

Bulk Bottling: the retentate from the concentration and diafiltration step is filtered through a 0.22 μm pore size filter into 2 L single use teflon bottles. The bottles are frozen at −70° C.

Drug Product/Vial: the bottles of frozen Drug Substance are thawed at ambient temperature. After the Drug Substance is completely thawed, it is pooled in a sterile container, filtered, filled into vials, partially stoppered, and lyophilized. After completion of the freeze-drying process, the vials are stoppered and capped. The lyophilized Drug Product is stored at 2-8° C.

The CG53135-05 reference standard was prepared at Diosynth RTP Inc, using a 140L scale manufacturing process that was representative of the bulk drug substance manufacturing process (as described in the General Method of Manufacture). The reference standard was stored as 1 mL aliquots in 2 mL cryovials at −80° C.±15° C.

Purity of the final product was analyzed by SDS-PAGE, RP-HPLC, size exclusion-HPLC, and Western blot. Potency of the drug was measured by cell growth NIH 3T3 cells in response to CG53135-05. All data indicated that the final product is suitable for clinical uses.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

Thus, while the preferred embodiments of the invention have been illustrated and described, it is to be understood that this invention is capable of variation and modification, and should not be limited to the precise terms set forth. The inventors desire to avail themselves of such changes and alterations which may be made for adapting the invention to various usages and conditions. Such alterations and changes may include, for example, different pharmaceutical compositions for the administration of the proteins according to the present invention to a mammal; different amounts of protein in the compositions to be administered; different times and means of administering the proteins according to the present invention; and different materials contained in the administration dose including, for example, combinations of different proteins, or combinations of the proteins according to the present invention together with other biologically active compounds for the same, similar or differing purposes than the desired utility of those proteins specifically disclosed herein. Such changes and alterations also are intended to include modifications in the amino acid sequence of the specific desired proteins described herein in which such changes alter the sequence in a manner as not to change the desired potential of the protein, but as to change solubility of the protein in the pharmaceutical composition to be administered or in the body, absorption of the protein by the body, protection of the protein for either shelf life or within the body until such time as the biological action of the protein is able to bring about the desired effect, and such similar modifications. Accordingly, such changes and alterations are properly intended to be within the full range of equivalents, and therefore within the purview of the following claims.

The invention and the manner and process of making and using it have been thus described in such full, clear, concise and exact terms so as to enable any person skilled in the art to which it pertains, or with which it is most nearly connected, to make and use the same.

1. A method of preventing or treating inflammatory bowel disease comprising administering to a subject in need thereof an effective amount of a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

2. A method of preventing or treating inflammatory bowel disease comprising administering to a subject in need thereof an effective amount of a composition comprising a protein isolated from a cultured host cell containing an isolated nucleic acid molecule selected from the group consisting of:
(a) a nucleic acid molecule comprising a nucleotide sequence selected from the group consisting of SEQ ID NOs: 1, 3, 5, 6, 8, 9, 21, 23, 25, 27, 29, 31, 33, 35, 37, 39 and 41;
(b) a nucleic acid molecule encoding a protein comprising an amino acid sequence selected from the group consisting of SEQ ID NOs: 2, 4, 7, 10, 22, 24, 26, 28, 30, 32, 34, 36, 38, or 40; and
(c) a nucleic acid molecule hybridizes under stringent conditions to a nucleotide sequence of SEQ ID NO: 1, 3, 5, 6, 8, 9, 21, 23, 25, 27, 29, 31, 33, 35, 37, 39 or 41, or a complement of said nucleic acid molecule, and wherein said stringent conditions comprise a salt concentration from about 0.1 M to about 1.0 M sodium ion, a pH from about 7.0 to about 8.3, a temperature is at least about 60° C., and at least one wash in 0.2×SSC, 0.01% BSA.

3. The method of claim 2, wherein said host cell is a eukaryotic cell.

4. The method of claim 2, wherein said host cell is a prokaryotic cell.

5. The method of claim 4, wherein said prokaryotic cell is E. coli.

6. The method of claim 2, wherein said protein isolated from a cultured host cell has a purity of at least 98%.

7. The method of claim 1 or 2, wherein said composition further comprises a pharmaceutically acceptable carrier.

8. The method of claim 7, wherein said composition comprising 0.02-0.2 M acetate, 0.5-5% glycerol, 0.2-0.5 M arginine-HCl, and 0.005-5 mg/ml of said isolated protein.

9. The method of claim 8, wherein said composition comprising 0.04M acetate, 3% Glycerol (volume/volume), 0.2M Arginine-HCl at pH 5.3, and 0.8 mg/ml of said isolated protein.

10. The method of claim 7, wherein said composition comprising 0.01-1 M arginine in a salt form, sulfobutyl ether Beta-cyclodextrin sodium, or sucrose, about 0.01-0.1 M sodium phosphate monobasic (NaH₂PO₄.H₂O), about 0.01%-0.1% weight/volume (“w/v”) polysorbate 80 or polysorbate 20, and about 0.005 mg/ml to about 50 mg/ml of said isolated protein.

11. The method of claim 10, wherein said composition comprises an arginine in a salt form selected from the group consisting of arginine, arginine sulfate, arginine phosphate, and arginine hydrochloride.

12. The method of claim 10, said arginine in a salt form, sulfobutyl ether Beta-cyclodextrin sodium or sucrose is of 0.01-0.7 M.

13. The method of claim 10, wherein said composition comprises an arginine in a salt form at a concentration of 0.5 M.

14. The method of claim 10, wherein said sodium phosphate monobasic is 0.05 M.

15. The method of claim 10, wherein said polysorbate 80 or polysorbate 20 is 0.01% (w/v).

16. The method of claim 10, wherein said isolated protein is at a concentration of 5-30 mg/ml.

17. The method of claim 10, wherein said isolated protein is at a concentration of 10 mg/ml.

18. The method of claim 10, wherein said isolated protein comprises an amino acid sequence of SEQ ID NO:24.

19. The method of claim 10, wherein said isolated protein comprises an amino acid sequence of SEQ ID NO:2.

20. The method of claim 10, wherein said isolated protein comprises two or more proteins.

21. The method of claim 19, wherein said composition comprises a first protein comprising an amino acid sequence of SEQ ID NO:24, and a second protein comprising an amino acid sequence of SEQ ID NO:2.

22. The method of claim 20, wherein said composition further comprises an isolated protein comprising an amino acid sequence selected from the group consisting of SEQ ID NOs:26, 28, 30 and 32.

23. The method of claim 20, wherein said composition further comprises a third protein comprising an amino acid sequence of SEQ ID NO:28, a fourth protein comprising an amino acid sequence of SEQ ID NO:30, and a fifth protein comprising an amino acid sequence of SEQ ID NO:32.

24. The method of claim 10, wherein said composition is lyophilized or spray dried.

25. The method of claim 10, wherein said isolated protein is at least 98% of the total protein in the composition.

26. The method of claim 1 or 2, wherein said inflammatory bowel disease is Crohn's disease.

27. The method of claim 1 or 2, wherein said inflammatory bowel disease is ulcerative colitis.

28. The method of claim 1 or 2, wherein said subject is a mammal.

29. The method of claim 28, wherein said mammal is a human.

30. The method of claim 1 or 2, wherein said administering is a single dose administered at a dosage of 0.001-1 mg/kg, 0.01-0.5 mg/kg, 0.01-0.2 mg/kg, 0.03 mg/kg, 0.1 mg/kg, or 0.2 mg/kg.

31. The method of claim 1 or 2, wherein said administering is a multiple dosing administered with each unit dosage of 0.001-0.5 mg/kg, 0.01-0.2 mg/kg, 0.03 mg/kg, 0.1 mg/kg, or 0.2 mg/kg.

32. The method of claim 1 or 2, wherein said administering is parenteral administration.

33. The method of claim 32, wherein said parenteral administration is intravenous administration.

34. The method of claim 32, wherein said parenteral administration is subcutaneous administration.

35. The method of claim 1 or 2, wherein said administering is rectal administration.

36. The method of claim 1 or 2, wherein said administering is transdermal administration.

37. The method of claim 1 or 2, wherein said administering is transmucosal administration.

38. The method of claim 37, wherein said transmucosal administration is nasal administration.

39. A method of stimulating proliferation, differentiation or migration of epithelial cells or mesenchymal cells comprising administering to a subject in need thereof an effective amount of a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

40. The method of claim 39, wherein said composition further comprising a pharmaceutically acceptable carrier.

41. The method of claim 39, wherein said epithelial cells or mesenchymal cells locate at the alimentary tract of said subject.

42. The method claim 39, wherein said epithelial cells or mesenchymal cells locate at the pulmonary tract of said subject.

43. The method of claim 42, wherein said epithelial cells or mesenchymal cells locate at trachea.

44. The method of claim 39, wherein said subject is a mammal.

45. The method of claim 39, wherein said mammal is a human.

46. A method of preventing or treating irritable bowel syndrome comprising administering to a subject in need thereof an effective amount of a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.


SEQUENCE LISTING (57 sequences)

SEQ ID NO: 1 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 2 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 3 - DNA, 477 bp, Homo sapiens
  CDS (1)..(474)
atggctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 4 - protein, 158 aa, Homo sapiens
MAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 5 - DNA, 636 bp, Homo sapiens
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttga

SEQ ID NO: 6 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgcgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 7 - protein, 179 aa, Homo sapiens
MAPLAEVGGFLGGLEGLGQPGAAQLAHLHGILRRRQLYCRTGFHLQILPDGSAQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 8 - DNA, 636 bp, Homo sapiens
atggctccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 9 - DNA, 540 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 10 - protein, 179 aa, Homo sapiens
MAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 11 - DNA, 54 bp, Homo sapiens
  CDS (1)..(54)
atggctcccttagccgaagtcgggggctttctgggcggcctggagggcttgggc

SEQ ID NO: 12 - protein, 18 aa, Homo sapiens
MAPLAEVGGFLGGLEGLG

SEQ ID NO: 13 - DNA, 63 bp, Homo sapiens
  CDS (1)..(63)
gagcggccgccgctgctgggcgagcgcaggagcgcggcggagcggagcgcgcgcggcgggccg

SEQ ID NO: 14 - protein, 21 aa, Homo sapiens
ERPPLLGERRSAAERSARGGP

SEQ ID NO: 15 - DNA, 63 bp, Homo sapiens
  CDS (1)..(63)
cgcaggtattttgtggcacttaacaaagacggaactccaagagatggcgccaggtccaagagg

SEQ ID NO: 16 - protein, 21 aa, Homo sapiens
RRYFVALNKDGTPRDGARSKR

SEQ ID NO: 17 - DNA, 60 bp, Homo sapiens
  CDS (1)..(60)
cctagaccagtggatccagaaagagttccagaattgtacaaggacctactgatgtacact

SEQ ID NO: 18 - protein, 20 aa, Homo sapiens
PRPVDPERVPELYKDLLMYT

SEQ ID NO: 19 - DNA, 51 bp, Homo sapiens
  CDS (1)..(51)
atgaacgacaagggcgagctgtacggcagcgagaagctgaccagcgagtgc

SEQ ID NO: 20 - protein, 17 aa, Homo sapiens
MNDKGELYGSEKLTSEC

SEQ ID NO: 21 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacact

SEQ ID NO: 22 - protein, 211 aa, Homo sapiens [sequence omitted]

SEQ ID NO: 23 - DNA, 630 bp, Homo sapiens
  CDS (1)..(627)
ccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 24 - protein, 209 aa, Homo sapiens
PLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 25 - DNA, 612 bp, Homo sapiens
  CDS (1)..(609)
ggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 26 - protein, 203 aa, Homo sapiens
GFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 27 - DNA, 603 bp, Homo sapiens
  CDS (1)..(600)
ggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 28 - protein, 200 aa, Homo sapiens
GGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 29 - DNA, 594 bp, Homo sapiens
  CDS (1)..(591)
gagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 30 - protein, 197 aa, Homo sapiens
EGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 31 - DNA, 567 bp, Homo sapiens
  CDS (1)..(564)
cacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 32 - protein, 188 aa, Homo sapiens
HFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 33 - DNA, 402 bp, Homo sapiens
  CDS (1)..(402)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtcgac

SEQ ID NO: 34 - protein, 134 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVD

SEQ ID NO: 35 - DNA, 447 bp, Homo sapiens
  CDS (1)..(447)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 36 - protein, 149 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 37 - DNA, 396 bp, Homo sapiens
  CDS (1)..(396)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatgaagacactggccgcaggtattttgtggcacttaacaaagacggaactccaagagatggcgccaggtccaagaggcatcagaaatttacacatttcttacctagacca

SEQ ID NO: 38 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHEDTGRRYFVALNKDGTPRDGARSKRHQKFTHFLPRP

SEQ ID NO: 39 - DNA, 396 bp, Homo sapiens
  CDS (1)..(396)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagacca

SEQ ID NO: 40 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRP

SEQ ID NO: 41 - DNA, 537 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 42 - DNA, 24 bp, Artificial Sequence
  Description of Artifical Sequence Primer/Probe
agatcatctctgcctgagtatctt

SEQ ID NO: 43 - DNA, 27 bp, Artificial Sequence
  Description of Artifical Sequence Primer/Probe
ttcaaatgagattgtgggaaaattgct

SEQ ID NO: 44 - DNA, 17 bp, Artificial Sequence
  Description of Artifical Sequence Primer/Probe
gtgccagccaaggacag

SEQ ID NO: 45 - DNA, 21 bp, Artificial Sequence
  Description of Artifical Sequence Primer/Probe
cgttaagacatcagcctccag

SEQ ID NO: 46 - DNA, 20 bp, Artificial Sequence
  Description of Artifical Sequence Primer/Probe
tctctccgtaatggaagacc

SEQ ID NO: 47 - DNA, 20 bp, Artificial Sequence
  Description of Artifical Sequence Primer/Probe
gcattatgagacatccccac

SEQ ID NO: 48 - DNA, 18 bp, Artificial Sequence
  Description of Artifical Sequence Primer/Probe
ccaaccgcaagaagatga

SEQ ID NO: 49 - DNA, 21 bp, Artificial Sequence
  Description of Artifical Sequence Primer/Probe
gatcttcatgaggtagtcagt

SEQ ID NO: 50 - DNA, 22 bp, Artificial Sequence
  Description of Artifical Sequence Primer/Probe
gcaaatccttgctgttccaatc

SEQ ID NO: 51 - DNA, 22 bp, Artificial Sequence
  Description of Artifical Sequence Primer/Probe
ggagaaggcttcccagcttttg

SEQ ID NO: 52 - DNA, 23 bp, Artificial Sequence
  Description of Artifical Sequence Primer/Probe
gaagtttgcgtgctgccatggag

SEQ ID NO: 53 - DNA, 22 bp, Artificial Sequence
  Description of Artifical Sequence Primer/Probe
ccgcaattagaacagccttgtg

SEQ ID NO: 54 - DNA, 24 bp, Artificial Sequence
  Description of Artifical Sequence Primer/Probe
ctcttactgactggcatgaggatc

SEQ ID NO: 55 - DNA, 27 bp, Artificial Sequence
  Description of Artifical Sequence Primer/Probe
ctatgcagttgatgaagatgtcaaatt

SEQ ID NO: 56 - DNA, 25 bp, Artificial Sequence
  Description of Artifical Sequence Primer/Probe
cggtgctgagtatgtcgtggagtct

SEQ ID NO: 57 - DNA, 25 bp, Artificial Sequence
  Description of Artifical Sequence Primer/Probe
gttattatgggggtctgggatggaa